SEQUENCE LISTING (228 sequences)

SEQ ID NO: 1 - protein, 54 aa, Artificial Sequence
  Synthetic
  MISC_FEATURE (1)..(1): X is A,C,D,E,F,G,H,I,L,M,N,P,Q,R,S,T,V,W, or Y
  MISC_FEATURE (2)..(2): X is A,D,E,G,H,I,P, or T
  MISC_FEATURE (3)..(3): X is A,C,D,E,G,I,L,M,N,P,Q,S,T,V, or Y
  MISC_FEATURE (5)..(5): X is A,C,D,E,F,G,H,I,K,L,M,N,P,Q,R,S,T,V,W, or Y
  MISC_FEATURE (6)..(6): X is C,F,H,I,K,L,M,T,V,W, or Y
  MISC_FEATURE (7)..(7): X is A,C,I,L,T, or V
  MISC_FEATURE (8)..(8): X is A,C,D,E,F,G,H,I,K,L,M,N,Q,R,S,T,V, or Y
  MISC_FEATURE (9)..(9): X is A,D,E,F,G,H,I,K,L,M,S,W, or Y
  MISC_FEATURE (10)..(10): X is A,C,E,F,H,K,L,M,N,Q,R,S,W, or Y
  MISC_FEATURE (11)..(11): X is A,C,E,F,H,L,S,T,V, or Y
  MISC_FEATURE (12)..(12): X is A,C,D,E,F,G,H,I,K,L,N,Q,R,S, or T
  MISC_FEATURE (13)..(13): X is C,D,E,F,G,H,I,K,L,M,N,P,Q,R,S,T,V, OR W
  MISC_FEATURE (14)..(14): X is A,F,H,K,L,M,Q,W, or Y
  MISC_FEATURE (15)..(15): X isA,C,E,F,G,H,I,K,L,M,N,Q,R,S,T,V,W, or Y
  MISC_FEATURE (16)..(16): X is A,D,E,F,G,K,M,Q, or Y
  MISC_FEATURE (17)..(17): X is A,C,E,F,G,H,I,K,L,M,N,Q,R,S,T,V,W, or Y
  MISC_FEATURE (18)..(18): X is A,C,F,G,I,K,L,M,N,P,Q,R,T,V,W, or Y
  MISC_FEATURE (19)..(19): X is A,C,E,H,I,K,L,M,N,Q,R,S,T, or V
  MISC_FEATURE (20)..(20): X is A,D,E,I,K,L,N,P,Q,R,S,T,V, or Y
  MISC_FEATURE (21)..(21): X is H,Q, or V
  MISC_FEATURE (22)..(22): X is A,E,I,K,M,N,P,Q,R,S,T, or V
  MISC_FEATURE (23)..(23): X is A,D,E,G,H,I,K,L,N,Q,R,S,T,V, or Y
  MISC_FEATURE (24)..(24): X is A,C,G, or R
  MISC_FEATURE (25)..(25): X is I,L,M, or V
  MISC_FEATURE (26)..(26): X is A,E,G,H,I,K,M,S, or V
  MISC_FEATURE (27)..(27): X is A,C,D,E,F,H,I,K,L,M,N,Q,R,T,V,W, or Y
  MISC_FEATURE (28)..(28): X is A,C,I,L,N, or V
  MISC_FEATURE (29)..(29): X is A,Q,R,W, or Y
  MISC_FEATURE (30)..(30): X is H,I,K,L,M,N,P, or V
  MISC_FEATURE (31)..(31): X is C,F,H,I,L,M,V,W, or Y
  MISC_FEATURE (32)..(32): X is A or P
  MISC_FEATURE (33)..(33): X is F,H,K,L,M,N,R,V, or W
  MISC_FEATURE (34)..(34): X is A,G,H,K,P,Q,R,S,T, or Y
  MISC_FEATURE (35)..(35): X is A,C,D,E,F,G,H,I,K,L,M,N,Q,R,S,T,V,W, or Y
  MISC_FEATURE (36)..(36): X is D,G,H,K,N, or Q
  MISC_FEATURE (37)..(37): X is A,E,F,G,H,I,K,L,M,Q,R,S,T,V,W, or Y
  MISC_FEATURE (38)..(38): X is E,P, or Q
  MISC_FEATURE (39)..(39): X is A,G,I,L,M,T, or V
  MISC_FEATURE (41)..(41): X is F,I,K,M,P,Q,R,W, or Y
  MISC_FEATURE (42)..(42): X is F,L, or Y
  MISC_FEATURE (43)..(43): X is F,G,I,L,M,T, or V
  MISC_FEATURE (44)..(44): X is F,W, or Y
  MISC_FEATURE (45)..(45): X is A,C,D,E,F,G,H,I,L,M,Q,S,T,V,W, or Y
  MISC_FEATURE (46)..(46): X is F or Y
  MISC_FEATURE (48)..(48): X is E,G,N,Q,S, or T
  MISC_FEATURE (49)..(49): X is C,D,E,F,H,I,L,N,R,T, or Y
  MISC_FEATURE (50)..(50): X is I,K,N,R, or V
  MISC_FEATURE (51)..(51): X is D,E,K,N,P, or R
  MISC_FEATURE (52)..(52): X is A,D,E,G,H,K,L,N,P,Q,R,S,T, or Y
  MISC_FEATURE (53)..(53): X is A,D,E,G,H,K,M,N,P,Q,R,S,T,V, or Y
  MISC_FEATURE (54)..(54): X is A,D,E,G,H,I,K,L,M,N,P,Q,R,S,T,V, or W
XXXEXXXXXXXXXXXXXXXXXXXXXXXXXXXXXXXXXXXWXXXXXXMXXXXXXX

SEQ ID NO: 2 - protein, 54 aa, Artificial Sequence
  Synthetic
  MISC_FEATURE (1)..(1): X is A,C,D,E,G,H,I,K,L,M,N,P,Q,S,T, or V
  MISC_FEATURE (2)..(2): X is C or E
  MISC_FEATURE (3)..(3): X is A,C,D,E,G,K,N,P,S,V, or Y
  MISC_FEATURE (4)..(4): X is C or E
  MISC_FEATURE (5)..(5): X is A,C,D,E,G,I,K,L,M,N,P, or Q
  MISC_FEATURE (6)..(6): X is C,F,I,L,M,V,W, or Y
  MISC_FEATURE (7)..(7): X is L or V
  MISC_FEATURE (8)..(8): X is A,C,D,E,F,G,H,I,K,L,M,N,Q,R,S,T,V,W, or Y
  MISC_FEATURE (9)..(9): X is C,D,E,F,I,L,M, or V
  MISC_FEATURE (10)..(10): X is L,M, or Q
  MISC_FEATURE (11)..(11): X is C,I,K, or V
  MISC_FEATURE (12)..(12): X is C,D,H, or K
  MISC_FEATURE (13)..(13): X is A,D,E,F,G,H,I,K,L,M,N,Q,R,T,V, or Y
  MISC_FEATURE (14)..(14): X is V or Y
  MISC_FEATURE (15)..(15): X is A,C,D,E,F,G,H,M,N,Q,S,T,V,W, or Y
  MISC_FEATURE (16)..(16): X is A,C,D,E,G,H,L,M,N,Q, or S
  MISC_FEATURE (17)..(17): X is C,G,H,K,Q, or R
  MISC_FEATURE (18)..(18): X is A,C,F,K,N,R,S,T, or V
  MISC_FEATURE (19)..(19): X is C,F,I,L,M,T, or V
  MISC_FEATURE (20)..(20): X is A,C,D,L,M,N,Q,S, or T
  MISC_FEATURE (21)..(21): X is H,I,K,Q,V, or Y
  MISC_FEATURE (22)..(22): X is C,E,F,H,I,K,L,P,Q,S,T, or V
  MISC_FEATURE (23)..(23): X is C,D,E,F,G,H,K,M,N,Q, or R
  MISC_FEATURE (24)..(24): X is A,D, or V
  MISC_FEATURE (25)..(25): X is M or V
  MISC_FEATURE (26)..(26): X is E,F,G,H,L,M,R,S,V, or Y
  MISC_FEATURE (27)..(27): X is C,D,E,H,M,N,R, or T
  MISC_FEATURE (28)..(28): X is C, I or V
  MISC_FEATURE (29)..(29): X is G,H,Q,R, or W
  MISC_FEATURE (30)..(30): X is C,D,E,F,I,K,L,M,N,Q,T,V, or W
  MISC_FEATURE (31)..(31): X is C,F,I,L,M,V,W, or Y
  MISC_FEATURE (32)..(32): X is A,D,H, or S
  MISC_FEATURE (33)..(33): X is A,F,G,H,I,K,L,N,Q,R,T,V, or Y
  MISC_FEATURE (34)..(34): X is A,C,D,E,F,G,H,I,K,L,M,N,Q,S,T,V,W, or Y
  MISC_FEATURE (35)..(35): X is A,C,E,F,G,H,I,K,L,M,N,Q,S,T,V,W, or Y
  MISC_FEATURE (36)..(36): X is C,G,H,N,P, or Q
  MISC_FEATURE (37)..(37): X is C,E,G,I,L,Q,T,V, or W
  MISC_FEATURE (39)..(39): X is A,G,I,M,T, or V
  MISC_FEATURE (41)..(41): X is I,K,L,M,Q, or W
  MISC_FEATURE (42)..(42): X is L or M
  MISC_FEATURE (43)..(43): X is I or V
  MISC_FEATURE (44)..(44): X is Q or W
  MISC_FEATURE (45)..(45): X is C,E,F,H,L,M,Q,V,W, or Y
  MISC_FEATURE (48)..(48): X is A,F,I,K,L,M,Q,V, or Y
  MISC_FEATURE (50)..(50): X is T or V
  MISC_FEATURE (51)..(51): X is A,D,K,N,Q, or T
  MISC_FEATURE (52)..(52): X is A,G,N,R, or S
  MISC_FEATURE (53)..(53): X is C,D,I,M,Q, or Y
  MISC_FEATURE (54)..(54): X is A,E,I,K,N,P,Q, or V
XXXXXXXXXXXXXXXXXXXXXXXXXXXXXXXXXXXXXEXWXXXXXFMXCXXXXX

SEQ ID NO: 3 - protein, 54 aa, Artificial Sequence
  Synthetic
  MISC_FEATURE (1)..(1): X is A,C,D,E,G,H,I,K,L,M,N,P,Q,S,T, or V
  MISC_FEATURE (2)..(2): X is A,C,D,E,G,H,I,P, or T
  MISC_FEATURE (3)..(3): X is A,C,D,E,G,I,L,N,P,S,V,  or  Y
  MISC_FEATURE (4)..(4): X is C or E
  MISC_FEATURE (5)..(5): X is A,C,D,E,F,G,H,I,K,L,M,N,P,Q,R,S,T,V,W, or Y
  MISC_FEATURE (6)..(6): X is C,F,I,L,M,V,W, or Y
  MISC_FEATURE (7)..(7): X is A,C,I,L,T, or V
  MISC_FEATURE (8)..(8): X is A,C,D,E,F,G,H,I,K,L,M,N,Q,R,S,T,V,W, or Y
  MISC_FEATURE (9)..(9): X is C,E,G,H,I,L,M,S, or Y
  MISC_FEATURE (10)..(10): X is A,C,E,F,H,K,L,M,N,Q,R,S,W, or Y
  MISC_FEATURE (11)..(11): X is C,E,F,H,L,V, or Y
  MISC_FEATURE (12)..(12): X is A,C,F,G,H,K,L,Q,R,S, or T
  MISC_FEATURE (13)..(13): X is A,C,D,F,H,I,K,L,M,N,Q,R,S,T,V, or W
  MISC_FEATURE (14)..(14): X is K or Y
  MISC_FEATURE (15)..(15): X is A,C,E,H,N,S,T,V,W, or Y
  MISC_FEATURE (16)..(16): X is A,C,D,E,G,M,N,Q, or S
  MISC_FEATURE (17)..(17): X is A,C,E,F,G,H,I,K,L,M,N,Q,R,S,T,V,W, or Y
  MISC_FEATURE (18)..(18): X is C,F,I,K,L,M,N,P,R,S,T,V, or W
  MISC_FEATURE (19)..(19): X is A,C,F,H,I,K,L,M,N,Q,R,S,T, or V
  MISC_FEATURE (20)..(20): X is A,C,D,E,I,K,L,M,Q,R,S,T, or V
  MISC_FEATURE (21)..(21): X is H,I,Q, or V
  MISC_FEATURE (22)..(22): X is A,C,E,I,K,N,P,Q,S, or T
  MISC_FEATURE (23)..(23): X is A,C,D,E,F,G,H,I,K,L,N,Q,R,S,T,V, or Y
  MISC_FEATURE (24)..(24): X is A,C,G, or R
  MISC_FEATURE (26)..(26): X is E,F,G,L,M,S,V, or Y
  MISC_FEATURE (27)..(27): X is A,C,D,E,F,H,I,K,L,M,N,Q,R,T,V,W, or Y
  MISC_FEATURE (28)..(28): X is A,C,I,N, or V
  MISC_FEATURE (29)..(29): X is Q,R,W, or Y
  MISC_FEATURE (30)..(30): X is C,E,H,I,K,L,M,Q, or V
  MISC_FEATURE (31)..(31): X is C,F,I,L,M,V,W, or Y
  MISC_FEATURE (32)..(32): X is A or S
  MISC_FEATURE (33)..(33): X is F,H,I,K,L,M,N,Q,R,T,V,W, or Y
  MISC_FEATURE (34)..(34): X is A,C,D,E,F,G,H,I,K,L,M,Q,R,S,T,V,W, or Y
  MISC_FEATURE (35)..(35): X is A,C,E,F,G,H,I,K,L,M,N,Q,R,S,T,V,W, or Y
  MISC_FEATURE (36)..(36): X is G or H
  MISC_FEATURE (37)..(37): X is A,C,E,F,H,L,T, or Y
  MISC_FEATURE (38)..(38): X is E or Q
  MISC_FEATURE (39)..(39): X is A or G
  MISC_FEATURE (41)..(41): X is F,I,K,L,M,Q,R,W, or Y
  MISC_FEATURE (42)..(42): X is L or M
  MISC_FEATURE (43)..(43): X is I, T or V
  MISC_FEATURE (43)..(43): X is I, T or V
  MISC_FEATURE (45)..(45): X is A,C,E,F,G,H,I,L,M,Q,S,T,V,W, or Y
  MISC_FEATURE (46)..(46): X is F or Y
  MISC_FEATURE (48)..(48): X is E,F,G,I,K,L,M,N,Q,S,T, or Y
  MISC_FEATURE (49)..(49): X is C,D,H,L, or R
  MISC_FEATURE (50)..(50): X is I,K,N,R, or V
  MISC_FEATURE (51)..(51): X is A,K,N,Q,R, or T
  MISC_FEATURE (52)..(52): X is A,D,E,G,H,K,L,N,P,Q,R,S, or T
  MISC_FEATURE (53)..(53): X is A,C,D,E,G,H,K,M,N,P,Q,R,S,V, or Y
  MISC_FEATURE (54)..(54): X is A,D,E,G,H,I,K,L,M,N,P,Q,R,S,T,V, or W
XXXXXXXXXXXXXXXXXXXXXXXXVXXXXXXXXXXXXXXWXXXWXXMXXXXXXX

SEQ ID NO: 4 - protein, 54 aa, Artificial Sequence
  Synthetic
  MISC_FEATURE (1)..(1): X is A,C,D,E,G,H,I,K,M,N,P,Q,S,T, or V
  MISC_FEATURE (2)..(2): X is C or E
  MISC_FEATURE (3)..(3): X is A,C,D,E,G,P, or V
  MISC_FEATURE (4)..(4): X is C or E
  MISC_FEATURE (5)..(5): X is A,C,D,E,G,I,K,M,N,P, or Q
  MISC_FEATURE (6)..(6): X is C,F,I,L,M,V,W, or Y
  MISC_FEATURE (7)..(7): X is L or V
  MISC_FEATURE (8)..(8): X is A,C,D,E,F,G,H,I,K,L,M,N,Q,R,S,T,V,W, or Y
  MISC_FEATURE (9)..(9): X is C,E,L, or M
  MISC_FEATURE (10)..(10): X is L,M, or Q
  MISC_FEATURE (12)..(12): X is C or K
  MISC_FEATURE (13)..(13): X is A,H,I,K,M,N,Q,R,T, or V
  MISC_FEATURE (15)..(15): X is A,C,E,H,N,S,T,V,W, or Y
  MISC_FEATURE (16)..(16): X is A,C,D,E,G,M,N,Q, or S
  MISC_FEATURE (17)..(17): X is G, H or K
  MISC_FEATURE (18)..(18): X is A,C,K,N,R,S, or T
  MISC_FEATURE (19)..(19): X is C,F,I,L,T, or V
  MISC_FEATURE (20)..(20): X is A,C,L,M,Q,S, or T
  MISC_FEATURE (21)..(21): X is H, I or Q
  MISC_FEATURE (22)..(22): X is C,E,I,K,P,Q,S, or T
  MISC_FEATURE (23)..(23): X is C,D,E,F,G,H,K,N,Q, or R
  MISC_FEATURE (26)..(26): X is E,F,G,L,M,S,V, or Y
  MISC_FEATURE (27)..(27): X is C,D,E,H,M,N, or R
  MISC_FEATURE (28)..(28): X is C or I
  MISC_FEATURE (29)..(29): X is Q, R or W
  MISC_FEATURE (30)..(30): X is C,E,K,L,M,Q, or V
  MISC_FEATURE (31)..(31): X is C,F,I,L,M,V,W, or Y
  MISC_FEATURE (32)..(32): X is A or S
  MISC_FEATURE (33)..(33): X is F,H,I,L,N,Q,R,T, or Y
  MISC_FEATURE (34)..(34): X is A,C,D,E,F,G,H,I,K,L,M,Q,T,V,W, or Y
  MISC_FEATURE (35)..(35): X is A,C,E,F,G,H,I,K,L,M,N,Q,S,T,V,W or Y
  MISC_FEATURE (36)..(36): X is G or H
  MISC_FEATURE (37)..(37): X is C or L
  MISC_FEATURE (41)..(41): X is I,K,L,M,Q, or W
  MISC_FEATURE (42)..(42): X is L or M
  MISC_FEATURE (43)..(43): X is I or V
  MISC_FEATURE (45)..(45): X is C,E,F,H,L,M,Q,V,W, or Y
  MISC_FEATURE (48)..(48): X is F,I,K,L,M,Q, or Y
  MISC_FEATURE (51)..(51): X is A,K,N,Q, or T
  MISC_FEATURE (52)..(52): X is A,G,N,R, or S
  MISC_FEATURE (53)..(53): X is C or D
  MISC_FEATURE (54)..(54): X is I,N,P, or V
XXXXXXXXXXCXXYXXXXXXXXXAVXXXXXXXXXXXXEAWXXXWXFMXCVXXXX

SEQ ID NO: 5 - protein, 54 aa, Artificial Sequence
  Synthetic
  MISC_FEATURE (1)..(1): X is D,E,F,H,I,M,N,T, or Y
  MISC_FEATURE (2)..(2): X is D or E
  MISC_FEATURE (5)..(5): X is K or Y
  MISC_FEATURE (8)..(8): X is E,F,K,L,N, or R
  MISC_FEATURE (9)..(9): X is A,D,E,F,I, or K
  MISC_FEATURE (11)..(11): X is A,C,S,T,V, or Y
  MISC_FEATURE (12)..(12): X is D,E,I,K, or R
  MISC_FEATURE (13)..(13): X is E,H,K,N, or T
  MISC_FEATURE (14)..(14): X is F,K,M,W, or Y
  MISC_FEATURE (15)..(15): X is A,E,F,G,H,L,N,Q, or Y
  MISC_FEATURE (16)..(16): X is E,F,G,K,M, or Y
  MISC_FEATURE (17)..(17): X is A,G, or N
  MISC_FEATURE (18)..(18): X is G,K,L,Q,R, or T
  MISC_FEATURE (19)..(19): X is L or N
  MISC_FEATURE (20)..(20): X is A,E,Q,S, or Y
  MISC_FEATURE (21)..(21): X is H or Q
  MISC_FEATURE (22)..(22): X is K,P,Q, or R
  MISC_FEATURE (23)..(23): X is E or H
  MISC_FEATURE (27)..(27): X is E, I or N
  MISC_FEATURE (29)..(29): X is Q or R
  MISC_FEATURE (33)..(33): X is K or R
  MISC_FEATURE (34)..(34): X is K,P,S, or T
  MISC_FEATURE (35)..(35): X is A,C,D,G,H,K,M,N,Q,R, or Y
  MISC_FEATURE (36)..(36): X is G,H, or N
  MISC_FEATURE (37)..(37): X is I or L
  MISC_FEATURE (45)..(45): X is F, V, W or Y
  MISC_FEATURE (48)..(48): X is E or Q
  MISC_FEATURE (49)..(49): X is C,D,F,H,I,L,N,T, or Y
  MISC_FEATURE (51)..(51): X is E or K
  MISC_FEATURE (52)..(52): X is N, P or R
XXPEXYLXXLXXXXXXXXXXXXXAVEXIXKYAXXXXXEAWKLVWXFMXXVXXDI

SEQ ID NO: 6 - protein, 54 aa, Artificial Sequence
  Synthetic
  MISC_FEATURE (1)..(1): X is C,D,E,G,H,I,K,M,N,P,Q,S,T, or V
  MISC_FEATURE (2)..(2): X is C or E
  MISC_FEATURE (3)..(3): X is A,E,P, or V
  MISC_FEATURE (4)..(4): X is C or E
  MISC_FEATURE (5)..(5): X is A,C,D,I,K,M,N, or Q
  MISC_FEATURE (6)..(6): X is F or Y
  MISC_FEATURE (8)..(8): X is A,C,D,E,G,I,K,L,M,N,Q,R,S,T,V, or W
  MISC_FEATURE (9)..(9): X is C,E,L, or M
  MISC_FEATURE (10)..(10): X is L,M, or Q
  MISC_FEATURE (12)..(12): X is C or K
  MISC_FEATURE (13)..(13): X is A,H,I,K,M,N,Q,R,T, or V
  MISC_FEATURE (15)..(15): X is A,C,E,H,S,T,W, or Y
  MISC_FEATURE (16)..(16): X is A,C,D,E,G,M,N,Q, or S
  MISC_FEATURE (17)..(17): X is G,H, or K
  MISC_FEATURE (18)..(18): X is A,C,K,N,R,S, or T
  MISC_FEATURE (19)..(19): X is C,F,I,L,T, or V
  MISC_FEATURE (20)..(20): X is A,C,L,M,Q,S, or T
  MISC_FEATURE (21)..(21): X is H,I, or Q
  MISC_FEATURE (22)..(22): X is C,E,I,K,P,Q,S, or T
  MISC_FEATURE (23)..(23): X is C,D,E,F,G,H,K,N,Q, or R
  MISC_FEATURE (26)..(26): X is E,F,G,L,M,S,V, or Y
  MISC_FEATURE (27)..(27): X is C,D,E,H,M,N, or R
  MISC_FEATURE (29)..(29): X is Q or R
  MISC_FEATURE (30)..(30): X is C,E,K,L,M,Q, or V
  MISC_FEATURE (32)..(32): X is A or S
  MISC_FEATURE (33)..(33): X is F,H,I,L,N,Q,R,T, or Y
  MISC_FEATURE (34)..(34): X is A,C,D,F,G,H,I,K,L,M,Q,T,V,W, or Y
  MISC_FEATURE (35)..(35): X is A,E,K,M,N,Q,T, or W
  MISC_FEATURE (36)..(36): X is G or H
  MISC_FEATURE (37)..(37): X is C or L
  MISC_FEATURE (41)..(41): X is K,M, or Q
  MISC_FEATURE (42)..(42): X is L or M
  MISC_FEATURE (43)..(43): X is I or V
  MISC_FEATURE (45)..(45): X is C,F,V, or Y
  MISC_FEATURE (48)..(48): X is F,I,K,L,M,Q, or Y
  MISC_FEATURE (51)..(51): X is A,K,N,Q, or T
  MISC_FEATURE (52)..(52): X is A,N,R, or S
  MISC_FEATURE (53)..(53): X is C or D
  MISC_FEATURE (54)..(54): X is I,N,P, or V
XXXXXXLXXXCXXYXXXXXXXXXAVXXIXXYXXXXXXEAWXXXWXFMXCVXXXX

SEQ ID NO: 7-9 - skipped (3 entries)

SEQ ID NO: 10 - protein, 54 aa, Artificial Sequence
  Synthetic
MESEKYLRELVKKYYEGKLSVQEAVEEVRKYARKKGLEAWMLTWMFMELVKRYI

SEQ ID NO: 11 - protein, 54 aa, Artificial Sequence
  Synthetic
MEPEKYLREKVKKYYEGKLSQPEAVEEIRKYARKKGLEAWKLVWYFMELVKRDI

SEQ ID NO: 12 - protein, 54 aa, Artificial Sequence
  Synthetic
MEEEKYLRELVKKYYEQKLSHQEAVEIIRKYARKKGLEAWKLVWAFMQLVKRDI

SEQ ID NO: 13 - protein, 54 aa, Artificial Sequence
  Synthetic
MEEEKYVRELVKKYYEGKLSQPEAVEEIRKYARKKGLEAWKLIWAFMQLVKRDI

SEQ ID NO: 14 - protein, 54 aa, Artificial Sequence
  Synthetic
MEEEKYVRELVKKYYEGKLSHPEAVEEIRKYARKKGLEAWKLVWAFMQLVKRDI

SEQ ID NO: 15 - protein, 54 aa, Artificial Sequence
  Synthetic
MEEEKYVREQVKKYYEKKLSQPEAVEIIRKYARKKGLEAWKLIWAFMQLVKRDI

SEQ ID NO: 16 - protein, 54 aa, Artificial Sequence
  Synthetic
MEPEKYVRELVKKYYEKKLSQPEAVEEIRKYARKKGLEAWMLVWHFMQLVKRDI

SEQ ID NO: 17 - protein, 54 aa, Artificial Sequence
  Synthetic
MEEEKYLRELVKKYYEKKLSQPEAVEIIRKYARKKGLEAWKLVWAFMQLVKRDI

SEQ ID NO: 18 - protein, 54 aa, Artificial Sequence
  Synthetic
MEEEKYVRELVKKYYEKKLSQPEAVEEIRKYARKKGLEAWKLVWAFMELVKRNI

SEQ ID NO: 19 - protein, 54 aa, Artificial Sequence
  Synthetic
MEEEKYLRELVKKYYEQKLSQPEAVEIIRKYARKKGEEAWYLIWMFMELVKRDI

SEQ ID NO: 20 - protein, 54 aa, Artificial Sequence
  Synthetic
MEEEKYLREQVKKYYEGKLSVVEAVEEVRKYARKKGLEAWKLIWAFMQLVKRDI

SEQ ID NO: 21 - protein, 54 aa, Artificial Sequence
  Synthetic
MEEEKYVRELVKKYYEGKLSHQEAVVEIRKYARKKGLEAWKLVWMFMQLVKRNI

SEQ ID NO: 22 - protein, 54 aa, Artificial Sequence
  Synthetic
MEPEKYVRELVKKYYEQKLSQQEAVEIIRKYARKKGLEAWMLVWAFMQLVKRDI

SEQ ID NO: 23 - protein, 54 aa, Artificial Sequence
  Synthetic
MEPEKYVREKVKKYYEGKLSQPEAVEEIRKYARKKGLEAWKLIWHFMQLVKRDI

SEQ ID NO: 24 - protein, 54 aa, Artificial Sequence
  Synthetic
MEEEKYLRELVKKYYEQKLSQPEAVEIVRKYARKKGLEAWKLIWAFMELVKRYI

SEQ ID NO: 25 - protein, 54 aa, Artificial Sequence
  Synthetic
MEEEKYVRELCKKYYEGKLSQPEAVEEIRKYARKKGLEAWKLIWAFMQCVKRDI

SEQ ID NO: 26 - protein, 54 aa, Artificial Sequence
  Synthetic
MEEEKCVRELCKKYYEGKLSQPEAVEEIRKCARKKGLEAWKLIWAFMQCVKRDI

SEQ ID NO: 27 - protein, 54 aa, Artificial Sequence
  Synthetic
MEEEKCVRELCKKYYEGKLSHPEAVEEIRKCARKKGLEAWKLVWAFMQCVKRDI

SEQ ID NO: 28 - protein, 54 aa, Artificial Sequence
  Synthetic
MEEEKCVREQCKKYYEKKLSQPEAVEIIRKCARKKGLEAWKLIWAFMQCVKRDI

SEQ ID NO: 29 - protein, 54 aa, Artificial Sequence
  Synthetic
MEPEKCVRELCKKYYEKKLSQPEAVEEIRKCARKKGLEAWMLVWHFMQCVKRDI

SEQ ID NO: 30 - protein, 54 aa, Artificial Sequence
  Synthetic
MEEEKYVRELCKKYYEKKLSQPEAVEEIRKYARKKGLEAWKLVWAFMECVKRNI

SEQ ID NO: 31 - protein, 54 aa, Artificial Sequence
  Synthetic
MEEEKYVRELCKKYYEGKLSHQEAVVEIRKYARKKGLEAWKLVWMFMQCVKRNI

SEQ ID NO: 32 - protein, 54 aa, Artificial Sequence
  Synthetic
MEEEKCVRELCKKYYEGKLSHQEAVVEIRKCARKKGLEAWKLVWMFMQCVKRNI

SEQ ID NO: 33 - protein, 54 aa, Artificial Sequence
  Synthetic
MEPEKYVREQVKKYYEQKLSHQEAVEEVRKYARKKGLEAWKLTWYFMQLVKREI

SEQ ID NO: 34 - protein, 54 aa, Artificial Sequence
  Synthetic
MEEEKYVRELVKKYYEQKLSHPEAVEEIRKYARKKGLEAWKLVWYFMQLVKRNI

SEQ ID NO: 35 - protein, 54 aa, Artificial Sequence
  Synthetic
MEEEKYVRELVKKYYEGKLSHPEAVEEIRKYARKKGEEAWKLVWYFMQLVKRDI

SEQ ID NO: 36 - protein, 54 aa, Artificial Sequence
  Synthetic
MEPEKYVRELVKKYYEKKLSQPEAVEEMRKYARKKGLEAWKLVWHFMQLVKREI

SEQ ID NO: 37 - protein, 54 aa, Artificial Sequence
  Synthetic
MEPEKYLRELVKKYYEKKLSQQEAVEEIRKYARKKGLEAWKLVWYFMQLVKREI

SEQ ID NO: 38 - protein, 54 aa, Artificial Sequence
  Synthetic
MEPEKYLRELVKKYYEKKLSQPEAVEIIRKYARKKGLEAWKLVWYFMELVKREI

SEQ ID NO: 39 - protein, 54 aa, Artificial Sequence
  Synthetic
MEPEKYLRELVKKYYEQKLSQQEAVEEIRKYARKKGLEAWKLIWYFMELVKRDI

SEQ ID NO: 40 - protein, 54 aa, Artificial Sequence
  Synthetic
MEPEKYLREMVKKYYEKKLSQQEAVEEIRKYARKKGLEAWKLVWYFMELVKRYI

SEQ ID NO: 41 - protein, 54 aa, Artificial Sequence
  Synthetic
MEEEKYVRELVKKYYEQKLSQQEAVEEIRKYARKKGLEAWKLVWYFMELVKRNI

SEQ ID NO: 42 - protein, 54 aa, Artificial Sequence
  Synthetic
MEPEKYLRELVKKYYEQKLSQQEAVEIIRKYARKKGLEAWKLIWYFMQLVKRNI

SEQ ID NO: 43 - protein, 54 aa, Artificial Sequence
  Synthetic
MEPEKYVRELVKKYYEGKLSQPEAVEEIRKYARKKGLEAWKLVWYFMQLVKRNI

SEQ ID NO: 44 - protein, 54 aa, Artificial Sequence
  Synthetic
MEPEKYLRELVKKYYEGKLSQQEAVEEIRKYARKKGLEAWKLIWYFMELVKREI

SEQ ID NO: 45 - protein, 54 aa, Artificial Sequence
  Synthetic
MEEEKYVREQVKKYYEGKLSQQEAVEIIRKYARKKGLEAWKLIWYFMQLVKRDI

SEQ ID NO: 46 - protein, 54 aa, Artificial Sequence
  Synthetic
MEPEKYLRELVKKYYEGKLSQQEAVEEIRKYARKKGLEAWKLVWYFMQLVKRDI

SEQ ID NO: 47 - protein, 54 aa, Artificial Sequence
  Synthetic
MEPEKYVREQCKKYYEQKLSHQEAVEEVRKYARKKGLEAWKLTWYFMQCVKREI

SEQ ID NO: 48 - protein, 54 aa, Artificial Sequence
  Synthetic
MEEEKCVRELVKKYYEGKLSHPEAVEEIRKCARKKGEEAWKLVWYFMQLVKRDI

SEQ ID NO: 49 - protein, 54 aa, Artificial Sequence
  Synthetic
MEEEKYVRELCKKYYEGKLSHPEAVEEIRKYARKKGEEAWKLVWYFMQCVKRDI

SEQ ID NO: 50 - protein, 54 aa, Artificial Sequence
  Synthetic
MEEEKCVRELCKKYYEGKLSHPEAVEEIRKCARKKGEEAWKLVWYFMQCVKRDI

SEQ ID NO: 51 - protein, 54 aa, Artificial Sequence
  Synthetic
MEPEKCVRELVKKYYEKKLSQPEAVEEMRKCARKKGLEAWKLVWHFMQLVKREI

SEQ ID NO: 52 - protein, 54 aa, Artificial Sequence
  Synthetic
MEPEKYVRELCKKYYEKKLSQPEAVEEMRKYARKKGLEAWKLVWHFMQCVKREI

SEQ ID NO: 53 - protein, 54 aa, Artificial Sequence
  Synthetic
MEPEKYVRECCKKYYEKKLSQPECVEEMRKYARKKGLEAWKLVWHFMQCVKREI

SEQ ID NO: 54 - protein, 54 aa, Artificial Sequence
  Synthetic
MEPEKCVRELCKKYYEKKLSQPEAVEEMRKCARKKGLEAWKLVWHFMQCVKREI

SEQ ID NO: 55 - protein, 54 aa, Artificial Sequence
  Synthetic
MEPEKCLRELVKKYYEQKLSQQEAVEEIRKCARKKGLEAWKLIWYFMELVKRDI

SEQ ID NO: 56 - protein, 54 aa, Artificial Sequence
  Synthetic
MEPEKYLRELCKKYYEQKLSQQEAVEEIRKYARKKGLEAWKLIWYFMECVKRDI

SEQ ID NO: 57 - protein, 54 aa, Artificial Sequence
  Synthetic
MEPEKCLREMVKKYYEKKLSQQEAVEEIRKCARKKGLEAWKLVWYFMELVKRYI

SEQ ID NO: 58 - protein, 54 aa, Artificial Sequence
  Synthetic
MEPEKYLRELCKKYYEQKLSQQEAVEIIRKYARKKGLEAWKLIWYFMQCVKRNI

SEQ ID NO: 59 - protein, 54 aa, Artificial Sequence
  Synthetic
MEPEKCVRELVKKYYEGKLSQPEAVEEIRKCARKKGLEAWKLVWYFMQLVKRNI

SEQ ID NO: 60 - protein, 54 aa, Artificial Sequence
  Synthetic
MEPEKCLRELVKKYYEGKLSQQEAVEEIRKCARKKGLEAWKLVWYFMQLVKRDI

SEQ ID NO: 61 - protein, 54 aa, Artificial Sequence
  Synthetic
MEPEKYLRELCKKYYEGKLSQQEAVEEIRKYARKKGLEAWKLVWYFMQCVKRDI

SEQ ID NO: 62 - protein, 54 aa, Artificial Sequence
  Synthetic
MEPEKCLRELCKKYYEGKLSQQEAVEEIRKCARKKGLEAWKLVWYFMQCVKRDI

SEQ ID NO: 63 - protein, 54 aa, Artificial Sequence
  Synthetic
PEPEKYLRELCKKYYEGKLSQQEAVEEIRKYARKKGLEAWKLVWYFMQCVKRDI

SEQ ID NO: 64 - protein, 54 aa, Artificial Sequence
  Synthetic
TEPEKYLRELCKKYYEGKLSQQEAVEEIRKYARKKGLEAWKLVWYFMQCVKRDI

SEQ ID NO: 65 - protein, 54 aa, Artificial Sequence
  Synthetic
MEPEKYLQELCKKYYEGKLSQQEAVEEIRKYARKKGLEAWKLVWYFMQCVKRDI

SEQ ID NO: 66 - protein, 54 aa, Artificial Sequence
  Synthetic
MEPEKYLRELVKKYYEGKLSQQEAVEEIRKYARKKGLEAWKLVWYFMQCVKRDI

SEQ ID NO: 67 - protein, 54 aa, Artificial Sequence
  Synthetic
MEPEKYLRELCKKYYEGKLSQQEAVEEIRKYARKWGLEAWKLVWYFMQCVKRDI

SEQ ID NO: 68 - protein, 54 aa, Artificial Sequence
  Synthetic
MEPEKYLRELCKKYYEGKLSQQEAVEEIRKYARKKGLEAWKLVWCFMQAVKRDI

SEQ ID NO: 69 - protein, 54 aa, Artificial Sequence
  Synthetic
PEPEKYLQELCKKYYEGKLSQQEAVEEIRKYARKWGLEAWKLVWYFMQCVKRDI

SEQ ID NO: 70 - protein, 54 aa, Artificial Sequence
  Synthetic
MEPEKFLKELCKAYYEGKLSQQEAVEEIRSYAMKWGLEAWMLIWYFMQCVKRDI

SEQ ID NO: 71 - protein, 54 aa, Artificial Sequence
  Synthetic
MEPEEFLLELCKAYYEGKLSQIEAVEEIRHYARSFGLEAWQLIWYFMQCVKRDI

SEQ ID NO: 72 - protein, 54 aa, Artificial Sequence
  Synthetic
PEPEKFLSELCKAYYEGKLSQIEAVEEIRSYARSWGLEAWKLIWYFMQCVKRDI

SEQ ID NO: 73 - protein, 54 aa, Artificial Sequence
  Synthetic
PEPEKFLAELCKAYYEGKLSQPEAVEEIRSYARKWGLEAWKLVWYFMLCVKRDI

SEQ ID NO: 74 - protein, 54 aa, Artificial Sequence
  Synthetic
PEPEQFLTELCKKYYEGKLSQPEAVEEIRKYARKWGLEAWKLIWYFMQCVKRDI

SEQ ID NO: 75 - protein, 54 aa, Artificial Sequence; identical to SEQ ID NO: 10
  Synthetic

SEQ ID NO: 76 - protein, 53 aa, Artificial Sequence
  Synthetic
NLWQIFYQLSTILKRTGDPTAKKLLKALAEALKKGDEKALKELAKKATKYIRS

SEQ ID NO: 77 - protein, 43 aa, Artificial Sequence
  Synthetic
PEELRRRVEHFLRQGVERWMVMWMLIQQGFDNKEVWKVLQEVL

SEQ ID NO: 78 - protein, 59 aa, Artificial Sequence
  Synthetic
ELWFLMALVIAACLWAWKASNVEEAEEALWWALVMAREANMRQLEEFVKRMREEVRRHL

SEQ ID NO: 79 - protein, 43 aa, Artificial Sequence
  Synthetic
DVVVLLGLVIVIPERWRLWLIVVIAARVMGLRVEVHGHVIIVI

SEQ ID NO: 80 - protein, 43 aa, Artificial Sequence
  Synthetic
TVVIINGVPFWFEFLWELFIFALLMALALGLKIEFHGELIEVK

SEQ ID NO: 81 - protein, 64 aa, Artificial Sequence
  Synthetic
TPARELVRELVALALLIAVLRNDIVRLTLDVQGFKITVDVDAHWEAFVRILLLAEILVLEWLKG

SEQ ID NO: 82 - protein, 64 aa, Artificial Sequence
  Synthetic
DWRELMLVWMALWWIWWAFLAGVPLIVEVVVNGLHFRVIVDRDPTTNKRALDIMLWVWEWLATL

SEQ ID NO: 83 - protein, 48 aa, Artificial Sequence
  Synthetic
GRWELLVLAYLALLLGAAEAVWRLLELAKKLGDEMAFRWILELWERAL

SEQ ID NO: 84 - protein, 26 aa, Artificial Sequence
  Synthetic
  MISC_FEATURE (2)..(2): X is A,C,E,F,G,H,I,K,LM,N,Q,R,S,T,V,W, or Y
  MISC_FEATURE (3)..(3): X is A,E,G,I,K,Q,R,S,T, or V
  MISC_FEATURE (5)..(5): X is A,C,D,E,F,G,H,I,K,L,M,N,Q,R,S,T,V,W, or Y
  MISC_FEATURE (6)..(7): X is A,C,D,E,F,G,H,I,K,L,M,N,P,Q,R,S,T,V,W,  or Y
  MISC_FEATURE (8)..(8): X is A,C,D,E,F,G,H,I,K,L,M,N,Q,R,S,T,V,W, or Y
  MISC_FEATURE (10)..(10): X is A,C,E,F,H,I,K,L,M,N,Q,R,S,T,V,W, or Y
  MISC_FEATURE (11)..(11): X is A,C,D,E,F,G,H,I,K,L,M,N,P,Q,R,S,T,V,W,  or Y
  MISC_FEATURE (12)..(12): X is A,E,G,H,M,N,Q,S, or T
  MISC_FEATURE (13)..(13): X is A,C,D,E,F,G,H,I,K,L,M,N,P,Q,R,S,T,V,W,  or Y
  MISC_FEATURE (14)..(14): X is A,C,D,E,F,G,H,K,L,M,N,P,Q,R,S,T,V,W, or Y
  MISC_FEATURE (17)..(17): X is A,E,H,I,K,L,M,Q,V, or Y
  MISC_FEATURE (18)..(18): X is A,C,G, or S
  MISC_FEATURE (19)..(19): X is D,F,H,N,Q,W, or Y
  MISC_FEATURE (20)..(20): X is F,W, or Y
  MISC_FEATURE (21)..(21): X is A,C,E,F,H,I,K,L,M,Q,S,T,V,W, or Y
  MISC_FEATURE (22)..(22): X is A,C,F,H,I,L,M,Q,T,V,W, or Y
  MISC_FEATURE (23)..(23): X is A,C,D,G,H,K,M,N,Q,R,S,T,V,W, or Y
  MISC_FEATURE (24)..(24): X is A,C,D,E,F,G,H,I,K,L,M,N,Q,R,S,T,V,W, or Y
  MISC_FEATURE (25)..(26): X is A,C,D,E,F,G,H,I,K,L,M,N,P,Q,R,S,T,V,W,  or Y
CXXCXXXXCXXXXXCWXXXXXXXXXX

SEQ ID NO: 85 - protein, 26 aa, Artificial Sequence
  Synthetic
  MISC_FEATURE (2)..(2): X is R or T
  MISC_FEATURE (3)..(3): X is I, K or V
  MISC_FEATURE (5)..(5): X is A,C,G,H,I,K,L,M,N,Q,R,S,T,V,W, or Y
  MISC_FEATURE (6)..(6): X is A,C,D,E,G,H,K,L,M,N,Q,R,S,T,W, or Y
  MISC_FEATURE (7)..(7): X is A,C,D,E,F,G,H,K,M,N,Q,R,,S,T,W, or Y
  MISC_FEATURE (8)..(8): X is A,C,F,H,I,K,L,M,R,T,V,W, or Y
  MISC_FEATURE (10)..(10): X is C, I, or V
  MISC_FEATURE (11)..(11): X is A,C,D,E,H,I,K,L,M,N,Q,R,S,T,V, or Y
  MISC_FEATURE (12)..(12): X is A or M
  MISC_FEATURE (13)..(13): X is A,C,D,E,H,I,K,L,M,N,P,Q,R,S,T,V, or Y
  MISC_FEATURE (14)..(14): X is A,C,E,F,H,K,L,M,Q,R,S,T,W, or Y
  MISC_FEATURE (17)..(17): X is E, M or Q
  MISC_FEATURE (18)..(18): X is A, C or G
  MISC_FEATURE (19)..(19): X is F, W or Y
  MISC_FEATURE (20)..(20): X is  W or Y
  MISC_FEATURE (21)..(21): X is C,I,L, or M
  MISC_FEATURE (22)..(22): X is I,L,M, or W
  MISC_FEATURE (23)..(23): X is A,N, or S
  MISC_FEATURE (24)..(24): X is A,C,E,F,H,I,K,L,M,N,Q,R,S,T,V,W, or Y
  MISC_FEATURE (25)..(25): X is A,C,F,H,I,L,M,Q,V,W, or Y
  MISC_FEATURE (26)..(26): X is A,C,E,F,G,H,I,K,L,M,N,Q,R,S,T,V,W, or Y
CXXCXXXXCXXXXXCWXXXXXXXXXX

SEQ ID NO: 86 - protein, 26 aa, Artificial Sequence
  Synthetic
  MISC_FEATURE (1)..(1): X is C or Y
  MISC_FEATURE (2)..(2): X is A,C,D,E,F,G,H,I,K,L,M,N,Q,R,S,T,V,W, or Y
  MISC_FEATURE (3)..(3): X is A,C,D,E,F,G,H,I,K,L,M,N,P,Q,R,S,T,V,W, or Y
  MISC_FEATURE (4)..(4): X is A,C,D,E,F,G,H,I,K,L,M,N,P,Q,R,S,T,V,W, or Y
  MISC_FEATURE (5)..(5): X is C or V
  MISC_FEATURE (6)..(6): X is A,D,E,G,H,K,L,M,N,P,Q,R,S,T,W, or Y
  MISC_FEATURE (7)..(7): X is A,C,D,E,F,G,H,I,K,L,M,N,P,Q,R,S,T,V,W, or Y
  MISC_FEATURE (8)..(8): X is A,C,D,E,F,G,H,I,K,L,M,N,P,Q,R,S,T,V,W, or Y
  MISC_FEATURE (9)..(9): X is A,C,D,E,F,G,H,I,K,L,M,N,P,Q,R,S,T,V,W, or Y
  MISC_FEATURE (10)..(10): X is A,F,I,K,L,M,R,V,W, or Y
  MISC_FEATURE (11)..(11): X is A,C,D,E,F,G,H,I,K,L,M,N,Q,R,S,T,V,W, or Y
  MISC_FEATURE (12)..(12): X is A,F,G,L,M,S,W, or Y
  MISC_FEATURE (13)..(13): X is A,C,D,E,F,G,H,I,K,L,M,N,P,Q,R,S,T,V,W, or Y
  MISC_FEATURE (14)..(14): X is A,C,D,E,F,H,K,L,M,N,P,Q,R,S,V,W, or Y
  MISC_FEATURE (15)..(15): X is A,G,H,I,K,L,M,N,P,Q,R,S,T,V,W, or Y
  MISC_FEATURE (17)..(17): X is E,H,I,K,M, or Q
  MISC_FEATURE (19)..(19): X is D,E,F,H,I,M,V,W, or Y
  MISC_FEATURE (20)..(20): X is F, W, or Y
  MISC_FEATURE (21)..(21): X is A,F,H,I,L,Q,T,V,W, or Y
  MISC_FEATURE (22)..(22): X is A,C,D,E,F,G,H,I,K,L,M,N,Q,R,S,T,V,W, or Y
  MISC_FEATURE (23)..(23): X is A,D,E,F,G,H,K,M,N,Q,R,S,T,V,W, or Y
  MISC_FEATURE (24)..(24): X is A,C,D,E,F,H,K,L,M,N,Q,R,S,T,V,W, or Y
  MISC_FEATURE (25)..(25): X is A,C,D,E,F,G,H,I,K,L,M,N,Q,R,S,T,V,W, or Y
  MISC_FEATURE (26)..(26): X is C,G,H,S, or T
XXXXXXXXXXXXXXXWXCXXXXXXXX

SEQ ID NO: 87 - protein, 26 aa, Artificial Sequence
  Synthetic
  MISC_FEATURE (2)..(2): X is A,C,E,F,G,H,I,K,L,M,N,Q,R,S,T,V,W, or Y
  MISC_FEATURE (3)..(3): X is A,I,L,T, or V
  MISC_FEATURE (4)..(4): X is A,C,E,F,H,I,K,L,M,N,Q,R,S,T,V,W, or Y
  MISC_FEATURE (7)..(7): X is A,C,D,E,F,G,H,I,K,L,M,N,P,Q,R,S,T,V, or  Y
  MISC_FEATURE (8)..(8): X is A,C,D,E,F,G,H,I,K,L,M,N,Q,R,S,T,V,W, or Y
  MISC_FEATURE (9)..(9): X is A,E,H,I,L,M,N,Q,S,T,V, or Y
  MISC_FEATURE (11)..(11): X is A,C,E,F,H,I,K,L,M,N,Q,R,S,T,V,W, or Y
  MISC_FEATURE (12)..(12): X is A,F, or G
  MISC_FEATURE (13)..(13): X is A,D,E,G,H,K,N,P,Q,R,S,T,V, or Y
  MISC_FEATURE (14)..(14): X is A,D,E,H,K,M,Q,S, or Y
  MISC_FEATURE (15)..(15): X is H,K,L,M,N,Q, or R
  MISC_FEATURE (17)..(17): X is M or Q
  MISC_FEATURE (19)..(19): X is F,V,W, or Y
  MISC_FEATURE (20)..(20): X is W or Y
  MISC_FEATURE (21)..(21): X is F,I,V,W, or Y
  MISC_FEATURE (22)..(22): X is A,C,E,F,H,I,K,L,M,N,Q,R,S,T,V,W, or Y
  MISC_FEATURE (23)..(23): X is A,H,M,N,Q,R,S,T,W, or Y
  MISC_FEATURE (24)..(24): X is A,K,L,M,Q,R,S, or Y
  MISC_FEATURE (25)..(25): X is C,E,F,H,M,N,Q,S,T,V,W, or Y
CXXXCGXXXFXXXXXWXCXXXXXXXC

SEQ ID NO: 88-100 - skipped (13 entries)

SEQ ID NO: 101 - protein, 53 aa, Artificial Sequence
  Synthetic
  MISC_FEATURE (1)..(1): X is A,D,E,F,H,I,K,M,N,P,Q,R,S, or V
  MISC_FEATURE (2)..(2): X is L,M,R,T, or Y
  MISC_FEATURE (4)..(4): X is I,K,M,P,Q,R,V, or Y
  MISC_FEATURE (5)..(5): X is A,C,F,H,I,K,L,M,P,Q,R,T,V,W, or Y
  MISC_FEATURE (6)..(6): X is F or Y
  MISC_FEATURE (7)..(7): X is W or Y
  MISC_FEATURE (8)..(8): X is C,E,F,I,K,L,M,Q,R,V,W, or Y
  MISC_FEATURE (10)..(10): X is A,D,K,L,N,Q,R,S,T, or V
  MISC_FEATURE (11)..(11): X is A,E,F,H,N,Q,T,W, or Y
  MISC_FEATURE (12)..(12): X is A,C,E,F,H,I,K,L,N,Q,R,S,T,V,W, or Y
  MISC_FEATURE (13)..(13): X is F,H,I,K,L,M,N,Q,R,S,T,W, or Y
  MISC_FEATURE (14)..(14): X is A,D,G,K,N,Q, or T
  MISC_FEATURE (15)..(15): X is N,Q,R, or S
  MISC_FEATURE (16)..(16): X is F,R,T, or Y
  MISC_FEATURE (18)..(18): X is A,D,F,H,K,L,M,N,Q,R,S,W, or Y
  MISC_FEATURE (19)..(19): X is A,E,G,H,I,K,L,N,P,Q,R,S,T, or V
  MISC_FEATURE (20)..(20): X is A,D,E,F,H,I,K,M,Q,R,S,T,V,W, or Y
  MISC_FEATURE (21)..(21): X is A or C
  MISC_FEATURE (22)..(22): X is A,K,Q,S, or V
  MISC_FEATURE (23)..(23): X is D,F,G,K,L,R, or S
  MISC_FEATURE (24)..(24): X is L,M,Q,S, or Y
  MISC_FEATURE (25)..(25): X is A,I,K,L,M,N,Q,R,S,T, or V
  MISC_FEATURE (26)..(26): X is A,K,R,S, or T
  MISC_FEATURE (27)..(27): X is A or D
  MISC_FEATURE (29)..(29): X is A,G,K,N,Q, or R
  MISC_FEATURE (30)..(30): X is A,D,E,H,I,K,L,M,R,S,T, or Y
  MISC_FEATURE (31)..(31): X is A,C,E,F,H,I,K,L,M,N,Q,R,S,T,V, or Y
  MISC_FEATURE (32)..(32): X is A,C,H,I,L,M,N,Q,R,S,T, or V
  MISC_FEATURE (33)..(33): X is A,K,L,R, or T
  MISC_FEATURE (34)..(34): X is A,E,H,I,K,L,R,S,T,W, or Y
  MISC_FEATURE (35)..(35): X is F,G,H,I,M,N,P,Q,R,S,T,V,W, or Y
  MISC_FEATURE (36)..(36): X is D,E,G,P,S,T,V, or Y
  MISC_FEATURE (37)..(37): X is A,D,E,G,H,I,K,L,M,N,P,Q,R,S,T,V, or Y
  MISC_FEATURE (38)..(38): X is A,F,I,K,M,N,P,Q, or R
  MISC_FEATURE (39)..(39): X is A,E,F,H,I,K,L,M,N,P,R,S,T,V,W, or Y
  MISC_FEATURE (40)..(40): X is A,C,E,H,I,K,L,M,N,Q,R,S,T, or V
  MISC_FEATURE (41)..(41): X is A,H,I,K,M,N,Q,R,S, or W
  MISC_FEATURE (42)..(42): X is A,D,E,F,G,H,I,L,M,N,Q,R,S,T,V, or Y
  MISC_FEATURE (43)..(43): X is C,I,L,M,N, or P
  MISC_FEATURE (44)..(44): X is A,E,F,H,I,K,M,N,P,Q,R,S,T,V,W, or Y
  MISC_FEATURE (45)..(45): X is A,D,E,F,G,I,K,L,M,N,Q,R,S,T,V,W, or Y
  MISC_FEATURE (46)..(46): X is K,S,T, or V
  MISC_FEATURE (47)..(47): X is A,C,E,G,I,T, or V
  MISC_FEATURE (48)..(48): X is A,D,E,F,G,H,K,L,M,N,P,Q,T,V, or Y
  MISC_FEATURE (49)..(49): X is A,D,E,F,I,K,L,N,Q,R,S,T,V, or W
  MISC_FEATURE (50)..(50): X is H,I,K,R,W, or Y
  MISC_FEATURE (51)..(51): X is C,I,L,T,V, or Y
  MISC_FEATURE (52)..(52): X is A,D,E,G,H,I,K,L,M,N,Q,R,S,T, or Y
  MISC_FEATURE (53)..(53): X is A,D,F,G,H,I,K,L,M,N,Q,R,S,T,V,W, or Y
XXWXXXXXLXXXXXXXGXXXXXXXXXXLXXXXXXXXXXXXXXXXXXXXXXXXX

SEQ ID NO: 102 - protein, 53 aa, Artificial Sequence
  Synthetic
  MISC_FEATURE (1)..(1): X is D,E,G,H,N,P, or Q
  MISC_FEATURE (2)..(2): X is L or V
  MISC_FEATURE (4)..(4): X is A,E,I,L,M,Q, or V
  MISC_FEATURE (5)..(5): X is C, I or V
  MISC_FEATURE (6)..(6): X is F or Y
  MISC_FEATURE (7)..(7): X is W or Y
  MISC_FEATURE (8)..(8): X is C,E,F,H,L,N,or Q
  MISC_FEATURE (10)..(10): X is D,L,N,Q,S, or V
  MISC_FEATURE (11)..(11): X is A,D,E,N,S, or T
  MISC_FEATURE (12)..(12): X is A,C,E,F,G,I,N,Q,R,S,T,V,W, or Y
  MISC_FEATURE (13)..(13): X is E,I,T,V, or Y
  MISC_FEATURE (14)..(14): X is A,D,G,H,K,N,Q, or T
  MISC_FEATURE (15)..(15): X is A,C,D,E,G,K,N,Q,R,S,T, or V
  MISC_FEATURE (16)..(16): X is A, S or T
  MISC_FEATURE (17)..(17): X is C or G
  MISC_FEATURE (18)..(18): X is C,D,S, or Y
  MISC_FEATURE (19)..(19): X is A,C,E,F,G,I,P,Q, or V
  MISC_FEATURE (20)..(20): X is A,C,D,E,H,I,K,L,N,Q,R,T, or V
  MISC_FEATURE (21)..(21): X is C or A
  MISC_FEATURE (22)..(22): X is G,H,I,K,T, or V
  MISC_FEATURE (23)..(23): X is A,C,I,K,L,N,R,S,T,V, or Y
  MISC_FEATURE (24)..(24): X is F, I or L
  MISC_FEATURE (25)..(25): X is G,I,K,L,M,S,T, or V
  MISC_FEATURE (26)..(26): X is F,G,H,I,K,M,N,T,W, or Y
  MISC_FEATURE (27)..(27): X is A,D,E,I,M,Q, or V
  MISC_FEATURE (29)..(29): X is Q, R or S
  MISC_FEATURE (30)..(30): X is C,D,E,M,Q, or S
  MISC_FEATURE (31)..(31): X is A or C
  MISC_FEATURE (32)..(32): X is C,D,L,N, or V
  MISC_FEATURE (33)..(33): X is A,D,G,H,I,K,L,M,N,Q,R, or S
  MISC_FEATURE (34)..(34): X is A,D,E,F,G,H,I,K,M,N,Q,R,S,W, or Y
  MISC_FEATURE (35)..(35): X is F,G,N,S,W, or Y
  MISC_FEATURE (36)..(36): X is D, E or T
  MISC_FEATURE (37)..(37): X is A,E,H,L,M,N,Q,T, or V
  MISC_FEATURE (38)..(38): X is A,C,D,E,G,I,K,N,P,Q,S,T,W, or Y
  MISC_FEATURE (39)..(39): X is A,D,E,H,I,K,L,N,S,T,V, or Y
  MISC_FEATURE (40)..(40): X is C, L or V
  MISC_FEATURE (41)..(41): X is A,C,D,F,G,H,I,K,L,M,N,S, or W
  MISC_FEATURE (42)..(42): X is C, E or T
  MISC_FEATURE (43)..(43): X is A,F,H,L,W, or Y
  MISC_FEATURE (44)..(44): X is A, E or S
  MISC_FEATURE (45)..(45): X is A,C,D,E,F,I,K,N,Q,R,S,T,V,W, or Y
  MISC_FEATURE (46)..(46): X is C,D,E,H,I,K,L,M,Q,R,S,T,V, or Y
  MISC_FEATURE (47)..(47): X is A, C or V
  MISC_FEATURE (48)..(48): X is A,C,D,E,F,H,I,L,M,N,S,T, or Y
  MISC_FEATURE (49)..(49): X is C,D,F,G,I,K,L,N,R,S,T, or Y
  MISC_FEATURE (50)..(50): X is M, C or Y
  MISC_FEATURE (51)..(51): X is I, C or L
  MISC_FEATURE (52)..(52): X is A,C,G,I,M,N,Q,R,T,W, or Y
  MISC_FEATURE (53)..(53): X is C,D,E,F,G,H,I,K,L,S,T,V, or W
XXWXXXXXLXXXXXXXXXXXXXXXXXXLXXXXXXXXXXXXXXXXXXXXXXXXX

SEQ ID NO: 103 - protein, 53 aa, Artificial Sequence
  Synthetic
  MISC_FEATURE (1)..(1): X is A,E,F,H,I,K,M,N,P,Q,R,S, or V
  MISC_FEATURE (2)..(2): X is L,M,R,T, or Y
  MISC_FEATURE (4)..(4): X is I,K,M,P,Q,R,V, or Y
  MISC_FEATURE (5)..(5): X is A,C,F,H,I,K,L,M,P,Q,R,T,V,W, or Y
  MISC_FEATURE (6)..(6): X is F or Y
  MISC_FEATURE (8)..(8): X is C,I,K,L,M,Q, or V
  MISC_FEATURE (10)..(10): X is A,K,N,Q,R,S, or V
  MISC_FEATURE (11)..(11): X is A,E,F,H,Q,T,W, or Y
  MISC_FEATURE (12)..(12): X is A,C,E,F,H,I,K,L,N,Q,R,T,V,W, or Y
  MISC_FEATURE (13)..(13): X is F,H,I,K,L,M,N,Q,R,S,T,W, or Y
  MISC_FEATURE (14)..(14): X is A,D,G,K,N,Q, or T
  MISC_FEATURE (15)..(15): X is R or S
  MISC_FEATURE (16)..(16): X is F,R,T, or Y
  MISC_FEATURE (18)..(18): X is A,D,F,K,L,M,N,Q,R,S,W, or Y
  MISC_FEATURE (19)..(19): X is A,G,H,I,K,N,P,Q,R,S,T, or V
  MISC_FEATURE (20)..(20): X is A,F,H,I,K,M,Q,R,S,T,V, or Y
  MISC_FEATURE (21)..(21): X is A or C
  MISC_FEATURE (23)..(23): X is K or R
  MISC_FEATURE (24)..(24): X is L,M,Q,S, or Y
  MISC_FEATURE (25)..(25): X is A,I,K,L,M,N,Q,R,S,T, or V
  MISC_FEATURE (26)..(26): X is K or R
  MISC_FEATURE (29)..(29): X is A,G,K,N,Q, or R
  MISC_FEATURE (30)..(30): X is A,D,E,H,I,K,L,M,R,S,T, or Y
  MISC_FEATURE (31)..(31): X is A,C,E,F,H,I,K,L,M,N,Q,R,S,T,V, or Y
  MISC_FEATURE (32)..(32): X is A,C,H,I,L,M,N,Q,R,S,T, or V
  MISC_FEATURE (33)..(33): X is A,K,L,R, or T
  MISC_FEATURE (34)..(34): X is K,L,R, or T
  MISC_FEATURE (35)..(35): X is F,G,H,I,M,N,P,Q,R,S,T,V,W, or Y
  MISC_FEATURE (36)..(36): X is D,E,G,P,S,T,V, or Y
  MISC_FEATURE (37)..(37): X is A,D,E,G,H,I,K,L,M,N,P,Q,R,S,T,V, or Y
  MISC_FEATURE (38)..(38): X is A,K, or P
  MISC_FEATURE (39)..(39): X is A,E,F,H,I,K,L,M,N,P,R,S,T,V,W, or Y
  MISC_FEATURE (40)..(40): X is A,C,E,H,I,K,L,M,N,Q,R,S,T, or V
  MISC_FEATURE (41)..(41): X is A,H,I,K,N,Q,R,S, or W
  MISC_FEATURE (42)..(42): X is A,D,E,F,G,H,I,L,M,N,Q,R,S,T,V, or Y
  MISC_FEATURE (43)..(43): X is C,L,M,N, or P
  MISC_FEATURE (44)..(44): X is A,E,F,H,I,K,M,N,P,Q,R,S,T,V,W, or Y
  MISC_FEATURE (45)..(45): X is A,D,E,G,I,K,L,M,N,Q,R,T,V, or Y
  MISC_FEATURE (46)..(46): X is K,T, or V
  MISC_FEATURE (47)..(47): X is A,C,E,I,T, or V
  MISC_FEATURE (48)..(48): X is A,D,E,F,G,H,K,L,M,N,P,Q,T,V, or Y
  MISC_FEATURE (49)..(49): X is A,D,E,F,I,K,L,N,Q,R,S,T,V, or W
  MISC_FEATURE (50)..(50): X is H,K,R, or Y
  MISC_FEATURE (51)..(51): X is I,L,T,V, or Y
  MISC_FEATURE (52)..(52): X is A,D,E,G,H,I,K,L,M,N,Q,R,S,T, or Y
  MISC_FEATURE (53)..(53): X is D,F,G,H,L,M,Q,R,S,T,V, or Y
XXWXXXYXLXXXXXXXGXXXXKXXXXALXXXXXXXXXXXXXXXXXXXXXXXXX

SEQ ID NO: 104 - protein, 53 aa, Artificial Sequence
  Synthetic
  MISC_FEATURE (1)..(1): X is D,H,N, or Q
  MISC_FEATURE (4)..(4): X is A,E,I,L,M,Q, or V
  MISC_FEATURE (5)..(5): X is C,I, or V
  MISC_FEATURE (6)..(6): X is F or Y
  MISC_FEATURE (7)..(7): X is W or Y
  MISC_FEATURE (8)..(8): X is E,F,H,L,N, or Q
  MISC_FEATURE (11)..(11): X is A,E,S, or T
  MISC_FEATURE (12)..(12): X is C or I
  MISC_FEATURE (13)..(13): X is A,D,G,H,K,N,Q, or T
  MISC_FEATURE (14)..(14): X is A,D,G,H,K,N,Q, or T
  MISC_FEATURE (15)..(15): X is A,C,D,E,G,K,N,Q,R,S,T, or V
  MISC_FEATURE (19)..(19): X is G or P
  MISC_FEATURE (20)..(20): X is A,C,D,E,H,K,N, or T
  MISC_FEATURE (21)..(21): X is A or C
  MISC_FEATURE (22)..(22): X is G,H,I,K, or V
  MISC_FEATURE (23)..(23): X is A,C,I,K,L,N,R,S, or T
  MISC_FEATURE (24)..(24): X is F or L
  MISC_FEATURE (25)..(25): X is G,I,K,M,S,T, or V
  MISC_FEATURE (26)..(26): X is F,I,K,M,N,T, or W
  MISC_FEATURE (27)..(27): X is A,D,E,I,M, or V
  MISC_FEATURE (29)..(29): X is Q,R, or S
  MISC_FEATURE (30)..(30): X is D,E,M,Q, or S
  MISC_FEATURE (31)..(31): X is A or C
  MISC_FEATURE (32)..(32): X is C,D,L, or N
  MISC_FEATURE (33)..(33): X is A,D,G,H,I,K,L,M,N,Q,R, or S
  MISC_FEATURE (34)..(34): X is A,D,E,F,G,H,K,M,N,Q,R,W, or Y
  MISC_FEATURE (35)..(35): X is F,G,S,W, or Y
  MISC_FEATURE (36)..(36): X is D,E, or T
  MISC_FEATURE (37)..(37): X is E or L
  MISC_FEATURE (38)..(38): X is A,D,E,I,K,N,P,Q,S, or T
  MISC_FEATURE (39)..(39): X is A,D,E,H,I,K,N,S,T,V, or Y
  MISC_FEATURE (40)..(40): X is C or L
  MISC_FEATURE (41)..(41): X is A,C,D,F,G,H,I,K,L,M,N,S, or W
  MISC_FEATURE (43)..(43): X is A,F,L, or W
  MISC_FEATURE (44)..(44): X is A,E, or S
  MISC_FEATURE (45)..(45): X is A,C,D,E,F,I,K,N,Q,R,S,T,V,W, or Y
  MISC_FEATURE (46)..(46): X is C,D,E,K,L,M,R, or S
  MISC_FEATURE (47)..(47): X is A,C, or V
  MISC_FEATURE (48)..(48): X is D,E,H,I,M,N,S, or T
  MISC_FEATURE (49)..(49): X is D,G,I,K,L,N,S,T, or Y
  MISC_FEATURE (51)..(51): X is I or L
  MISC_FEATURE (52)..(52): X is A,G,N,Q,R,T,W, or Y
  MISC_FEATURE (53)..(53): X is D,E,G,H,I,S, or T
XLWXXXXXLNXXIXXTGDXXXXXXXXXLXXXXXXXXXXXXXEXXXXXXXYXXX

SEQ ID NO: 105 - protein, 53 aa, Artificial Sequence
  Synthetic
  MISC_FEATURE (1)..(1): X is A,E,H,K,N,Q, or S
  MISC_FEATURE (2)..(2): X is L,R,T, or Y
  MISC_FEATURE (4)..(4): X is K,Q, or Y
  MISC_FEATURE (5)..(5): X is H,I,K,P,Q, or Y
  MISC_FEATURE (8)..(8): X is I,L,M, or V
  MISC_FEATURE (11)..(11): X is A,E,H,Q,T, or Y
  MISC_FEATURE (12)..(12): X is A,C,F,H,I,K,L,N,Q,R,T,V,W, or Y
  MISC_FEATURE (13)..(13): X is I,M,Q,T, or Y
  MISC_FEATURE (18)..(18): X is D or Y
  MISC_FEATURE (19)..(19): X is A,I,K,N,P,R, or T
  MISC_FEATURE (20)..(20): X is H,K,S,T, or Y
  MISC_FEATURE (21)..(21): X is A or C
  MISC_FEATURE (24)..(24): X is L, S or Y
  MISC_FEATURE (25)..(25): X is I, K or V
  MISC_FEATURE (31)..(31): X is A,C,E,F,H,I,K,L,M,N,Q,R,S,T,V, or Y
  MISC_FEATURE (32)..(32): X is A,I,L,M,N,Q,R,S,T,or V
  MISC_FEATURE (33)..(33): X is K,L, or T
  MISC_FEATURE (34)..(34): X is K,L, R, or T
  MISC_FEATURE (35)..(35): X is G,H,R, or Y
  MISC_FEATURE (36)..(36): X is D or G
  MISC_FEATURE (37)..(37): X is A,E,N,P,Q,S, or T
  MISC_FEATURE (38)..(38): X is A,K, or P
  MISC_FEATURE (39)..(39): X is A or F
  MISC_FEATURE (40)..(40): X is A,C,E,H,I,K,L,M,N,Q,R,S,T, or V
  MISC_FEATURE (41)..(41): X is A,H,K,N,Q, or S
  MISC_FEATURE (43)..(43): X is L or P
  MISC_FEATURE (44)..(44): X is A,E,H,K,N,Q,R,T, or V
  MISC_FEATURE (45)..(45): X is D,E,K,M,N,Q,V, or Y
  MISC_FEATURE (47)..(47): X is A or E
  MISC_FEATURE (48)..(48): X is L, M or V
  MISC_FEATURE (49)..(49): X is A, K or R
  MISC_FEATURE (50)..(50): X is H,K,R, or Y
  MISC_FEATURE (51)..(51): X is I or L
  MISC_FEATURE (53)..(53): X is H, S or T
XXWXXFYXLNXXXKRTGXXXXKKXXKALREXXXXXXXXXXXEXXXKXXXXXRX

SEQ ID NO: 106 - protein, 53 aa, Artificial Sequence
  Synthetic
  MISC_FEATURE (1)..(1): X is D or N
  MISC_FEATURE (4)..(4): X is A,I,M,Q, or V
  MISC_FEATURE (5)..(5): X is I or V
  MISC_FEATURE (8)..(8): X is E, F or L
  MISC_FEATURE (14)..(14): X is K or T
  MISC_FEATURE (15)..(15): X is E or R
  MISC_FEATURE (19)..(19): X is G or P
  MISC_FEATURE (20)..(20): X is A, E or T
  MISC_FEATURE (22)..(22): X is G, H or K
  MISC_FEATURE (23)..(23): X is A,C,I,K, or L
  MISC_FEATURE (25)..(25): X is G,K,S,T, or V
  MISC_FEATURE (26)..(26): X is F,I,K,M,N, or W
  MISC_FEATURE (29)..(29): X is R or S
  MISC_FEATURE (30)..(30): X is D or E
  MISC_FEATURE (31)..(31): X is A or C
  MISC_FEATURE (32)..(32): X is L or C
  MISC_FEATURE (33)..(33): X is I, K or M
  MISC_FEATURE (35)..(35): X is G or S
  MISC_FEATURE (37)..(37): X is E or L
  MISC_FEATURE (38)..(38): X is K or P
  MISC_FEATURE (41)..(41): X is C,D,K,N, or W
  MISC_FEATURE (43)..(43): X is A,L, or W
  MISC_FEATURE (45)..(45): X is C,D,F,K,S,W, or Y
  MISC_FEATURE (46)..(46): X is C,K, or M
  MISC_FEATURE (47)..(47): X is A,C, or V
  MISC_FEATURE (48)..(48): X is E or M
  MISC_FEATURE (49)..(49): X is I,K,L, or S
  MISC_FEATURE (52)..(52): X is G,R, or W
XLWXXFYXLNTCIXXTGDXXCXXLXXALXXXXXKXDXXACXEXAXXXXXYIXS

SEQ ID NO: 107 - protein, 53 aa, Artificial Sequence
  Synthetic
  MISC_FEATURE (1)..(1): X is A,D,E,H,N, or Q
  MISC_FEATURE (2)..(2): X is L,M, or T
  MISC_FEATURE (7)..(7): X is W or Y
  MISC_FEATURE (8)..(8): X is C,E,F,I,L,Q,R,V,W, or Y
  MISC_FEATURE (10)..(10): X is A,D,L,N,Q,S,T, or V
  MISC_FEATURE (11)..(11): X is E,N,Q, or T
  MISC_FEATURE (12)..(12): X is C,E,F,H,K,R,S,T,W, or Y
  MISC_FEATURE (14)..(14): X is G,K, or Q
  MISC_FEATURE (15)..(15): X is N,Q, or R
  MISC_FEATURE (16)..(16): X is T or Y
  MISC_FEATURE (18)..(18): X is A,D,H,N,R,S, or Y
  MISC_FEATURE (19)..(19): X is E,H,L,N,P, or T
  MISC_FEATURE (20)..(20): X is A,D,E,F,H,Q,R,T,V, or W
  MISC_FEATURE (22)..(22): X is A,K,Q,S, or V
  MISC_FEATURE (23)..(23): X is D,F,G,K,L, or S
  MISC_FEATURE (26)..(26): X is A,K,S, or T
  MISC_FEATURE (27)..(27): X is A or D
  MISC_FEATURE (33)..(33): X is A or K
  MISC_FEATURE (34)..(34): X is A,E,H,I,K,S,W, or Y
  MISC_FEATURE (35)..(35): X is H or N
  MISC_FEATURE (37)..(37): X is E,G,M,Q, or T
  MISC_FEATURE (38)..(38): X is F,I,K,M,N,Q, or R
  MISC_FEATURE (39)..(39): X is A,I, or L
  MISC_FEATURE (41)..(41): X is K or M
  MISC_FEATURE (42)..(42): X is E,I, or N
  MISC_FEATURE (43)..(43): X is I or L
  MISC_FEATURE (45)..(45): X is D,F,I,K,L,S,V,W, or Y
  MISC_FEATURE (46)..(46): X is K,S, or V
  MISC_FEATURE (47)..(47): X is A or G
  MISC_FEATURE (48)..(48): X is T or G
  MISC_FEATURE (50)..(50): X is I,W, or Y
  MISC_FEATURE (52)..(52): X is L,Q,R,S, or Y
  MISC_FEATURE (53)..(53): X is A,F,I,K,L,M,N,Q,S,V,W, or Y
XXWQIFXXLXXXIXXXGXXXCXXLLXXLQEAVXXXDXXXVXXXAXXXXKXCXX

SEQ ID NO: 108 - protein, 53 aa, Artificial Sequence
  Synthetic
  MISC_FEATURE (1)..(1): X is D,E,G,H,N, or P
  MISC_FEATURE (2)..(2): X is L or V
  MISC_FEATURE (10)..(10): X is D,L,N,Q,S, or V
  MISC_FEATURE (11)..(11): X is D,E,N,S, or T
  MISC_FEATURE (12)..(12): X is A,C,E,F,G,I,N,Q,R,S,T,V,W, or Y
  MISC_FEATURE (13)..(13): X is E,I,T,V, or Y
  MISC_FEATURE (15)..(15): X is G or R
  MISC_FEATURE (16)..(16): X is A,S, or T
  MISC_FEATURE (17)..(17): X is G or C
  MISC_FEATURE (18)..(18): X is C,D,S, or Y
  MISC_FEATURE (19)..(19): X is A,C,E,F,G,I,P,Q, or V
  MISC_FEATURE (20)..(20): X is A,C,I,L,Q,R,T, or V
  MISC_FEATURE (22)..(22): X is K or T
  MISC_FEATURE (23)..(23): X is C,K,T,V, or Y
  MISC_FEATURE (24)..(24): X is I or L
  MISC_FEATURE (26)..(26): X is G,H,K, or Y
  MISC_FEATURE (27)..(27): X is A or Q
  MISC_FEATURE (30)..(30): X is C or E
  MISC_FEATURE (33)..(33): X is K or R
  MISC_FEATURE (34)..(34): X is H,I,K,Q, or S
  MISC_FEATURE (37)..(37): X is A,E,H,M,N,Q,T, or V
  MISC_FEATURE (38)..(38): X is A,C,G,K,Q,W, or Y
  MISC_FEATURE (39)..(39): X is A or L
  MISC_FEATURE (41)..(41): X is H,I, or K
  MISC_FEATURE (42)..(42): X is C, E, or T
  MISC_FEATURE (43)..(43): X is H, L, or Y
  MISC_FEATURE (45)..(45): X is A,C,F,K,S,V, or Y
  MISC_FEATURE (46)..(46): X is C,H,I,K,L,Q,R,S,T,V, or Y
  MISC_FEATURE (48)..(48): X is A,C,F,I,L,M,S,T, or Y
  MISC_FEATURE (49)..(49): X is C,D,F,G,I,K, or R
  MISC_FEATURE (50)..(50): X is C,M, or Y
  MISC_FEATURE (52)..(52): X is C,I,M, or R
  MISC_FEATURE (53)..(53): X is C,F,I,K,L,S,T,V, or W
XXWQIFYCLXXXXKXXXXXXCXXXLXXLQXAVXXNDXXXVXXXAXXAXXXCXX

SEQ ID NO: 109 - skipped

SEQ ID NO: 110 - protein, 53 aa, Artificial Sequence; identical to SEQ ID NO: 76
  Synthetic

SEQ ID NO: 111 - protein, 53 aa, Artificial Sequence
  Synthetic
NLWMIFYLLNTIIKRTGDPTAKKLLKALQEALKKYDEKAIKELAKKAMKYIRS

SEQ ID NO: 112 - protein, 53 aa, Artificial Sequence
  Synthetic
NLWQIFYILNTIIKRTGDPTAKKLKKALREATKKGDEKAMKELAKKAMKYIRS

SEQ ID NO: 113 - protein, 53 aa, Artificial Sequence
  Synthetic
NLWQIFYILNTIHKRTGDPTAKKLPKALREAMKKGDEKAMKELAKKALKYIRS

SEQ ID NO: 114 - protein, 53 aa, Artificial Sequence
  Synthetic
NLWQIFYLLNTIIKRTGDPTAKKLKKALREALKKGDEKAVKELAKKAMKYIRS

SEQ ID NO: 115 - protein, 53 aa, Artificial Sequence
  Synthetic
NLWMIFYLLNTIFKRTGDPTAKKLKKALNEAMKKGDEKAMKELAKKATKYIRS

SEQ ID NO: 116 - protein, 53 aa, Artificial Sequence
  Synthetic
NLWVIFYLLNTIHKRTGDPTAKKLIKALDEAMKKGDEKATKELAKKALKYIRS

SEQ ID NO: 117 - protein, 53 aa, Artificial Sequence
  Synthetic
NLWQIFYMLNTIFKRTGDPTAKKLLKALREATKKGDEKAMKELAKKATKYIRS

SEQ ID NO: 118 - protein, 53 aa, Artificial Sequence
  Synthetic
NLWQIFYVLNTIYKRTGDPTAKKLNKALREALKKHDEKATKELAKKATKYIRS

SEQ ID NO: 119 - protein, 53 aa, Artificial Sequence
  Synthetic
NLWQIFYVLNTIYKRTGDPTAKKLPKALREALKKNDEKATKELAKKAMKYIRS

SEQ ID NO: 120 - protein, 53 aa, Artificial Sequence
  Synthetic
NLWVIFYVLNTIIKRTGDPTAKKLVKALQEAMKKWDEKATKELAKKATKYIRS

SEQ ID NO: 121 - protein, 53 aa, Artificial Sequence
  Synthetic
NLWIIFYQLNTIIKRTGDPTAKKLIKALQEANKKWDEKALKELAKKATKYIRS

SEQ ID NO: 122 - protein, 53 aa, Artificial Sequence
  Synthetic
NLWQIFYVLNTIYKRTGDPTAKKLPKALREAMKKNDEKAIKELAKKAMKYIRS

SEQ ID NO: 123 - protein, 53 aa, Artificial Sequence
  Synthetic
NLWQIFYLLNTCIKRTGDPTCKKLKKALREALKKGDEKAVKELAKKAMKYIRS

SEQ ID NO: 124 - protein, 53 aa, Artificial Sequence
  Synthetic
NLWQIFYLLNTCIKRTGDPTCKKLKKALRECLKKGDEKACKELAKKAMKYIRS

SEQ ID NO: 125 - protein, 53 aa, Artificial Sequence
  Synthetic
NLWQIFYVLNTIYKRTGDPTAKKLNKALRECLKKHDEKACKELAKKATKYIRS

SEQ ID NO: 126 - protein, 53 aa, Artificial Sequence
  Synthetic
NLWQIFYVCNTIYKRTGDPTAKKLCKALREALKKHDEKATKELAKKATKYIRS

SEQ ID NO: 127 - protein, 53 aa, Artificial Sequence
  Synthetic
NLWQIFYVCNTIYKRTGDPTAKKLCKALRECLKKHDEKACKELAKKATKYIRS

SEQ ID NO: 128 - protein, 53 aa, Artificial Sequence
  Synthetic
NLWICFYQLNTIIKRTGDPTAKKLIKALQEANKKWDEKALKELAKKCTKYIRS

SEQ ID NO: 129 - protein, 53 aa, Artificial Sequence
  Synthetic
NLWICFYQLNTIIKRTGDPTAKKLIKALQECNKKWDEKACKELAKKCTKYIRS

SEQ ID NO: 130 - protein, 53 aa, Artificial Sequence
  Synthetic
DLWQIFYLLNTCIKRTGDPTCKKLKKALRECLKKGDEKACKELAKKAMKYIRS

SEQ ID NO: 131 - protein, 53 aa, Artificial Sequence
  Synthetic
NLWIIFYLLNTCIKRTGDPTCKKLKKALRECLKKGDEKACKELAKKAMKYIRS

SEQ ID NO: 132 - protein, 53 aa, Artificial Sequence
  Synthetic
NLWVIFYLLNTCIKRTGDPTCKKLKKALRECLKKGDEKACKELAKKAMKYIRS

SEQ ID NO: 133 - protein, 53 aa, Artificial Sequence
  Synthetic
NLWQIFYLLNTCIKRTGDPTCKKLKKALRECLKKGDEKACKELAYKAMKYIRS

SEQ ID NO: 134 - protein, 53 aa, Artificial Sequence
  Synthetic
DLWIIFYLLNTCIKRTGDPTCKKLKKALRECLKKGDEKACKELAYKAMKYIRS

SEQ ID NO: 135 - protein, 53 aa, Artificial Sequence
  Synthetic
NLWQIFYMLVTIIKRTGDPTAKKLLKALQEAVKKYDEKAVKELAKKALKYIRS

SEQ ID NO: 136 - protein, 53 aa, Artificial Sequence
  Synthetic
NLWQIFYLLSTIWKRTGDPTAKKLLKALQEAVKKNDEKATKELAKKALKYIRS

SEQ ID NO: 137 - protein, 53 aa, Artificial Sequence
  Synthetic
NLWQIFYMLNTIIKRTGDPTAKKLLKALQEATKKWDEKATKELAKKATKYIRS

SEQ ID NO: 138 - protein, 53 aa, Artificial Sequence
  Synthetic
NLWVIFYQLVTIWKRTGDPTAKKLLKALQEAVKKNDEKATKELAKKATKYIRS

SEQ ID NO: 139 - protein, 53 aa, Artificial Sequence
  Synthetic
NLWQIFYMLVTIIKRTGDPTAKKLLKALQEANKKWDEKATKELAKKAMKYIRS

SEQ ID NO: 140 - protein, 53 aa, Artificial Sequence
  Synthetic
NLWQIFYLLQTILKRTGDPTAKKLLKALAEAVKKWDEKAVKELAKKATKYIRS

SEQ ID NO: 141 - protein, 53 aa, Artificial Sequence
  Synthetic
NLWQIFYLLSTIWKRTGDPTAKKLLKALNEALKKHDEKAVKELAKKALKYIRS

SEQ ID NO: 142 - protein, 53 aa, Artificial Sequence
  Synthetic
NLWQIFYVLLTIIKRTGDPTAKKLLKALQEAVKKYDEKALKELAKKAMKYIRS

SEQ ID NO: 143 - protein, 53 aa, Artificial Sequence
  Synthetic
NLWQIFYQLLTIIKRTGDPTAKKLLKALQEAVKKNDEKAVKELAKKATKYIRS

SEQ ID NO: 144 - protein, 53 aa, Artificial Sequence
  Synthetic
NLWQIFYILVTIIKRTGDPTAKKLLKALQEAVKKYDEKATKELAKKALKYIRS

SEQ ID NO: 145 - protein, 53 aa, Artificial Sequence
  Synthetic
NLWQIFYVLSTIIKRTGDPTAKKLLKALQEAVKKNDEKALKELAKKAMKYIRS

SEQ ID NO: 146 - protein, 53 aa, Artificial Sequence
  Synthetic
NLWQIFYVLVTINKRTGDPTAKKLLKALQEAVKKGDEKAVKELAKKATKYIRS

SEQ ID NO: 147 - protein, 53 aa, Artificial Sequence
  Synthetic
NLWQIFYVLVTIIKRTGDPTAKKLLKALQEAVKKGDEKATKELAKKALKYIRS

SEQ ID NO: 148 - protein, 53 aa, Artificial Sequence
  Synthetic
NLWQIFYMLSTIIKRTGDPTAKKLLKALQEATKKWDEKATKELAKKATKYIRS

SEQ ID NO: 149 - protein, 53 aa, Artificial Sequence
  Synthetic
NLWQIFYMLSTIIKRTGDPTAKKLMKALQEATKKWDEKATKELAKKAMKYIRS

SEQ ID NO: 150 - protein, 53 aa, Artificial Sequence
  Synthetic
NLWQIFYCLLTCIKRTGDPTCKKLLKALQEAVKKNDEKAVKELAKKATKYCRS

SEQ ID NO: 151 - protein, 53 aa, Artificial Sequence
  Synthetic
NLWQIFYVLSTCIKRTGDPTCKKLLKALQECVKKNDEKCLKELAKKAMKYIRS

SEQ ID NO: 152 - protein, 53 aa, Artificial Sequence
  Synthetic
NLWQIFYCLSTIIKRTGDPTAKKLLKALQEATKKWDEKATKELAKKATKYCRS

SEQ ID NO: 153 - protein, 53 aa, Artificial Sequence
  Synthetic
ELWQIFYLLNTCIKRTGDPTCKKLIKALRECLKKGDMKACDELAKKAVKYIMS

SEQ ID NO: 154 - protein, 53 aa, Artificial Sequence
  Synthetic
QLWQIFYLLNTCIKRTGDPTCKKLIKALRECLKKGDAKACDELAKKAVKYIMS

SEQ ID NO: 155 - protein, 53 aa, Artificial Sequence
  Synthetic
QLWQIFYLLNTCIKRTCDPTCKKLKKALRECLKKGDAKACDELADKAVKYIMS

SEQ ID NO: 156 - protein, 53 aa, Artificial Sequence
  Synthetic
PLWQIFYLLNTCIKRTGDPTCKKLIKALRECLKKGDPKACAELADKAMKYIMS

SEQ ID NO: 157 - protein, 53 aa, Artificial Sequence
  Synthetic
QLWQIFYLLNTCIKRTGDPTCKKLKKALRECLKKGDAKACKEAADKAVKYIMS

SEQ ID NO: 158 - protein, 53 aa, Artificial Sequence
  Synthetic
ELWQIFYLLNTCIKRTGDPTCKKLIKALRECLKKGDAKACSELADKAMKYIMS

SEQ ID NO: 159 - protein, 53 aa, Artificial Sequence
  Synthetic
ELWQIFYLLNTCIKRTGDPTCKKLIKALRECLKKGDPKACAELAKKAMKYIMS

SEQ ID NO: 160 - protein, 53 aa, Artificial Sequence
  Synthetic
PLWQVFYLLNTCIKRTGDPTCKKLAKALRECLKPGDVKACKEVADKAMDYIRS

SEQ ID NO: 161 - protein, 53 aa, Artificial Sequence
  Synthetic
PLWQVFYLLNTCIKRTGDPTCKKLAKALRECLKKGDLKACNELADKAVKYINS

SEQ ID NO: 162 - protein, 53 aa, Artificial Sequence
  Synthetic
PLWQIFYLLNTCIKRTGDPTCKVLSKALRECLKKGDVKACSELASKAEKYINS

SEQ ID NO: 163 - protein, 53 aa, Artificial Sequence
  Synthetic
ELWQVFYLLNTCIKRTCDPTCKKLAKALRECLKKGDLKACKEDAYKALDYIRS

SEQ ID NO: 164 - protein, 53 aa, Artificial Sequence
  Synthetic
NLWYIFYLLNTCIKRTCDPTCKVLAKALRECLKKGDLKACSELADKAVDYIRS

SEQ ID NO: 165 - protein, 17 aa, Artificial Sequence
  Synthetic
NLWQIFYLLNTCIKRTG

SEQ ID NO: 166 - protein, 21 aa, Artificial Sequence
  Synthetic
NLWQIFYLLNTCIKRTGDPTC

SEQ ID NO: 167 - protein, 22 aa, Artificial Sequence
  Synthetic
NLWQIFYLLNTCIKRTGDPTCK

SEQ ID NO: 168 - protein, 25 aa, Artificial Sequence
  Synthetic
NLWQIFYLLNTCIKRTGDPTCKKLK

SEQ ID NO: 169 - protein, 28 aa, Artificial Sequence
  Synthetic
NLWQIFYLLNTCIKRTGDPTCKKLKKAL

SEQ ID NO: 170 - protein, 32 aa, Artificial Sequence
  Synthetic
NLWQIFYLLNTCIKRTGDPTCKKLKKALRECL

SEQ ID NO: 171 - protein, 33 aa, Artificial Sequence
  Synthetic
NLWQIFYLLNTCIKRTGDPTCKKLKKALRECLK

SEQ ID NO: 172 - protein, 36 aa, Artificial Sequence
  Synthetic
NLWQIFYLLNTCIKRTGDPTCKKLKKALRECLKKGD

SEQ ID NO: 173 - protein, 37 aa, Artificial Sequence
  Synthetic
NLWQIFYLLNTCIKRTGDPTCKKLKKALRECLKKGDE

SEQ ID NO: 174 - protein, 40 aa, Artificial Sequence
  Synthetic
NLWQIFYLLNTCIKRTGDPTCKKLKKALRECLKKGDEKAC

SEQ ID NO: 175 - protein, 41 aa, Artificial Sequence
  Synthetic
NLWQIFYLLNTCIKRTGDPTCKKLKKALRECLKKGDEKACK

SEQ ID NO: 176 - protein, 42 aa, Artificial Sequence
  Synthetic
NLWQIFYLLNTCIKRTGDPTCKKLKKALRECLKKGDEKACKE

SEQ ID NO: 177 - protein, 44 aa, Artificial Sequence
  Synthetic
NLWQIFYLLNTCIKRTGDPTCKKLKKALRECLKKGDEKACKELA

SEQ ID NO: 178 - protein, 45 aa, Artificial Sequence
  Synthetic
NLWQIFYLLNTCIKRTGDPTCKKLKKALRECLKKGDEKACKELAK

SEQ ID NO: 179 - protein, 48 aa, Artificial Sequence
  Synthetic
NLWQIFYLLNTCIKRTGDPTCKKLKKALRECLKKGDEKACKELAKKAM

SEQ ID NO: 180 - protein, 51 aa, Artificial Sequence
  Synthetic
NLWQIFYLLNTCIKRTGDPTCKKLKKALRECLKKGDEKACKELAKKAMKYI

SEQ ID NO: 181 - protein, 23 aa, Artificial Sequence
  Synthetic
CERKEWMERLGHNWMWFYVMNTC

SEQ ID NO: 182 - protein, 23 aa, Artificial Sequence
  Synthetic
CEALEWFERVGKTWMWFYLLNTC

SEQ ID NO: 183 - protein, 23 aa, Artificial Sequence
  Synthetic
CETLEWMKRQGDNWMWFYMMNYC

SEQ ID NO: 184 - protein, 24 aa, Artificial Sequence
  Synthetic
CEEAEKIRRRAQTWEEFYRANQIC

SEQ ID NO: 185 - protein, 24 aa, Artificial Sequence
  Synthetic
CERAHEWAKRVGGWEAFYMANKLC

SEQ ID NO: 186 - protein, 24 aa, Artificial Sequence
  Synthetic
CERAEEERRRARTWEDFYKANKLC

SEQ ID NO: 187 - protein, 24 aa, Artificial Sequence
  Synthetic
CEEARELIRNANGWKDVWKAWKYC

SEQ ID NO: 188 - protein, 32 aa, Artificial Sequence
  Synthetic
ASPELKETCERLERLGMERWLILWCKQRAEEG

SEQ ID NO: 189 - protein, 26 aa, Artificial Sequence
  Synthetic
PDPNRCEDYKRRLHLRWAVLWYCRRF

SEQ ID NO: 190 - protein, 26 aa, Artificial Sequence
  Synthetic
FCITCNNQTFCAEWRWAAWYMCQKAR

SEQ ID NO: 191 - protein, 26 aa, Artificial Sequence
  Synthetic
CRVCDNNFCVDAQWCWAAFYLLQKYK

SEQ ID NO: 192 - protein, 26 aa, Artificial Sequence
  Synthetic
CRVCRNNFCVDAQWCWAAFYMLQKYN

SEQ ID NO: 193 - protein, 26 aa, Artificial Sequence
  Synthetic
CKVKCGPVEFQAQMNWMCFYWRWRYC

SEQ ID NO: 194 - protein, 26 aa, Artificial Sequence
  Synthetic
CRVCMNNFCVDAQMCWMAFYLLNKYN

SEQ ID NO: 195 - protein, 26 aa, Artificial Sequence
  Synthetic
CRVCLNNFCVDAQMCWMAWYLLTKYR

SEQ ID NO: 196 - protein, 26 aa, Artificial Sequence
  Synthetic
FCITCGNETFCSEWRWEAFYLCQKAR

SEQ ID NO: 197 - protein, 26 aa, Artificial Sequence
  Synthetic
CKVKCGPVEFQATARWMCFYWWWKYC

SEQ ID NO: 198 - protein, 26 aa, Artificial Sequence
  Synthetic
FCITCNNQTFCAEWRWMAWYLCWRAR

SEQ ID NO: 199 - protein, 26 aa, Artificial Sequence
  Synthetic
CRVCMNNMCVDARECWMAYYLLNQYN

SEQ ID NO: 200 - protein, 26 aa, Artificial Sequence
  Synthetic
CRVCKNNFCVDAQECWMAYYLLNQYT

SEQ ID NO: 201 - protein, 26 aa, Artificial Sequence
  Synthetic
CRVCKNGFCVDAQECWMAYYLLNQYT

SEQ ID NO: 202 - protein, 26 aa, Artificial Sequence
  Synthetic
CRVCKNGFCVDARECWMAYYLLNQYN

SEQ ID NO: 203 - protein, 26 aa, Artificial Sequence
  Synthetic
CRVCKNKFCVDAVACWMAYYLLNQYT

SEQ ID NO: 204 - protein, 26 aa, Artificial Sequence
  Synthetic
CRVCRNNMCVDARECWMAYYLLNQYT

SEQ ID NO: 205 - protein, 26 aa, Artificial Sequence
  Synthetic
CRVCRNGFCVDAQECWMAYYLLNQYT

SEQ ID NO: 206 - protein, 26 aa, Artificial Sequence
  Synthetic
CRVCRNGFCVDAQECWMAYYLLNQYT

SEQ ID NO: 207 - protein, 26 aa, Artificial Sequence
  Synthetic
CRVCRNNFCVDARECWMAYYLLNQYN

SEQ ID NO: 208 - protein, 26 aa, Artificial Sequence
  Synthetic
CRVCRNNFCVDAVECWMAYYLLNQYT

SEQ ID NO: 209 - protein, 26 aa, Artificial Sequence
  Synthetic
CRVCMNGMCVDAQECWMAYYLLNQYN

SEQ ID NO: 210 - protein, 26 aa, Artificial Sequence
  Synthetic
CRVCMNGMCVDARECWMAYYLLNQYN

SEQ ID NO: 211 - protein, 26 aa, Artificial Sequence
  Synthetic
CRVCMNGMCVDARECWMAYYLLNQYT

SEQ ID NO: 212 - protein, 26 aa, Artificial Sequence
  Synthetic
CRVCMNGMCVDAVECWMAYYLLNQYT

SEQ ID NO: 213 - protein, 26 aa, Artificial Sequence
  Synthetic
CRVCMNGFCVDARECWMAYYLLNQYN

SEQ ID NO: 214 - protein, 26 aa, Artificial Sequence
  Synthetic
CRVCMNGFCVDARECWMAYYLLNQYN

SEQ ID NO: 215 - protein, 26 aa, Artificial Sequence
  Synthetic
CRVCMNGFCVDAVECWMAYYLLNQYT

SEQ ID NO: 216 - protein, 26 aa, Artificial Sequence
  Synthetic
CRVCMNQMCVDAQECWMAYYLLNQYT

SEQ ID NO: 217 - protein, 26 aa, Artificial Sequence
  Synthetic
CKVKCGGVEFEATERWMCYYWLWKYC

SEQ ID NO: 218 - protein, 26 aa, Artificial Sequence
  Synthetic
CKVKCGGVEFEATERWMCFYWFNKYC

SEQ ID NO: 219 - protein, 26 aa, Artificial Sequence
  Synthetic
CKVKCGGVEFEATERWMCFYWLWKYC

SEQ ID NO: 220 - protein, 26 aa, Artificial Sequence
  Synthetic
CKVKCGSVEFEATERWMCYYWAWKYC

SEQ ID NO: 221 - protein, 26 aa, Artificial Sequence
  Synthetic
CKVKCGSVEFEATERWMCYYWLWKYC

SEQ ID NO: 222 - protein, 26 aa, Artificial Sequence
  Synthetic
CKVKCGSVEFEATERWMCYYWLWKYC

SEQ ID NO: 223 - protein, 26 aa, Artificial Sequence
  Synthetic
CKVKCGSVEFEATERWMCYYWLWKYC

SEQ ID NO: 224 - protein, 26 aa, Artificial Sequence
  Synthetic
CKVKCGSVEFEATERWMCYYWLWKYC

SEQ ID NO: 225 - protein, 26 aa, Artificial Sequence
  Synthetic
CKVKCGPVEFEATERWMCYYWLWKYC

SEQ ID NO: 226 - protein, 26 aa, Artificial Sequence
  Synthetic
CKVKCGPVEFEATERWMCFYWLWKYC

SEQ ID NO: 227 - protein, 26 aa, Artificial Sequence
  Synthetic
CKVKCGPVEFEATERWMCFYWYNKYC

SEQ ID NO: 228 - protein, 26 aa, Artificial Sequence
  Synthetic
CKLKCGGVEFEATERWMCYYWWNKYC

(19) United States
(12) Patent Application Publication
(10) Pub. No.: US 2023/0357323 A1
(43) Pub. Date: Nov. 9, 2023

(54) HUMAN IL23 RECEPTOR BINDING POLYPEPTIDES

(71) Applicant: University of Washington, Seattle, WA (US)

(72) Inventors: Stephanie Berger, Seattle, WA (US); Franziska Seeger, Seattle, WA (US); David Baker, Seattle, WA (US); Ta-Yi Yu, Seattle, WA (US)

(21) Appl. No.: 18/003,572

(22) PCT Filed: Jun. 25, 2021
(86) PCT No.: PCT/US2021/039122

§ 371 Date: Dec. 28, 2022

Related U.S. Application Data
(60) Provisional application No. 63/045,381, filed on Jun. 29, 2020.

(51) Int. Cl. C07K 14/00; C12N 15/63; A61P 29/00

(52) U.S. Cl. CPC C07K 14/001; C12N 15/63; A61P 29/00

(57) ABSTRACT

The present disclosure provides human 1L-23R (WL-23R) binding polypeptides, conditionally maximally active SilL˜23R binding proteins, multimers thereof, and methods for using the polypeptides and binding proteins for therapeutic use.

### CROSS-REFERENCE

This application claims priority to U.S. Provisional Patent Application Ser. No. 63/045,381 filed Jun. 29, 2020, incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Jun. 23, 2021 having the file name “20-814-WO-SeqList_ST25.txt” and is 155 kb in size.

### BACKGROUND

IL-23 cytokine plays an important role in both adaptive and innate immunity. IL-23 induces expression of inflammatory cytokines in several lymphocyte subsets, most notably T-helper type 17 (Th17), as well as innate lymphoid cells (ILC) and γ T-cells. Disruption of IL-23-mediated signaling is a genetically and clinically validated therapeutic strategy for the treatment of inflammatory bowel disease (IBD), which includes Crohn's disease and ulcerative colitis. Antibody therapeutics have several limitations. Antibodies have a high cost of manufacturing and generally have moderate to poor stability, requiring a cold chain for manufacture, storage, transport and administration. Antibody therapies must be infused or injected, which can be inconvenient and stressful for patients. Systemic exposure to immunosuppressive antibody therapies such as those common for treatment of autoimmune diseases puts patients at increased risk for tuberculosis reactivation and other serious infections. Thus, as a safety measure, patients can be disqualified from anti-TNF or anti-IL-23 therapies if they test positive for latent tuberculosis or hepatitis B, limiting access to these therapies especially in developing countries where relatively high proportions of the population are positive for HBV or latent TB. Systemic exposure to antibody therapies, which typically have long half-lives in circulation, also promotes generation of anti-drug antibodies (ADA) over time that can neutralize the drug and result in decreased efficacy. Intermittent dosing of anti-TNF antibodies greatly increases the likelihood of developing ADA; if a patient misses a dose due to a lapse in insurance coverage or otherwise, they are at increased risk of the drug losing efficacy.

### SUMMARY

In a first aspect, the disclosure provides human IL-23R (hIL-23R) binding polypeptides, comprising a polypeptide of the general formula X1-X2-X3-X4-X5, wherein X1, X2, X3, and X4 are optional, wherein X5 comprises a polypeptide domain of between 12-20 amino acids in length, and wherein X5 comprises the amino acid sequence of residues 40-47 in SEQ ID NO:1 or 2. In various embodiments, X5 comprises the amino acid sequence of residues 40-47 in the amino acid sequence selected from the group consisting SEQ ID NO: 3-6; X3 is present and comprises a polypeptide domain between 12-20 amino acids in length, and wherein X4 is either absent, or comprises an amino acid linker; X4 is present and comprises an amino acid linker; X3 is present comprises a polypeptide having the amino acid sequence of residues 22-33 in the amino acid sequence selected from the group consisting of SEQ ID NOS:1-6; X5 comprises the amino acid sequence of residues 39-54 in the amino acid sequence selected from the group consisting of SEQ ID NOS:1-6; X3 comprises the amino acid sequence of residues 21-35 in the amino acid sequence selected from the group consisting of SEQ ID NOS:1-6; X4 comprises the amino acid sequence of residues 36-38 in the amino acid sequence selected from the group consisting of SEQ ID NOS:1-6; X1 is present and comprises a polypeptide domain of between 12-20 amino acids in length; X1 comprises the amino acid sequence of residues 1-16 in the amino acid sequence selected from the group consisting of SEQ ID NOS:1-6; X2 is present, and wherein X2 comprises an amino acid linker; and/or X2 comprises the amino acid sequence of residues 17-20 in the amino acid sequence selected from the group consisting of SEQ ID NOS:1-6. In other embodiments, each of X1, X2, X3, X4, and X5 are present. In another embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:10-74. In another embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:69 and 74.

In a second aspect, the disclosure provides hIL-23R binding polypeptides, comprising a polypeptide of the general formula X1-X2-X3-X4-X5, wherein X2, X3, X4, and X5 are optional, wherein X1 comprises a polypeptide domain of between 12-20 amino acids in length, and wherein X1 comprises the amino acid sequence of residues 1-10 in SEQ ID NO:101 or 102. In various embodiments, X1 comprises the amino acid sequence of residues 1-10 in the amino acid sequence selected from the group consisting of SEQ ID NOS:103-108; X3 is present and X3 comprises a polypeptide domain between 12-20 amino acids in length, and wherein X2 is either absent, or comprises an amino acid linker; X3 comprises a polypeptide having the amino acid sequence of residues 25-33 in the amino acid sequence selected from the group consisting of SEQ ID NOS:101-108; X1 comprises the amino acid sequence of residues 1-16 in the amino acid sequence selected from the group consisting of SEQ ID NOS:101-108; X3 comprises the amino acid sequence of residues 19-34 in the amino acid sequence selected from the group consisting of SEQ ID NOS:101-108; X2 comprises the amino acid sequence of residues 17-18 in the amino acid sequence selected from the group consisting of SEQ ID NOS:101-108; X5 is present and comprises a polypeptide domain of between 12-20 amino acids in length; X5 comprises the amino acid sequence of residues 37-53 in the amino acid sequence selected from the group consisting of SEQ ID NOS:101-108; X4 is present, and wherein X4 comprises an amino acid linker; and/or X4 comprises the amino acid sequence of residues 35-36 in the amino acid sequence selected from the group consisting of SEQ ID NOS:101-108. In one embodiment, X1, X2, X3, X4, and X5 are each present. In another embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 110-180. In another embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:160-163.

In a third aspect, the disclosure provides hIL-23R binding polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of a specific polypeptide disclosed herein. In one embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO: 69, 74, and 160-163.

In a fourth aspect, the disclosure provides hIL-23R binding polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:84-87 or 181-228. In one embodiment, the polypeptides comprise a disulfide bond between two cysteine residues in the polypeptide.

In a fifth aspect, the disclosure provides conditionally maximally active hIL-23R binding protein, comprising a first polypeptide component and a second polypeptide component, wherein the first polypeptide component and the second polypeptide component are not present in a fusion protein, wherein
(a) in total the first polypeptide component and the second polypeptide component comprise domains X3 and X5 as defined in any embodiment of the first aspect of the disclosure;
(b) the X3 domain is present in the first polypeptide component and the X5 domain is present in the second polypeptide component;

the first polypeptide component and the second polypeptide component are not maximally active hIL-23R binding protein individually, and wherein the first polypeptide component and the second polypeptide interact to form a maximally active hIL-23R binding protein.

In a sixth aspect, the disclosure provides conditionally maximally active hIL-23R binding proteins, comprising a first polypeptide component and a second polypeptide component, wherein the first polypeptide component and the second polypeptide component are not present in a fusion protein, wherein
(a) in total the first polypeptide component and the second polypeptide component comprise domains X1 and X3 as defined in any embodiment of the second aspect of the disclosure;
(b) the X1 domain is present in the first polypeptide component and the X3 domain is present in the second polypeptide component;

the first polypeptide component and the second polypeptide component are not maximally active hIL-23R binding protein individually, and wherein the first polypeptide component and the second polypeptide non-covalently interact to form a maximally active hIL-23R binding protein.

In a seventh aspect, the disclosure provides polypeptides comprising an X3 domain as defined herein for any embodiment of the first aspect of the disclosure, wherein the polypeptide does not include an X5 domain as defined in any embodiment of the first aspect of the disclosure.

In an eighth aspect, the disclosure provides polypeptides comprising an X3 domain as defined herein for any embodiment of the second aspect of the disclosure, wherein the polypeptide does not include an X1 domain as defined herein for any embodiment of the second aspect of the disclosure.

In various other aspects, the disclosure provides multimers comprising two or more copies of the hIL-23R binding polypeptide, conditionally maximally active hIL-23R binding protein, polypeptide, or polypeptide component of any of embodiment or combination of embodiments disclosed herein; nucleic acid encoding the polypeptide or polypeptide component of any embodiment herein, expression vectors comprising the nucleic acids of the disclosure operatively linked to a suitable control element, cells comprising the polypeptide, polypeptide component, conditionally maximally active hIL-23R binding proteins, multimer, nucleic acid, or expression vector of any embodiment herein, pharmaceutical compositions comprising (a) the polypeptide, polypeptide component, conditionally maximally active hIL-23R binding protein, nucleic acid, expression vector, or cell of any embodiment or combination of embodiments herein; and (b) a pharmaceutically acceptable carrier; and methods for treating a disorder selected from the group consisting of inflammatory bowel disease (IBD) (including but not limited to includes Crohn's disease and ulcerative colitis), psoriasis, atopic dermatitis, rheumatoid arthritis, psoriatic arthritis, osteoarthritis, axial and peripheral spondyloarthritis, ankylosing spondylitis, enthesitis, and tendonitis, comprising administering to a subject in need thereof an amount effective to treat the disorder of the polypeptide, polypeptide component, conditionally maximally active hIL-23R binding protein, nucleic acid, expression vector, cell, or pharmaceutical composition of any embodiment or combination of embodiments herein.

### DESCRIPTION OF THE FIGURES

FIG. 1(A-C). Computational design strategy. Using the crystal structure of the IL-23p19:IL-23R complex (A) as a starting point, we took p19 residue W156 as a hotspot and additional de novo generated hotspots (B) to seed design. Thousands of scaffold proteins were docked at the IL-23R interface such that they incorporated W156 and at least one additional de novo hotspot (C). Scaffold residues within 8 Å of IL-23R were designed to promote high-affinity interaction with IL-23R.

FIG. 2(A-E). Characterization of best computational designs, affinity-matured combinatorial variants, and disulfide-stabilized variants. (A) Binding titration for computational design 23R_A. (B) Temperature and chemical denaturant melts for the best two computational designs. (C) Binding titration for combinatorial variant B08 (based on 23R_B). (D) Temperature and chemical denaturant melts for the highest affinity combinatorial variants. (E) Equilibrium binding constants (K_{D}), on-rates (kₒₙ) and off-rates (k_{off}) for designed proteins as well as the native ligand (IL-23 cytokine) and a competitor molecule (PTG compound C).

FIG. 3. Stability analysis of B08, a representative affinity-matured combinatorial variant and BO4dslf02, a representative disulfide-stabilized variant. (A) Designed proteins were incubated in simulated gastric or intestinal fluids and degradation was assessed by SDS PAGE at 5, 15, 30 and 60 minutes, 4 and 24 hours. (B) Resistance to temperature and chemical denaturant (GuHCl) was assessed by circular dichroism, measuring the helical signature (signal at 222 nm) in the conditions shown normalized to baseline (25° C. and 0 M GuHCl).

FIG. 4(A-B). Proteolytic stability of designed proteins compared to V565-38F, a clinical-stage oral, gut-restricted nanobody targeting TNFa as therapy for IBD. (A) V565-38F appears to be minimally degraded in 1x SIF. (B) After increasing the concentrations of trypsin and chymotrypsin three-fold (3x SIF), V565-38F shows significant degradation after 24 hours SIF digest. Consistent with reported data, V565-38F is efficiently degraded in SGF. Human/rat IL-23R binder rA11dslf02-M1P-R8Q-K35W is similarly stable in SIF and much more stable in SGF than V565-38F. Mouse IL-23R binder mB09dslf01-T48I is more stable in SIF and SGF than B565-38F.

FIG. 5(A-B). Placement of an affinity tag at the amino-versus carboxy terminus of B04dslf02IB impacts proteolytic stability but not potency. (A) B04dslf02IB with N-terminal (6H-B04dsfl02) or C-terminal (B04dslf02-6H) 6-histidine tag were incubated up to 24 hours in SGF or SIF, and degradation assessed by SDS PAGE. (B) Inhibition of IL-23-mediated cell signaling was assessed with an IL-23 reporter assay (Promega).

FIG. 6. Design strategies to generate smaller IL-23R inhibitors with better tissue penetrance.

FIG. 7. Designed IL-23R inhibitor block IL-23-mediated cell signaling in vitro. Cells engineered to express luciferase downstream of IL-23R (Promega) were pre-incubated for 30 minutes with a titration of each inhibitor, then stimulated with 8 ng/mL human IL-23 cytokine for 6 hours. Luciferase substrate was added, luminescence read, and % inhibition of signaling calculated relative to wells with no inhibitor added. IC50 was calculated using linear regression to fit dose response; values are shown above alongside fold increase in potency relative to a competitor molecule PTG compound C.

FIG. 8(A-B). Sequence fitness landscapes representing deep mutational scanning data for 23R_A (A) and 23R_B (B). SSM libraries based on each design were sorted once for high-affinity binding to hIL-23R. The enrichment ratio for each mutation in the sorted pool compared to the naïve pool was calculated and plotted as a heatmap. Values shown are log₂(enrichment ratio).

FIG. 9(A-F). Sequence fitness landscapes representing deep mutational scanning data for A06dslf03 (A and B), BO4dslf02 (C and D), and B11dslf01 (E and F). SSM libraries based on each design were sorted once for high-affinity binding to hIL-23R (left column, FIGS. 9A, C, and E), or cells were pre-incubated with SIF and then sorted for moderate affinity to hIL-23R (right column, FIGS. 9B, D, and F). The enrichment ratio for each mutation in the sorted pool compared to the naïve pool was calculated and plotted as a heatmap. Values shown are log₂(enrichment ratio).

FIG. 10(A-H). Sequence fitness landscapes representing deep mutational scanning data for rA11dslf02 vs. hIL-23R (A and B), rA11dslf02 vs. rIL-23R (C and D), mA03dslf03 vs. mIL-23R (E and F), and mB09dslf01 vs. mIL-23R (G and H). SSM libraries based on each design were sorted once for high-affinity binding to rat or mouse IL-23R as indicated (left column, FIGS. 10A, C, E, and G), or cells were pre-incubated with SIF and then sorted for moderate affinity to rat or mouse IL-23R (right column, FIGS. 10B, D, F, and H). The enrichment ratio for each mutation in the sorted pool compared to the naïve pool was calculated and plotted as a heatmap. Values shown are log₂(enrichment ratio).

FIG. 11(A-D). Sequence fitness landscapes representing deep mutational scanning data for 23R_mini_14 (A and B) and 23R_mini_17 (C and D). SSM libraries based on each design were sorted once for high-affinity binding to hIL-23R as indicated (left column, FIGS. 11A and C), or cells were pre-incubated with SIF and then sorted for high affinity to hIL-23R (right column, FIGS. 11B and D). The enrichment ratio for each mutation in the sorted pool compared to the parent sequence was calculated and plotted as a heatmap. Values shown are log₂(enrichment ratio).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The disclosure provides human IL-23 receptor (hIL-23R) binding polypeptides that can be used for any suitable purpose, including but not limited to treating inflammatory bowel disease (IBD) (including but not limited to includes Crohn's disease and ulcerative colitis), psoriasis, atopic dermatitis, rheumatoid arthritis, psoriatic arthritis, osteoarthritis, axial and peripheral spondyloarthritis, ankylosing spondylitis, enthesitis, and tendonitis.

In a first aspect, the disclosure provides hIL-23R binding polypeptides, comprising a polypeptide of the general formula X1-X2-X3-X4-X5, wherein X1, X2, X3, and X4 are optional, wherein X5 comprises a polypeptide domain of between 12-20 amino acids in length, and wherein X5 comprises the amino acid sequence of residues 40-47 in SEQ ID NO:1 or 2 (see Table 1). Residues 40-47 are present within a polypeptide of between 12-20 amino acids. The additional residues in the X5 domain may be any suitable amino acids.

TABLE 1
23RA genus All

| | (1) Allowable residues: high-affinity | (2) Allowable residues: stability and |
|---|---|---|
| | binding to hIL-23R (without pre- | high-affinity binding to hIL-23R (with |
| | treatment with SIF; includes 23R_A, | pre-treatment with SIF; includes |
| | A06dslf03, rA11dslf02 vs. human | A06dslf03, rA11dslf02 vs. human |
| Sequence | and rat, and mA03dslf03 vs. mouse) | and rat, and mA03dslf03 vs. mouse) |
| position | SEQ ID NO: 1 | SEQ ID NO: 2 |
| 1 | A, C, D, E, F, G, H, I, L, M, N, P, Q, R, | A, C, D, E, G, H, I, K, L, M, N, P, Q, |
| | S, T, V, W, Y | S, T, V |
| 2 | A, D, E, G, H, I, P, T | C, E |
| 3 | A, C, D, E, G, I, L, M, N, P, Q, S, T, V, Y | A, C, D, E, G, K, N, P, S, V, Y |
| 4 | E | C, E |
| 5 | A, C, D, E, F, G, H, I, K, L, M, N, P, Q, R, | A, C, D, E, G, I, K, L, M, N, P, Q |
| | S, T, V, W, Y | |
| 6 | C, F, H, I, K, L, M, T, V, W, Y | C, F, I, L, M, V, W, Y |
| 7 | A, C, I, L, T, V | L, V |
| 8 | A, C, D, E, F, G, H, I, K, L, M, N, Q, R, S, | A, C, D, E, F, G, H, I, K, L, M, N, Q, |
| | T, V, Y | R, S, T, V, W, Y |
| 9 | A, D, E, F, G, H, I, K, L, M, S, W, Y | C, D, E, F, I, L, M, V |
| 10 | A, C, E, F, H, K, L, M, N, Q, R, S, W, Y | L, M, Q |
| 11 | A, C, E, F, H, L, S, T, V, Y | C, I, K, V |
| 12 | A, C, D, E, F, G, H, I, K, L, N, Q, R, S, T | C, D, H, K |
| 13 | C, D, E, F, G, H, I, K, L, M, N, P, Q, R, S, | A, D, E, F, G, H, I, K, L, M, N, Q, R, |
| | T, V, W | T, V, Y |
| 14 | A, F, H, K, L, M, Q, W, Y | V, Y |
| 15 | A, C, E, F, G, H, I, K, L, M, N, Q, R, S, T, | A, C, D, E, F, G, H, M, N, Q, S, T, V, W, Y |
| | V, W, Y | |
| 16 | A, D, E, F, G, K, M, Q, Y | A, C, D, E, G, H, L, M, N, Q, S |
| 17 | A, C, E, F, G, H, I, K, L, M, N, Q, R, S, T, | C, G, H, K, Q, R |
| | V, W, Y | |
| 18 | A, C, F, G, I, K, L, M, N, P, Q, R, T, V, W, Y | A, C, F, K, N, R, S, T, V |
| 19 | A, C, E, H, I, K, L, M, N, Q, R, S, T, V | C, F, I, L, M, T, V |
| 20 | A, D, E, I, K, L, N, P, Q, R, S, T, V, Y | A, C, D, L, M, N, Q, S, T |
| 21 | H, Q, V | H, I, K, Q, V, Y |
| 22 | A, E, I, K, M, N, P, Q, R, S, T, V | C, E, F, H, I, K, L, P, Q, S, T, V |
| 23 | A, D, E, G, H, I, K, L, N, Q, R, S, T, V, Y | C, D, E, F, G, H, K, M, N, Q, R |
| 24 | A, C, G, R | A, D, V |
| 25 | I, L, M, V | M, V |
| 26 | A, E, G, H, I, K, M, S, V | E, F, G, H, L, M, R, S, V, Y |
| 27 | A, C, D, E, F, H, I, K, L, M, N, Q, R, T, V, | C, D, E, H, M, N, R, T |
| | W, Y | |
| 28 | A, C, I, L, N, V | C, I, V |
| 29 | A, Q, R, W, Y | G, H, Q, R, W |
| 30 | H, I, K, L, M, N, P, V | C, D, E, F, I, K, L, M, N, Q, T, V, W |
| 31 | C, F, H, I, L, M, V, W, Y | C, F, I, L, M, V, W, Y |
| 32 | A, P | A, D, H, S |
| 33 | F, H, K, L, M, N, R, V, W | A, F, G, H, I, K, L, N, Q, R, T, V, Y |
| 34 | A, G, H, K, P, Q, R, S, T, Y | A, C, D, E, F, G, H, I, K, L, M, N, Q, S, T, V, W, Y |
| 35 | A, C, D, E, F, G, H, I, K, L, M, N, Q, R, S, | A, C, E, F, G, H, I, K, L, M, N, Q, S, T, V, W, Y |
| | T, V, W, Y | |
| 36 | D, G, H, K, N, Q | C, G, H, N, P, Q |
| 37 | A, E, F, G, H, I, K, L, M, Q, R, S, T, V, W, Y | C, E, G, I, L, Q, T, V, W |
| 38 | E, P, Q | E |
| 39 | A, G, I, L, M, T, V | A, G, I, M, T, V |
| 40 | W | W |
| 41 | F, I, K, M, P, Q, R, W, Y | I, K, L, M, Q, W |
| 42 | F, L, Y | L, M |
| 43 | F, G, I, L, M, T, V | I, V |
| 44 | F, W, Y | Q, W |
| 45 | A, C, D, E, F, G, H, I, L, M, Q, S, T, V, W, Y | C, E, F, H, L, M, Q, V, W, Y |
| 46 | F, Y | F |
| 47 | M | M |
| 48 | E, G, N, Q, S, T | A, F, I, K, L, M, Q, V, Y |
| 49 | C, D, E, F, H, I, L, N, R, T, Y | C |
| 50 | I, K, N, R, V | T, V |
| 51 | D, E, K, N, P, R | A, D, K, N, Q, T |
| 52 | A, D, E, G, H, K, L, N, P, Q, R, S, T, Y | A, G, N, R, S |
| 53 | A, D, E, G, H, K, M, N, P, Q, R, S, T, V, Y | C, D, I, M, Q, Y |
| 54 | A, D, E, G, H, I, K, L, M, N, P, Q, R, S, T, | A, E, I, K, N, P, Q, V |
| | V, W | |

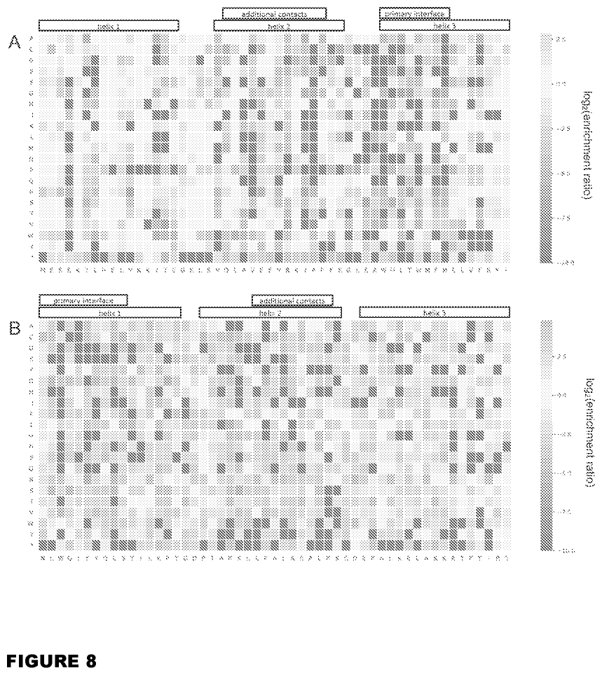

The polypeptides of this embodiment comprise the primary binding interface of the polypeptides of this embodiment for hIL-23R, as described herein (see FIGS. 8-10).

Each of Tables 1-7 includes 2 columns, each representing a different polypeptide of the disclosure by SEQ ID NO. For each Table, the left-hand column provides allowable residues for polypeptides of the disclosure based on mutational analysis of high-affinity binding to hIL-23R without pre-treatment with simulated intestinal fluid (SIF), while the right-hand column provides allowable residues for polypeptides of the disclosure based on mutational analysis of stability and high-affinity binding to hIL-23R with pre-treatment with SIF. The allowable residues were determined based on extensive mutational analysis; see FIGS. 8-10. In one embodiment, X5 comprises the amino acid sequence of residues 40-47 in the amino acid sequence selected from the group consisting SEQ ID NO: 3-6 (See Tables 2-3).

TABLE 2
23RA genus Human only

| | (1) Allowable residues: high-affinity | (2) Allowable residues: stability and |
|---|---|---|
| | binding to hIL-23R (without pre- | high-affinity binding to hIL-23R (with |
| | treatment with SIF; includes 23R_A, | pre-treatment with SIF; includes |
| Sequence | A06dslf03, rA11dslf02 vs. human only) | A06dslf03, rA11dslf02 vs. human only) |
| position | SEQ ID NO: 3 | SEQ ID NO: 4 |
| 1 | A, C, D, E, G, H, I, K, L, M, N, P, Q, S, T, V | A, C, D, E, G, H, I, K, M, N, P, Q, S, T, V |
| 2 | A, C, D, E, G, H, I, P, T | C, E |
| 3 | A, C, D, E, G, I, L, N, P, S, V, Y | A, C, D, E, G, P, V |
| 4 | C, E | C, E |
| 5 | A, C, D, E, F, G, H, I, K, L, M, N, P, Q, R, | A, C, D, E, G, I, K, M, N, P, Q |
| | S, T, V, W, Y | |
| 6 | C, F, I, L, M, V, W, Y | C, F, I, L, M, V, W, Y |
| 7 | A, C, I, L, T, V | L, V |
| 8 | A, C, D, E, F, G, H, I, K, L, M, N, Q, R, S, | A, C, D, E, F, G, H, I, K, L, M, N, Q, R, S, |
| | T, V, W, Y | T, V, W, Y |
| 9 | C, E, G, H, I, L, M, S, Y | C, E, L, M |
| 10 | A, C, E, F, H, K, L, M, N, Q, R, S, W, Y | L, M, Q |
| 11 | C, E, F, H, L, V, Y | C |
| 12 | A, C, F, G, H, K, L, Q, R, S, T | C, K |
| 13 | A, C, D, F, H, I, K, L, M, N, Q, R, S, T, V, | A, H, I, K, M, N, Q, R, T, V |
| | W | |
| 14 | K, Y | Y |
| 15 | A, C, E, H, N, S, T, V, W, Y | A, C, E, H, N, S, T, V, W, Y |
| 16 | A, C, D, E, G, M, N, Q, S | A, C, D, E, G, M, N, Q, S |
| 17 | A, C, E, F, G, H, I, K, L, M, N, Q, R, S, T, | G, H, K |
| | V, W, Y | |
| 18 | , C, F, I, K, L, M, N, P, R, S, T, V, W | A, C, K, N, R, S, T |
| 19 | A, C, F, H, I, K, L, M, N, Q, R, S, T, V | C, F, I, L, T, V |
| 20 | A, C, D, E, I, K, L, M, Q, R, S, T, V | A, C, L, M, Q, S, T |
| 21 | H, I, Q, V | H, I, Q |
| 22 | A, C, E, I, K, N, P, Q, S, T | C, E, I, K, P, Q, S, T |
| 23 | A, C, D, E, F, G, H, I, K, L, N, Q, R, S, T, | C, D, E, F, G, H, K, N, Q, R |
| | V, Y | |
| 24 | A, C, G, R | A |
| 25 | V | V |
| 26 | E, F, G, L, M, S, V, Y | E, F, G, L, M, S, V, Y |
| 27 | A, C, D, E, F, H, I, K, L, M, N, Q, R, T, V, | C, D, E, H, M, N, R |
| | W, Y | |
| 28 | A, C, I, N, V | C, I |
| 29 | Q, R, W, Y | Q, R, W |
| 30 | C, E, H, I, K, L, M, Q, V | C, E, K, L, M, Q, V |
| 31 | C, F, I, L, M, V, W, Y | C, F, I, L, M, V, W, Y |
| 32 | A, S | A, S |
| 33 | F, H, I, K, L, M, N, Q, R, T, V, W, Y | F, H, I, L, N, Q, R, T, Y |
| 34 | A, C, D, E, F, G, H, I, K, L, M, Q, R, S, T, | A, C, D, E, F, G, H, I, K, L, M, Q, T, V, W, |
| | V, W, Y | Y |
| 35 | A, C, E, F, G, H, I, K, L, M, N, Q, R, S, T, | A, C, E, F, G, H, I, K, L, M, N, Q, S, T, V, |
| | V, W, Y | W, Y |
| 36 | G, H | G, H |
| 37 | A, C, E, F, H, L, T, Y | C, I |
| 38 | E, Q | E |
| 39 | A, G | A |
| 40 | W | W |
| 41 | F, I, K, L, M, Q, R, W, Y | I, K, L, M, Q, W |
| 42 | L, M | L, M |
| 43 | I, T, V | I, V |
| 44 | W | W |
| 45 | A, C, E, F, G, H, I, L, M, Q, S, T, V, W, Y | C, E, F, H, L, M, Q, V, W, Y |
| 46 | F, Y | F |
| 47 | M | M |
| 48 | E, F, G, I, K, L, M, N, Q, S, T, Y | F, I, K, L, M, Q, Y |
| 49 | C, D, H, L, R | C |
| 50 | I, K, N, R, V | V |
| 51 | A, K, N, Q, R, T | A, K, N, Q, T |
| 52 | A, D, E, G, H, K, L, N, P, Q, R, S, T | A, G, N, R, S |
| 53 | A, C, D, E, G, H, K, M, N, P, Q, R, S, V, Y | C, D |
| 54 | A, D, E, G, H, I, K, L, M, N, P, Q, R, S, T, | I, N, P, V |
| | V, W | |

TABLE 3
rA11dslf02

| | Allowable residues: high-affinity | Allowable residues: stability and high- |
|---|---|---|
| | binding to hIL-23R (without pre- | affinity binding to hIL-23R (with pre- |
| Sequence | treatment with SIF) | treatment with SIF) |
| position | SEQ ID NO: 5 | SEQ ID NO: 6 |
| 1 | D, E, F, H, I, M, N, T, Y | C, D, E, G, H, I, K, M, N, P, Q, S, T, V |
| 2 | D, E | C, E |
| 3 | P | A, E, P, V |
| 4 | E | C, E |
| 5 | K, Y | A, C, D, I, K, M, N, Q |
| 6 | Y | F, Y |
| 7 | L | L |
| 8 | E, F, K, L, N, R | A, C, D, E, G, I, K, L, M, N, Q, R, S, T, V, W |
| 9 | A, D, E, F, I, K | C, E, L, M |
| 10 | L | L, M, Q |
| 11 | A, C, S, T, V, Y | C |
| 12 | D, E, I, K, R | C, K |
| 13 | E, H, K, N, T | A, H, I, K, M, N, Q, R, T, V |
| 14 | F, K, M, W, Y | Y |
| 15 | A, E, F, G, H, L, N, Q, Y | A, C, E, H, S, T, W, Y |
| 16 | E, F, G, K, M, Y | A, C, D, E, G, M, N, Q, S |
| 17 | A, G, N | G, H, K |
| 18 | G, K, L, Q, R, T | A, C, K, N, R, S, T |
| 19 | L, N | C, F, I, L, T, V |
| 20 | A, E, Q, S, Y | A, C, L, M, Q, S, T |
| 21 | H, Q | H, I, Q |
| 22 | K, P, Q, R | C, E, I, K, P, Q, S, T |
| 23 | E, H | C, D, E, F, G, H, K, N, Q, R |
| 24 | A | A |
| 25 | V | V |
| 26 | E | E, F, G, L, M, S, V, Y |
| 27 | E, I, N | C, D, E, H, M, N, R |
| 28 | I | I |
| 29 | Q, R | Q, R |
| 30 | K | C, E, K, L, M, Q, V |
| 31 | Y | Y |
| 32 | A | A, S |
| 33 | K, R | F, H, I, L, N, Q, R, T, Y |
| 34 | K, P, S, T | A, C, D, F, G, H, I, K, L, M, Q, T, V, W, Y |
| 35 | A, C, D, G, H, K, M, N, Q, R, Y | A, E, K, M, N, Q, T, W |
| 36 | G, H, N | G, H |
| 37 | I, L | C, L |
| 38 | E | E |
| 39 | A | A |
| 40 | W | W |
| 41 | K | K, M, Q |
| 42 | L | L, M |
| 43 | V | I, V |
| 44 | W | W |
| 45 | F, V, W, Y | C, F, V, Y |
| 46 | F | F |
| 47 | M | M |
| 48 | E, Q | F, I, K, L, M, Q, Y |
| 49 | C, D, E, H, I, L, N, T, Y | C |
| 50 | V | V |
| 51 | E, K | A, K, N, Q, T |
| 52 | N, P, R | A, N, R, S |
| 53 | D | C, D |
| 54 | I | I, N, P, V |

In another embodiment, X3 is present, wherein X3 comprises a polypeptide domain between 12-20 amino acids in length, and wherein X4 is either absent, or comprises an amino acid linker. The amino acid linkers of X2 and X4 in all aspects and embodiments of the polypeptides disclosure may be present or absent. When present, the amino acid linker can be of any length or amino acid composition as deemed appropriate for an intended use. In some embodiments, X2 and/or X4 are present and can help contribute to overall stability of the polypeptide. In some embodiments, the linkers may comprise any functional domain(s) as suitable for an intended purpose, including but not limited to albumin (to improve serum half-life), receptor-binding domains, or fluorescent proteins.

In various embodiments, X3 comprises a polypeptide having the amino acid sequence of residues 22-33 in the amino acid sequence selected from the group consisting of SEQ ID NOS:1-6. In these embodiments, the X3 domain is present and provides additional binding contacts between the polypeptides of the disclosure and hIL-23R (see FIGS. 8-10). These additional binding contacts are not required for binding to hIL-23R, but expand the interaction surface permitting higher affinity and specificity in binding. In this embodiment, X3 and X5 may be directly adjacent, or may be connected via an amino acid linker, X4. The linker may be of any suitable length and amino acid composition.

In a further embodiment, X5 comprises the amino acid sequence of residues 39-54 in the amino acid sequence selected from the group consisting of SEQ ID NOS:1-6. In another embodiment, X3 comprises the amino acid sequence of residues 21-35 in the amino acid sequence selected from the group consisting of SEQ ID NOS:1-6. In one embodiment X4 comprises the amino acid sequence of residues 36-38 in the amino acid sequence selected from the group consisting of SEQ ID NOS:1-6.

In a further embodiment, X1 is present and comprises a polypeptide domain of between 12-20 amino acids in length. In this embodiment, X1 may serve to help stabilize the polypeptide in the binding-competent conformation, thereby enhancing binding though not directly interacting with hIL-23R.

In one embodiment, X1 and X3 are both present in the polypeptide. In this embodiment, X1 and X3 may be directly adjacent, or may be connected via an amino acid linker, X2. The linker may be of any suitable length and amino acid composition. In another embodiment, X1, X3, and X4 are all present in the polypeptide. In a further embodiment, X1, X2, X3, and X4 are all present in the polypeptide.

In one embodiment, X1 comprises the amino acid sequence of residues 1-16 in the amino acid sequence selected from the group consisting of SEQ ID NOS:1-6. In a further embodiment, X2 is present and comprises an amino acid linker. In one embodiment, X2 comprises the amino acid sequence of residues 17-20 in the amino acid sequence selected from the group consisting of SEQ ID NOS:1-6.

In another embodiment, X3 is present, and:
(a) X5 comprises the amino acid sequence of residues 40-47 in the amino acid sequence selected from the group consisting SEQ ID NO: 5-6 (See Table 3); and
(b) X3 comprises the amino acid sequence of residues 22-33 in the amino acid sequence selected from the group consisting SEQ ID NO: 5-6 (See Table 3).

In a further embodiment, X3 is present, and:
(a) X5 comprises the amino acid sequence of residues 39-54 in the amino acid sequence selected from the group consisting SEQ ID NO: 5-6 (See Table 3); and
(b) X3 comprises the amino acid sequence of residues 21-35 in the amino acid sequence selected from the group consisting SEQ ID NO: 5-6 (See Table 3).

In another embodiment, X1 is present comprises the amino acid sequence of residues 1-16 in the amino acid sequence selected from the group consisting of SEQ ID NOS:5-6.

In a further embodiment, each of X1, X2, X3, X4, and X5 are present in the polypeptide.

In one embodiment, X5 comprises an alpha helix. In another embodiment, X1, when present, comprises an alpha helix. In a further embodiment, X1, X3, and X5 are all present and each comprises an alpha helix.

In one embodiment of any embodiment herein, X2 and X4 are present, and X2 is 4 amino acids in length and X4 is 3 amino acids in length.

In a further embodiment, each of X1, X2, X3, X4, and X5 are present, and wherein

X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X1 domain present in any of SEQ ID NOS: 10-74;

X2 comprises an amino acid sequence at least 50%, 75%, or 100% identical to the amino acid sequence of an X2 domain present in any of SEQ ID NOS: 10-74,

X3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X3 domain present in any of SEQ ID NOS: 10-74,

X4 comprises an amino acid sequence at least 33%, 66%, or 100% identical to the amino acid sequence of an X4 domain present in any of SEQ ID NOS: 10-74, and

X5 comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X5 domain present in any of SEQ ID NOS: 10-744.

In various embodiments, each of X1, X3, and X5 are each at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of a reference domain present in any of SEQ ID NOS: 10-74. In another embodiment, each of X1, X3, and X5 are each at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of a reference domain present in the same amino acid sequence selected from the group consisting of SEQ ID NOS: 10-74.

In a still further embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:10-74.

X2 and X4 domains are underlined and bolded; X1, X3, and X5 domains are separated by X2 and X4 (i.e.: formula X1-X2-X3-X4-X5)). In all embodiments, 1, 2, 3, 4, 5, 6, 7, 8, 9, 10, or more of the N-terminal amino acids may be deleted from the polypeptide, and thus may be deleted from the reference polypeptide of any one of SEQ ID NOS: 10-74 when considering percent identity. In various other embodiments, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to:
the amino acid sequence of an X5 domain present in a polypeptide selected from the group consisting of SEQ ID NO:10-74;
the amino acid sequence of an X4-X5 domain combination present in a polypeptide selected from the group consisting of SEQ ID NO:10-74:
the amino acid sequence of an X3-X4-X5 domain combination present in a polypeptide selected from the group consisting of SEQ ID NO:10-74: or
the amino acid sequence of an X2-X3-X4-X5 domain combination present in a polypeptide selected from the group consisting of SEQ ID NO:10-74.

>23R_A
(SEQ ID NO: 10)
MESEKYLRELVKKYYEGKLSVQEAVEEVRKYARKKGLEAWMLTWMEMELVKRYI
Enriched combinatorial variants based on 23R_A (A##)
>A01
(SEQ ID NO: 11)
MEPEKYLREKVKKYYEGKLSQPEAVEEIRKYARKKGLEAWKLVWYFMELVKRDI
>A02
(SEQ ID NO: 12)
MEEEKYLRELVKKYYEQKLSHQEAVEIIRKYARKKGLEAWKLVWAFMQLVKRDI
>A03
(SEQ ID NO: 13)
MEEEKYVRELVKKYYEGKLSQPEAVEEIRKYARKKGLEAWKLIWAFMQLVKRDI
>A04
(SEQ ID NO: 14)
MEEEKYVRELVKKYYEGKLSHPEAVEEIRKYARKKGLEAWKLVWAFMQLVKRDI
>A05
(SEQ ID NO: 15)
MEEEKYVREQVKKYYEKKLSQPEAVEIIRKYARKKGLEAWKLIWAFMQLVKRDI
>A06
(SEQ ID NO: 16)
MEPEKYVRELVKKYYEKKLSQPEAVEEIRKYARKKGLEAWMLVWHEMQLVKRDI
>A07
(SEQ ID NO: 17)
MEEEKYLRELVKKYYEKKLSQPEAVEIIRKYARKKGLEAWKLVWAFMQLVKRDI
>A08
(SEQ ID NO: 18)
MEEEKYVRELVKKYYEKKLSQPEAVEEIRKYARKKGLEAWKLVWAFMELVKRNI
>A09
(SEQ ID NO: 19)
MEEEKYLRELVKKYYEQKLSQPEAVEIIRKYARKKGEEAWYLIWMEMELVKRDI
>A10
(SEQ ID NO: 20)
MEEEKYLREQVKKYYEGKLSVVEAVEEVRKYARKKGLEAWKLIWAFMQLVKRDI
>A11
(SEQ ID NO: 21)
MEEEKYVRELVKKYYEGKLSHQEAVVEIRKYARKKGLEAWKLVWMEMQLVKRNI
>A12
(SEQ ID NO: 22)
MEPEKYVRELVKKYYEQKLSQQEAVEIIRKYARKKGLEAWMLVWAFMQLVKRDI
>A13
(SEQ ID NO: 23)
MEPEKYVREKVKKYYEGKLSQPEAVEEIRKYARKKGLEAWKLIWHEMQLVKRDI
>A14
(SEQ ID NO: 24)
MEEEKYLRELVKKYYEQKLSQPEAVEIVRKYARKKGLEAWKLIWAFMELVKRYI
Disulfide-stabilized combinatorial variants
>A03dslf03
(SEQ ID NO: 25)
MEEEKYVRELCKKYYEGKLSQPEAVEEIRKYARKKGLEAWKLIWAFMQCVKRDI
>A03dslf04
(SEQ ID NO: 26)
MEEEKCVRELCKKYYEGKLSQPEAVEEIRKCARKKGLEAWKLIWAFMQCVKRDI
>A04dslf01
(SEQ ID NO: 27)
MEEEKCVRELCKKYYEGKLSHPEAVEEIRKCARKKGLEAWKLVWAFMQCVKRDI
>A05dslf01
(SEQ ID NO: 28)
MEEEKCVREQCKKYYEKKLSQPEAVEIIRKCARKKGLEAWKLIWAFMQCVKRDI
>A06dslf03
(SEQ ID NO: 29)
MEPEKCVRELCKKYYEKKLSQPEAVEEIRKCARKKGLEAWMLVWHEMQCVKRDI
>A08dslf03
(SEQ ID NO: 30)
MEEEKYVRELCKKYYEKKLSQPEAVEEIRKYARKKGLEAWKLVWAFMECVKRNI
>A11dslf01
(SEQ ID NO: 31)
MEEEKYVRELCKKYYEGKLSHQEAVVEIRKYARKKGLEAWKLVWMEMQCVKRNI
>A11dslf02
(SEQ ID NO: 32)
MEEEKCVRELCKKYYEGKLSHQEAVVEIRKCARKKGLEAWKLVWMFMQCVKRNI
Combinatorial variants enriched for binding to mouse (″r″ prefix) IL-23R (″m″ prefix)
>mA01
(SEQ ID NO: 33)
MEPEKYVREQVKKYYEQKLSHQEAVEEVRKYARKKGLEAWKLTWYFMQLVKREI
>mA02
(SEQ ID NO: 34)
MEEEKYVRELVKKYYEQKLSHPEAVEEIRKYARKKGLEAWKLVWYFMQLVKRNI
>mA03
(SEQ ID NO: 35)
MEEEKYVRELVKKYYEGKLSHPEAVEEIRKYARKKGEEAWKLVWYFMQLVKRDI
>rA01
(SEQ ID NO: 36)
MEPEKYVRELVKKYYEKKLSQPEAVEEMRKYARKKGLEAWKLVWHEMQLVKREI
>rA02
(SEQ ID NO: 37)
MEPEKYLRELVKKYYEKKLSQQEAVEEIRKYARKKGLEAWKLVWYFMQLVKREI
>rA03
(SEQ ID NO: 38)
MEPEKYLRELVKKYYEKKLSQPEAVEIIRKYARKKGLEAWKLVWYFMELVKREI
>rA04
(SEQ ID NO: 39)
MEPEKYLRELVKKYYEQKLSQQEAVEEIRKYARKKGLEAWKLIWYFMELVKRDI
>rA05
(SEQ ID NO: 40)
MEPEKYLREMVKKYYEKKLSQQEAVEEIRKYARKKGLEAWKLVWYJMELVKRYI
>rA06
(SEQ ID NO: 41)
MEEEKYVRELVKKYYEQKLSQQEAVEEIRKYARKKGLEAWKLVWYEMELVKRNI
>rA07
(SEQ ID NO: 42)
MEPEKYLRELVKKYYEQKLSQQEAVEIIRKYARKKGLEAWKLIWYFMQLVKRNI
>rA08
(SEQ ID NO: 43)
MEPEKYVRELVKKYYEGKLSQPEAVEEIRKYARKKGLEAWKLVWYFMQLVKRNI
>rA09
(SEQ ID NO: 44)
MEPEKYLRELVKKYYEGKLSQQEAVEEIRKYARKKGLEAWKLIWYFMELVKREI
>rA10
(SEQ ID NO: 45)
MEEEKYVREQVKKYYEGKLSQQEAVEIIRKYARKKGLEAWKLIWYFMQLVKRDI
>rA11
(SEQ ID NO: 46)
MEPEKYLRELVKKYYEGKLSQQEAVEEIRKYARKKGLEAWKLVWYFMQLVKRDI
Disulfide-stabilized combinatorial variants enriched for (″m″ prefix) and rat
(″r″ prefix) IL-23R binding to mouse
>mA01dslf01
(SEQ ID NO: 47)
MEPEKYVREQCKKYYEQKLSHQEAVEEVRKYARKKGLEAWKLTWYFMQCVKREI
>mA03dslf01
(SEQ ID NO: 48)
MEEEKCVRELVKKYYEGKLSHPEAVEEIRKCARKKGEEAWKLVWYFMQLVKRDI
>mA03dslf02
(SEQ ID NO: 49)
MEEEKYVRELCKKYYEGKLSHPEAVEEIRKYARKKGEEAWKLVWYFMQCVKRDI
>mA03dslf03
(SEQ ID NO: 50)
MEEEKCVRELCKKYYEGKLSHPEAVEEIRKCARKKGEEAWKLVWYFMQCVKRDI
>rA01dslf01
(SEQ ID NO: 51)
MEPEKCVRELVKKYYEKKLSQPEAVEEMRKCARKKGLEAWKLVWHEMQLVKREI
>rA01dslf02
(SEQ ID NO: 52)
MEPEKYVRELCKKYYEKKLSQPEAVEEMRKYARKKGLEAWKLVWHEMQCVKREI
>rA01dslf03
(SEQ ID NO: 53)
MEPEKYVRECCKKYYEKKLSQPECVEEMRKYARKKGLEAWKLVWHEMQCVKREI
>rA01dslf04
(SEQ ID NO: 54)
MEPEKCVRELCKKYYEKKLSQPEAVEEMRKCARKKGLEAWKLVWHEMQCVKREI
>rA04dslf01
(SEQ ID NO: 55)
MEPEKCLRELVKKYYEQKLSQQEAVEEIRKCARKKGLEAWKLIWYEMELVKRDI
>rA04dslf02
(SEQ ID NO: 56)
MEPEKYLRELCKKYYEQKLSQQEAVEEIRKYARKKGLEAWKLIWYFMECVKRDI
>rA05dslf01
(SEQ ID NO: 57)
MEPEKCLREMVKKYYEKKLSQQEAVEEIRKCARKKGLEAWKLVWYFMELVKRYI
>rA07dslf01
(SEQ ID NO: 58)
MEPEKYLRELCKKYYEQKLSQQEAVEIIRKYARKKGLEAWKLIWYFMQCVKRNI
>rA08dslf01
(SEQ ID NO: 59)
MEPEKCVRELVKKYYEGKLSQPEAVEEIRKCARKKGLEAWKLVWYFMQLVKRNI
>rA11dslf01
(SEQ ID NO: 60)
MEPEKCLRELVKKYYEGKLSQQEAVEEIRKCARKKGLEAWKLVWYFMQLVKRDI
>rA11dslf02
(SEQ ID NO: 61)
MEPEKYLRELCKKYYEGKLSQQEAVEEIRKYARKKGLEAWKLVWYFMQCVKRDI
>rA11dslf03
(SEQ ID NO: 62)
MEPEKCLRELCKKYYEGKLSQQEAVEEIRKCARKKGLEAWKLVWYFMQCVKRDI
Variants selected manually from SSM data enriched for stability and affinity to
human IL-23R
>rA11dslf02_M1P
(SEQ ID NO: 63)
PEPEKYLRELCKKYYEGKLSQQEAVEEIRKYARKKGLEAWKLVWYFMQCVKRDI
>rA11dslf02_M1T
(SEQ ID NO: 64)
TEPEKYLRELCKKYYEGKLSQQEAVEEIRKYARKKGLEAWKLVWYFMQCVKRDI
>rA11dslf02_R8Q
(SEQ ID NO: 65)
MEPEKYLQELCKKYYEGKLSQQEAVEEIRKYARKKGLEAWKLVWYFMQCVKRDI
>rA11dslf02_C11V
(SEQ ID NO: 66)
MEPEKYLRELVKKYYEGKLSQQEAVEEIRKYARKKGLEAWKLVWYFMQCVKRDI
>rA11dslf02_K35W
(SEQ ID NO: 67)
MEPEKYLRELCKKYYEGKLSQQEAVEEIRKYARKWGLEAWKLVWYFMQCVKRDI
>rA11dslf02_Y45C_C49A
(SEQ ID NO: 68)
MEPEKYLRELCKKYYEGKLSQQEAVEEIRKYARKKGLEAWKLVWCEMQAVKRDI
>rA11dslf02_M1P_R8Q_K35W
(SEQ ID NO: 69)
PEPEKYLQELCKKYYEGKLSQQEAVEEIRKYARKWGLEAWKLVWYFMQCVKRDI
Combinatorial variants based on rA11dslf02 enriched for SIF stability and affinity to
human IL-23R
>rA11dslf02A
(SEQ ID NO: 70)
MEPEKFLKELCKAYYEGKLSQQEAVEEIRSYAMKWGLEAWMLIWYFMQCVKRDI
>rA11dslf02B
(SEQ ID NO: 71)
MEPEEFLLELCKAYYEGKLSQIEAVEEIRHYARSFGLEAWQLIWYFMQCVKRDI
>rA11dslf02C
(SEQ ID NO: 72)
PEPEKFLSELCKAYYEGKLSQIEAVEEIRSYARSWGLEAWKLIWYFMQCVKRDI
>rA11dslf02D
(SEQ ID NO: 73)
PEPEKFLAELCKAYYEGKLSQPEAVEEIRSYARKWGLEAWKLVWYFMLCVKRDI
>rA11dslf02E
(SEQ ID NO: 74)
PEPEQFLTELCKKYYEGKLSQPEAVEEIRKYARKWGLEAWKLIWYFMQCVKRDI

In one embodiment, exemplary substitutions relative to the amino acid sequence selected from the group consisting of SEQ ID NO:10-74 are provided in Tables 1-3.

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:69 and 74. In another embodiment, the polypeptide comprises the amino acid sequence of SEQ ID NO:69 or SEQ ID NO:74.

In a second aspect, the disclosure provides hIL-23R binding polypeptides comprising a polypeptide of the general formula X1-X2-X3-X4-X5, wherein X2, X3, X4, and X5 are optional, wherein X1 comprises a polypeptide domain of between 12-20 amino acids in length, and wherein X1 comprises the amino acid sequence of residues 1-10 in SEQ ID NO:101 or 102 (see Table 4).

TABLE 4
23RB allowable residues (All)

| | (1) Allowable residues: high-affinity | (2) Allowable residues: stability |
|---|---|---|
| | binding to hIL-23R (without pre- | and high-affinity binding to hIL- |
| | treatment with SIF; includes 23R_B, | 23R (with pre-treatment with SIF; |
| | B04dslf02, B11dslf01 and | includes B04dslf02, B11dslf01 and |
| Sequence | mB09dslf01) | mB09dslf01) |
| position | SEQ ID NO: 101 | SEQ ID NO: 102 |
| 1 | A, D, E, F, H, I, K, M, N, P, Q, R, S, V | D, E, G, H, N, P, Q |
| 2 | L, M, R, T, Y | L, V |
| 3 | W | W |
| 4 | I, K, M, P, Q, R, V, Y | A, E, I, L, M, Q, V |
| 5 | A, C, F, H, I, K, L, M, P, Q, R, T, V, W, Y | C, I, V |
| 6 | F, Y | F, Y |
| 7 | W, Y | W, Y |
| 8 | C, E, F, I, K, L, M, Q, R, V, W, Y | C, E, F, H, L, N, Q |
| 9 | L | L |
| 10 | A, D, K, L, N, Q, R, S, T, V | D, L, N, Q, S, V |
| 11 | A, E, F, H, N, Q, T, W, Y | A, D, E, N, S, T |
| 12 | A, C, E, F, H, I, K, L, N, Q, R, S, T, V, W, Y | A, C, E, F, G, I, N, Q, R, S, T, V, W, Y |
| 13 | F, H, I, K, L, M, N, Q, R, S, T, W, Y | E, I, T, V, Y |
| 14 | A, D, G, K, N, Q, T | A, D, G, H, K, N, Q, T |
| 15 | N, Q, R, S | A, C, D, E, G, K, N, Q, R, S, T, V |
| 16 | F, R, T, Y | A, S, T |
| 17 | G | C, G |
| 18 | A, D, F, H, K, L, M, N, Q, R, S, W, Y | C, D, S, Y |
| 19 | A, E, G, H, I, K, L, N, P, Q, R, S, T, V | A, C, E, F, G, I, P, Q, V |
| 20 | A, D, E, F, H, I, K, M, Q, R, S, T, V, W, Y | A, C, D, E, H, I, K, L, N, Q, R, T, V |
| 21 | A, C | A, C |
| 22 | A, K, Q, S, V | G, H, I, K, T, V |
| 23 | D, F, G, K, L, R, S | A, C, I, K, L, N, R, S, T, V, Y |
| 24 | L, M, Q, S, Y | F, I, L |
| 25 | A, I, K, L, M, N, Q, R, S, T, V | G, I, K, L, M, S, T, V |
| 26 | A, K, R, S, T | F, G, H, I, K, M, N, T, W, Y |
| 27 | A, D | A, D, E, I, M, Q, V |
| 28 | L | L |
| 29 | A, G, K, N, Q, R | Q, R, S |
| 30 | A, D, E, H, I, K, L, M, R, S, T, Y | C, D, E, M, Q, S |
| 31 | A, C, E, F, H, I, K, L, M, N, Q, R, S, T, V, Y | A, C |
| 32 | A, C, H, I, L, M, N, Q, R, S, T, V | C, D, L, N, V |
| 33 | A, K, L, R, T | A, D, G, H, I, K, L, M, N, Q, R, S |
| 34 | A, E, H, I, K, L, R, S, T, W, Y | A, D, E, F, G, H, I, K, M, N, Q, R, S, W, Y |
| 35 | F, G, H, I, M, N, P, Q, R, S, T, V, W, Y | F, G, N, S, W, Y |
| 36 | D, E, G, P, S, T, V, Y | D, E, T |
| 37 | A, D, E, G, H, I, K, L, M, N, P, Q, R, S, T, V, Y | A, E, H, L, M, N, Q, T, V |
| 38 | A, F, I, K, M, N, P, Q, R | A, C, D, E, G, I, K, N, P, Q, S, T, W, Y |
| 39 | A, E, F, H, I, K, L, M, N, P, R, S, T, V, W, Y | A, D, E, H, I, K, L, N, S, T, V, Y |
| 40 | A, C, E, H, I, K, L, M, N, Q, R, S, T, V | C, L, V |
| 41 | A, H, I, K, M, N, Q, R, S, W | A, C, D, F, G, H, I, K, L, M, N, S, W |
| 42 | A, D, E, F, G, H, I, L, M, N, Q, R, S, T, V, Y | C, E, T |
| 43 | C, I, L, M, N, P | A, F, H, L, W, Y |
| 44 | A, E, F, H, I, K, M, N, P, Q, R, S, T, V, W, Y | A, E, S |
| 45 | A, D, E, F, G, I, K, L, M, N, Q, R, S, T, V, W, Y | A, C, D, E, F, I, K, N, Q, R, S, T, V, W, Y |
| 46 | K, S, T, V | C, D, E, H, I, K, L, M, Q, R, S, T, V, Y |
| 47 | A, C, E, G, I, T, V | A, C, V |
| 48 | A, D, E, F, G, H, K, L, M, N, P, Q, T, V, Y | A, C, D, E, F, H, I, L, M, N, S, T, Y |
| 49 | A, D, E, F, I, K, L, N, Q, R, S, T, V, W | C, D, F, G, I, K, L, N, R, S, T, Y |
| 50 | H, I, K, R, W, Y | C, M, Y |
| 51 | C, I, L, T, V, Y | C, I, L |
| 52 | A, D, E, G, H, I, K, L, M, N, Q, R, S, T, Y | A, C, G, I, M, N, Q, R, T, W, Y |
| 53 | A, D, F, G, H, I, K, L, M, N, Q, R, S, T, V, W, Y | C, D, E, F, G, H, I, K, L, S, T, V, W |

The polypeptides of this embodiment comprise the primary binding interface of the polypeptides of this embodiment for hIL-23R, as described herein (see FIGS. 8-10). Thus, the polypeptides of this embodiment can be used for any of the methods described herein. Residues 1-10 are present within a polypeptide domain of between 12-20 amino acids. The additional residues in the X1 domain may be any suitable amino acids.

In one embodiment, X1 comprises the amino acid sequence of residues 1-10 in the amino acid sequence selected from the group consisting of SEQ ID NOS:103-108 (See Tables 5-7).

TABLE 5
23RB Human only

| | (1) Allowable residues: high-affinity | (2) Allowable residues: stability |
|---|---|---|
| | binding to hIL-23R (without pre- | and high-affinity binding to hIL- |
| | treatment with SIF; includes 23R_B, | 23R (with pre-treatment with SIF; |
| Sequence | B04dslf02 and B11dslf01) | includes B04dslf02 and B11dslf01) |
| position | SEQ ID NO: 103 | SEQ ID NO: 104 |
| 1 | A, E, F, H, I, K, M, N, P, Q, R, S, V | D, H, N, Q |
| 2 | L, M, R, T, Y | L |
| 3 | W | W |
| 4 | I, K, M, P, Q, R, V, Y | A, E, I, L, M, Q, V |
| 5 | A, C, F, H, I, K, L, M, P, Q, R, T, V, W, Y | C, I, V |
| 6 | F, Y | F, Y |
| 7 | Y | W, Y |
| 8 | C, I, K, L, M, Q, V | E, F, H, L, N, Q |
| 9 | L | L |
| 10 | A, K, N, Q, R, S, V | N |
| 11 | A, E, F, H, Q, T, W, Y | A, E, S, T |
| 12 | A, C, E, F, H, I, K, L, N, Q, R, T, V, W, Y | C, I |
| 13 | F, H, I, K, L, M, N, Q, R, S, T, W, Y | I |
| 14 | A, D, G, K, N, Q, T | A, D, G, H, K, N, Q, T |
| 15 | R, S | A, C, D, E, G, K, N, Q, R, S, T, V |
| 16 | F, R, T, Y | T |
| 17 | G | G |
| 18 | A, D, F, K, L, M, N, Q, R, S, W, Y | D |
| 19 | A, G, H, I, K, N, P, Q, R, S, T, V | G, P |
| 20 | A, F, H, I, K, M, Q, R, S, T, V, Y | A, C, D, E, H, K, N, T |
| 21 | A, C | A, C |
| 22 | K | G, H, I, K, V |
| 23 | K, R | A, C, I, K, L, N, R, S, T |
| 24 | L, M, Q, S, Y | F, L |
| 25 | A, I, K, L, M, N, Q, R, S, T, V | G, I, K, M, S, T, V |
| 26 | K, R | F, I, K, M, N, T, W |
| 27 | A | A, D, E, I, M, V |
| 28 | L | L |
| 29 | A, G, K, N, Q, R | Q, R, S |
| 30 | A, D, E, H, I, K, L, M, R, S, T, Y | D, E, M, Q, S |
| 31 | A, C, E, F, H, I, K, L, M, N, Q, R, S, T, V, Y | A, C |
| 32 | A, C, H, I, L, M, N, Q, R, S, T, V | C, D, L, N |
| 33 | A, K, L, R, T | A, D, G, H, I, K, L, M, N, Q, R, S |
| 34 | K, L, R, T | A, D, E, F, G, H, K, M, N, Q, R, W, Y |
| 35 | F, G, H, I, M, N, P, Q, R, S, T, V, W, Y | F, G, S, W, Y |
| 36 | D, E, G, P, S, T, V, Y | D, E, T |
| 37 | A, D, E, G, H, I, K, L, M, N, P, Q, R, S, T, V, Y | E, L |
| 38 | A, K, P | A, D, E, I, K, N, P, Q, S, T |
| 39 | A, E, F, H, I, K, L, M, N, P, R, S, T, V, W, Y | A, D, E, H, I, K, N, S, T, V, Y |
| 40 | A, C, E, H, I, K, L, M, N, Q, R, S, T, V | C, L |
| 41 | A, H, I, K, N, Q, R, S, W | A, C, D, F, G, H, I, K, L, M, N, S, W |
| 42 | A, D, E, F, G, H, I, L, M, N, Q, R, S, T, V, Y | E |
| 43 | C, L, M, N, P | A, F, L, W |
| 44 | A, E, F, H, I, K, M, N, P, Q, R, S, T, V, W, Y | A, E, S |
| , 45 | A, D, E, G, I, K, L, M, N, Q, R, T, V, Y | A, C, D, E, F, I, K, N, Q, R, S, T, V, W, Y |
| 46 | K, T, V | C, D, E, K, L, M, R, S |
| 47 | A, C, E, I, T, V | A, C, V |
| 48 | A, D, E, F, G, H, K, L, M, N, P, Q, T, V, Y | D, E, H, I, M, N, S, T |
| 49 | A, D, E, F, I, K, L, N, Q, R, S, T, V, W | D, G, I, K, L, N, S, T, Y |
| 50 | H, K, R, Y | Y |
| 51 | I, L, T, V, Y | I, L |
| 52 | A, D, E, G, H, I, K, L, M, N, Q, R, S, T, Y | A, G, N, Q, R, T, W, Y |
| 53 | D, F, G, H, L, M, Q, R, S, T, V, Y | D, E, G, H, I, ST |

TABLE 6
B04dslf02

| | (1) Allowable residues: | (2) Allowable residues: stability |
|---|---|---|
| | high-affinity binding to hIL- | and high-affinity binding to hIL- |
| | 23R (without pre- | 23R (with pre- |
| Sequence | treatment with SIF) | treatment with SIF) |
| position | SEQ ID NO: 105 | SEQ ID NO: 106 |
| 1 | A, E, H, K, N, Q, S | D, N |
| 2 | L, R, T, Y | L |
| 3 | W | W |
| 4 | K, Q, Y | A, I, M, Q, V |
| 5 | H, I, K, P, Q, Y | I, V |
| 6 | F | F |
| 7 | Y | Y |
| 8 | I, L, M, V | E, F, L |
| 9 | L | L |
| 10 | N | N |
| 11 | A, E, H, Q, T, Y | T |
| 12 | A, C, E, H, I, K, L, N, Q, R, T, V, W, Y | C |
| 13 | I, M, Q, T, Y | I |
| 14 | K | K, T |
| 15 | R | E, R |
| 16 | T | T |
| 17 | G | G |
| 18 | D, Y | D |
| 19 | A, I, K, N, P, R, T | G, P |
| 20 | H, K, S, T, Y | A, E, T |
| 21 | A, C | C |
| 22 | K | G, H, K |
| 23 | K | A, C, I, K, L |
| 24 | L, S, Y | L |
| 25 | I, K, V | G, K, S, T, V |
| 26 | K | F, I, K, M, N, W |
| 27 | A | A |
| 28 | L | L |
| 29 | R | R, S |
| 30 | E | D, E |
| 31 | A, C, E, F, H, I, K, L, M, N, Q, R, S, T, V, Y | A, C |
| 32 | A, I, L, M, N, Q, R, S, T, V | C, L |
| 33 | K, L, T | I, K, M |
| 34 | K, L, R, T | K |
| 35 | G, H, R, Y | G, S |
| 36 | D, G | D |
| 37 | A, E, N, P, Q, S, T | E, L |
| 38 | A, K, P | K, P |
| 39 | A, F | A |
| 40 | A, C, E, H, I, K, L, M, N, Q, R, S, T, V | C |
| 41 | A, H, K, N, Q, S | C, D, K, N, W |
| 42 | E | E |
| 43 | L, P | A, L, W |
| 44 | A, E, H, K, N, Q, R, T, V | A |
| 45 | D, E, K, M, N, Q, V, Y | C, D, F, K, S, W, Y, |
| 46 | K | C, K, M |
| 47 | A, E | A, C, V |
| 48 | L, M, V | E, M |
| 49 | A, K, R | I, K, L, S |
| 50 | H, K, R, Y | Y |
| 51 | I, L | I |
| 52 | R | G, R, W |
| 53 | H, S, T | S |

TABLE 7
mB09dslfo1

| | Allowable residues: binding to | Allowable residues: binding to |
|---|---|---|
| | mIL-23R without pre- | mIL-23R with pre- |
| Sequence | treatment with SIF | treatment with SIF |
| position | SEQ ID NO: 107 | SEQ ID NO: 108 |
| 1 | A, D, E, H, N, Q | D, E, G, H, N, P |
| 2 | L, M, T | L, V |
| 3 | W | W |
| 4 | Q | Q |
| 5 | I | I |
| 6 | F | F |
| 7 | W, Y | Y |
| 8 | C, E, F, I, L, Q, R, V, W, Y | C |
| 9 | L | L |
| 10 | A, D, L, N, Q, S, T, V | D, L, N, Q, S, V |
| 11 | E, N, Q, T | D, E, N, S, T |
| 12 | C, E, F, H, K, R, S, T, W, Y | A, C, E, F, G, I, N, Q, R, S, T, V, W, Y |
| 13 | I | E, I, T, V, Y |
| 14 | G, K, Q | K |
| 15 | N, Q, R | G, R |
| 16 | T, Y | A, S, T |
| 17 | G | C, G |
| 18 | A, D, H, N, R, S, Y | C, D, S, Y |
| 19 | E, H, L, N, P, T | A, C, E, F, G, I, P, Q, V |
| 20 | A, D, E, F, H, Q, R, T, V, W | A, C, I, L, Q, R, T, V |
| 21 | C | C |
| 22 | A, K, Q, S, V | K, T |
| 23 | D, F, G, K, L, S | C, K, T, V, Y |
| 24 | L | I, L |
| 25 | L | L |
| 26 | A, K, S, T | G, H, K, Y |
| 27 | A, D | A, Q |
| 28 | L | L |
| 29 | Q | Q |
| 30 | E | C, E |
| 31 | A | A |
| 32 | V | V |
| 33 | A, K | K, R |
| 34 | A, E, H, I, K, S, W, Y | H, I, K, Q, S |
| 35 | H, N | N |
| 36 | D | D |
| 37 | E, G, M, Q, T | A, E, H, M, N, Q, T, V |
| 38 | F, I, K, M, N, Q, R | A, C, G, K, Q, W, Y |
| 39 | A, I, L | A, L |
| 40 | V | V |
| 41 | K, M | H, I, K |
| 42 | E, I, N | C, E, T |
| 43 | I, L | H, L, Y |
| 44 | A | A |
| 45 | D, F, I, K, L, S, V, W, Y | A, C, F, K, S, V, Y |
| 46 | K, S, V | C, H, I, K, L, Q, R, S, T, V, Y |
| 47 | A, G | A |
| 48 | G, T | A, C, F, I, L, M, S, T, Y |
| 49 | K | C, D, F, G, I, K, R |
| 50 | I, W, Y | C, M, Y |
| 51 | C | C |
| 52 | L, Q, R, S, Y | C, I, M, R |
| 53 | A, F, I, K, L, M, N, Q, S, V, W, Y | C, F, I, K, L, S, T, V, W |

In another embodiment, X3 is present and comprises a polypeptide domain between 12-20 amino acids in length. In this embodiment, X2 may be either absent, or comprises an amino acid linker. In a further embodiment, X3 comprises a polypeptide having the amino acid sequence of residues 25-33 in the amino acid sequence selected from the group consisting of SEQ ID NOS:101-108. In these embodiments, the X3 domain is present and provides additional binding contacts between the polypeptides of the disclosure and hIL-23R (see FIGS. 8-10). These additional binding contacts are not required for binding to hIL-23R, but expand the interaction surface permitting higher affinity and specificity in binding. In these embodiments, X3 and X5 may be directly adjacent, or may be connected via an amino acid linker; X4. The linker may be of any suitable length and amino acid composition.

In one embodiment, X1 comprises the amino acid sequence of residues 1-16 in the amino acid sequence selected from the group consisting of SEQ ID NOS:101-108.

In a further embodiment, X3 comprises the amino acid sequence of residues 19-34 in the amino acid sequence selected from the group consisting of SEQ ID NOS:101-108.

In another embodiment, X2 comprises the amino acid sequence of residues 17-18 in the amino acid sequence selected from the group consisting of SEQ ID NOS:101-108.

In a further embodiment, X5 is present and comprises a polypeptide domain of between 12-20 amino acids in length. In this embodiment, X5 may serve to help stabilize the polypeptide in the binding-competent conformation, thereby enhancing binding though not directly interacting with hIL-23R.

In one embodiment, X3 and X5 are both present in the polypeptide. In this embodiment, X3 and X5 may be directly adjacent, or may be connected via an amino acid linker, X4. The linker may be of any suitable length and amino acid composition. In another embodiment, X3, X4, and X5 are all present in the polypeptide. In a further embodiment, X2, X3, X4; and X5 are all present in the polypeptide.

In another embodiment, X5 comprises the amino acid sequence of residues 37-53 in the amino acid sequence selected from the group consisting of SEQ ID NOS:101-108. In one embodiment, X4 is present comprises an amino acid linker. In s further embodiment, X4 comprises the amino acid sequence of residues 35-36 in the amino acid sequence selected from the group consisting of SEQ ID NOS:101-108.

In one embodiment, X3 is present, and:
(a) X1 comprises the amino acid sequence of residues 1-10 in the amino acid sequence selected from the group consisting of SEQ ID NOS:105-108 (Tables 6-7)
(b) X3 comprises the amino acid sequence of residues 25-33 in the amino acid sequence selected from the group consisting of SEQ ID NOS:103-108.

In another embodiment, X3 is present, and:
(a) X1 comprises the amino acid sequence of residues 1-16 in the amino acid sequence selected from the group consisting of SEQ ID NOS:105-108 (Tables 6-7)
(b) X3 comprises the amino acid sequence of residues 19-34 in the amino acid sequence selected from the group consisting of SEQ ID NOS:103-108.

In a further embodiment, X5 is present, and wherein X5 comprises the amino acid sequence of residues 27-53 in the amino acid sequence selected from the group consisting of SEQ ID NOS:105-108.

In one embodiment, X1 comprises an alpha helix. In another embodiment, X3, when present, comprises an alpha helix. In a further embodiment, X5, when present, comprises an alpha helix. In another embodiment, X1, X3, and X5 are all present and each comprises an alpha helix.

In another embodiment, X2 and X4 are present, and wherein each is 2 amino acids in length. In a further embodiment, the second amino acid in X2 and X4 is D. In another embodiment, each of X1, X2, X3, X4, and X5 are present, and wherein

X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X1 domain present in any of SEQ ID NO: 110-180;

X2 comprises an amino acid sequence at least 50% or 100% identical to the amino acid sequence of an X2 domain present in any of SEQ ID NO: 110-180,

X3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X3 domain present in any of SEQ ID NO: 110-164, and 166-180,

X4 comprises an amino acid sequence at least 50% or 100% identical to the amino acid sequence of an X4 domain present in any of SEQ ID NO: 110-164, and 172-180, and

X5 comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X5 domain present in any of SEQ ID NO: 110-164, and 173-180.

In various embodiments, each of X1, X3, and X5 are each at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of a reference domain present in .one of SEQ ID NO: 110-180. In another embodiment, each of X1, X3, and X5 are each at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of a reference domain present in the same amino acid sequence selected from the group consisting of SEQ ID NOS: 110-180.

In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 110-180.

X2 and X4 domains are underlined and bolded; X1, X3, and X5 domains are separated by X2 and X4 (i.e.: formula X1-X2-X3-X4-X5). In all embodiments, 1, 2, 3, 4, 5, 6, 7, 8, 9, 10, or more of the C-terminal amino acids may be deleted from the polypeptide, and thus may be deleted from the reference polypeptide of any one of SEQ ID NOS:110-180 when considering percent identity. In various other embodiments, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to:
the amino acid sequence of an X1 domain present in a polypeptide selected from the group consisting of SEQ ID NO: 110-180;
the amino acid sequence of an X1-X2 domain combination present in a polypeptide selected from the group consisting of SEQ ID NO: 110-164, and 166-180;
the amino acid sequence of an X1-X2-X3 domain combination present in a polypeptide selected from the group consisting of SEQ ID NO: 110-164, and 166-180; or
the amino acid sequence of an X1-X2-X3-X4 domain combination present in a polypeptide selected from the group consisting of SEQ ID NO: 110-164, and 173-180.

(SEQ ID NO: 110)
NLWQIFYQLSTILKRTGDPTAKKLLKALAEALKKGDEKALKELAKKATKYIRS
Enriched combinatorial variants based on 23R_B (B##)
>B01
(SEQ ID NO: 111)
NLWMIFYLLNTIIKRTGDPTAKKLLKALQEALKKYDEKAIKELAKKAMKYIRS
>B02
(SEQ ID NO: 112)
NLWQIFYILNTIIKRTGDPTAKKLKKALREATKKGDEKAMKELAKKAMKYIRS
>B03
(SEQ ID NO: 113)
NLWQIFYILNTIHKRTGDPTAKKLPKALREAMKKGDEKAMKELAKKALKYIRS
>B04
(SEQ ID NO: 114)
NLWQIFYLLNTIIKRTGDPTAKKLKKALREALKKGDEKAVKELAKKAMKYIRS
>B05
(SEQ ID NO: 115)
NLWMIFYLLNTIFKRTGDPTAKKLKKALNEAMKKGDEKAMKELAKKATKYIRS
>B06
(SEQ ID NO: 116)
NLWVIFYLLNTIHKRTGDPTAKKLIKALDEAMKKGDEKATKELAKKALKYIRS
>B07
(SEQ ID NO: 117)
NLWQIFYMLNTIFKRTGDPTAKKLLKALREATKKGDEKAMKELAKKATKYIRS
>B08
(SEQ ID NO: 118)
NLWQIFYVLNTIYKRTGDPTAKKLNKALREALKKHDEKATKELAKKATKYIRS
>B09
(SEQ ID NO: 119)
NLWQIFYVLNTIYKRTGDPTAKKLPKALREALKKNDEKATKELAKKAMKYIRS
>B10
(SEQ ID NO: 120)
NLWVIFYVLNTIIKRTGDPTAKKLVKALQEAMKKWDEKATKELAKKATKYIRS
>B11
(SEQ ID NO: 121)
NLWIIFYQLNTIIKRTGDPTAKKLIKALQEANKKWDEKALKELAKKATKYIRS
>B12
(SEQ ID NO: 122)
NLWQIFYVLNTIYKRTGDPTAKKLPKALREAMKKNDEKAIKELAKKAMKYIRS
Disulfide-stabilized combinatorial variants
>B04dslf01
(SEQ ID NO: 123)
NLWQIFYLLNTCIKRTGDPTCKKLKKALREALKKGDEKAVKELAKKAMKYIRS
>B04dslf02
(SEQ ID NO: 124)
NLWQIFYLLNTCIKRTGDPTCKKLKKALRECEKKGDEKACKELAKKAMKYIRS
>B08dslf01
(SEQ ID NO: 125)
NLWQIFYVLNTIYKRTGDPTAKKLNKALRECLKKHDEKACKELAKKATKYIRS
>B08dslf02
(SEQ ID NO: 126)
NLWQIFYVCNTIYKRTGDPTAKKLCKALREALKKHDEKATKELAKKATKYIRS
>B08dslf03
(SEQ ID NO: 127)
NLWQIFYVCNTIYKRTGDPTAKKLCKALRECLKKHDEKACKELAKKATKYIRS
>B11dslf01
(SEQ ID NO: 128)
NLWICFYQLNTIIKRTGDPTAKKLIKALQEANKKWDEKALKELAKKCTKYIRS
>B11dslf02
(SEQ ID NO: 129)
NLWICFYQLNTIIKRTGDPTAKKLIKALQECNKKWDEKACKELAKKCTKYIRS
Variants selected manually from SSM data enriched for stability and affinity to
human IL-23R
>B04dslf02_N1D
(SEQ ID NO: 130)
DLWQIFYLLNTCIKRTGDPTCKKLKKALRECLKKGDEKACKELAKKAMKYIRS
>B04dslf02_Q4I
(SEQ ID NO: 131)
NLWIIFYLLNTCIKRTGDPTCKKLKKALRECLKKGDEKACKELAKKAMKYIRS
>B04dslf02_Q4V
(SEQ ID NO: 132)
NLWVIFYLLNTCIKRTGDPTCKKLKKALRECLKKGDEKACKELAKKAMKYIRS
>B04dslf02_K45Y
(SEQ ID NO: 133)
NLWQIFYLLNTCIKRTGDPTCKKLKKALRECLKKGDEKACKELAYKAMKYIRS
>B04dslf02_N1D_Q4I_K45Y
(SEQ ID NO: 134)
DLWIIFYLLNTCIKRTGDPTCKKLKKALRECLKKGDEKACKELAYKAMKYIRS
Combinatorial variants enriched for binding to mouse (″m″ prefix) and rat (″r″ prefix)
IL-23R
>mB01
(SEQ ID NO: 135)
NLWQIFYMLVTIIKRTGDPTAKKLLKALQEAVKKYDEKAVKELAKKALKYIRS
>mB02
(SEQ ID NO: 136)
NLWQIFYLLSTIWKRTGDPTAKKLLKALQEAVKKNDEKATKELAKKALKYIRS
>mB03
(SEQ ID NO: 137)
NLWQIFYMLNTIIKRTGDPTAKKLLKALQEATKKWDEKATKELAKKATKYIRS
>mB04
(SEQ ID NO: 138)
NLWVIFYQLVTIWKRTGDPTAKKLLKALQEAVKKNDEKATKELAKKATKYIRS
>mB05
(SEQ ID NO: 139)
NLWQIFYMLVTIIKRTGDPTAKKLLKALQEANKKWDEKATKELAKKAMKYIRS
>mB06
(SEQ ID NO: 140)
NLWQIFYLLQTILKRTGDPTAKKLLKALAEAVKKWDEKAVKELAKKATKYIRS
>mB07
(SEQ ID NO: 141)
NLWQIFYLLSTIWKRTGDPTAKKLLKALNEALKKHDEKAVKELAKKALKYIRS
>mB08
(SEQ ID NO: 142)
NLWQIFYVLLTIIKRTGDPTAKKLLKALQEAVKKYDEKALKELAKKAMKYIRS
>mB09
(SEQ ID NO: 143)
NLWQIFYQLLTIIKRTGDPTAKKLLKALQEAVKKNDEKAVKELAKKATKYIRS
>mB10
(SEQ ID NO: 144)
NLWQIFYILVTIIKRTGDPTAKKLLKALQEAVKKYDEKATKELAKKALKYIRS
>mB11
(SEQ ID NO: 145)
NLWQIFYVLSTIIKRTGDPTAKKLLKALQEAVKKNDEKALKELAKKAMKYIRS
>mB12
(SEQ ID NO: 146)
NLWQIFYVLVTINKRTGDPTAKKLLKALQEAVKKGDEKAVKELAKKATKYIRS
>mB13
(SEQ ID NO: 147)
NLWQIFYVLVTIIKRTGDPTAKKLLKALQEAVKKGDEKATKELAKKALKYIRS
>mB14
(SEQ ID NO: 148)
NLWQIFYMLSTIIKRTGDPTAKKLLKALQEATKKWDEKATKELAKKATKYIRS
>mB15
(SEQ ID NO: 149)
NLWQIFYMLSTIIKRTGDPTAKKLMKALQEATKKWDEKATKELAKKAMKYIRS
Disulfide-stabilized combinatorial variants enriched for binding to mouse (″m″ prefix)
and rat (″r″ prefix) IL-23R
>mB09dslf01
(SEQ ID NO: 150)
NLWQIFYCLLTCIKRTGDPTCKKLLKALQEAVKKNDEKAVKELAKKATKYCRS
>mBl1dslf01
(SEQ ID NO: 151)
NLWQIFYVLSTCIKRTGDPTCKKLLKALQECVKKNDEKCLKELAKKAMKYIRS
>mB14dslf01
(SEQ ID NO: 152)
NLWQIFYCLSTIIKRTGDPTAKKLLKALQEATKKWDEKATKELAKKATKYCRS
Combinatorial variants based on B04dslf02 enriched for SIF stability and affinity to
human IL-23R
>B0402SA
(SEQ ID NO: 153)
ELWQIFYLLNTCIKRTGDPTCKKLIKALRECLKKGDMKACDELAKKAVKYIMS
>B0402SB
(SEQ ID NO: 154)
QLWQIFYLLNTCIKRTGDPTCKKLIKALRECLKKGDAKACDELAKKAVKYIMS
>B0402SC
(SEQ ID NO: 155)
QLWQIFYLLNTCIKRTCDPTCKKLKKALRECLKKGDAKACDELADKAVKYIMS
>B0402SD
(SEQ ID NO: 156)
PLWQIFYLLNTCIKRTGDPTCKKLIKALRECLKKGDPKACAELADKAMKYIMS
>B0402SE
(SEQ ID NO: 157)
QLWQIFYLLNTCIKRTGDPTCKKLKKALRECLKKGDAKACKEAADKAVKYIMS
>B0402SF
(SEQ ID NO: 158)
ELWQIFYLLNTCIKRTGDPTCKKLIKALRECLKKGDAKACSELADKAMKYIMS
>B0402SG
(SEQ ID NO: 159)
ELWQIFYLLNTCIKRTGDPTCKKLIKALRECLKKGDPKACAELAKKAMKYIMS
>B0402IA
(SEQ ID NO: 160)
PLWQVFYLLNTCIKRTGDPTCKKLAKALRECLKPGDVKACKEVADKAMDYIRS
>B0402IB
(SEQ ID NO: 161)
PLWQVFYLLNTCIKRTGDPTCKKLAKALRECLKKGDLKACNELADKAVKYINS
>B0402IC
(SEQ ID NO: 162)
PLWQIFYLLNTCIKRTGDPTCKVLSKALRECLKKGDVKACSELASKAEKYINS
>B0402ID
(SEQ ID NO: 163)
ELWQVFYLLNTCIKRTCDPTCKKLAKALRECLKKGDLKACKEDAYKALDYIRS
>B0402IE
(SEQ ID NO: 164)
NLWYIFYLLNTCIKRTCDPTCKVLAKALRECLKKGDLKACSELADKAVDYIRS
Exemplary truncations based on B04dslf02. All truncated sequences showing enrichment
after selection for affinity only or affinity and stability in the first or second
rounds are deemed allowable.
>B04dslf02_trunc01
(SEQ ID NO: 165)
NLWQIFYLLNTCIKRTG
>B04dslf02_trunc02
(SEQ ID NO: 166)
NLWQIFYLLNTCIKRTGDPTC
>B04dslf02_trunc03
(SEQ ID NO: 167)
NLWQIFYLLNTCIKRTGDPTCK
>B04dslf02_trunc04
(SEQ ID NO: 168)
NLWQIFYLLNTCIKRTGDPTCKKLK
>B04dslf02_trunc05
(SEQ ID NO: 169)
NLWQIFYLLNTCIKRTGDPTCKKLKKAL
>B04dslf02_trunc06
(SEQ ID NO: 170)
NLWQIFYLLNTCIKRTGDPTCKKLKKALRECL
>B04dslf02_trunc07
(SEQ ID NO: 171)
NLWQIFYLLNTCIKRTGDPTCKKLKKALRECEK
>B04dslf02_trunc08
(SEQ ID NO: 172)
NLWQIFYLLNTCIKRTGDPTCKKLKKALRECLKKGD
>B04dslf02_trunc09
(SEQ ID NO: 173)
NLWQIFYLLNTCIKRTGDPTCKKLKKALRECLKKGDE
>B04dslf02_trunc10
(SEQ ID NO: 174)
NLWQIFYLLNTCIKRTGDPTCKKLKKALRECLKKGDEKAC
>B04dslf02_trunc11
(SEQ ID NO: 175)
NLWQIFYLLNTCIKRTGDPTCKKLKKALRECEKKGDEKACK
>B04dslf02_trunc12
(SEQ ID NO: 176)
NLWQIFYLLNTCIKRTGDPTCKKLKKALRECLKKGDEKACKE
>B04dslf02_trunc13
(SEQ ID NO: 177)
NLWQIFYLLNTCIKRTGDPTCKKLKKALRECLKKGDEKACKELA
>B04dslf02_trunc14
(SEQ ID NO: 178)
NLWQIFYLLNTCIKRTGDPTCKKLKKALRECLKKGDEKACKELAK
>B04dslf02_trunc15
(SEQ ID NO: 179)
NLWQIFYLLNTCIKRTGDPTCKKLKKALRECLKKGDEKACKELAKKAM
>B04dslf02_trunc16
(SEQ ID NO: 180)
NLWQIFYLLNTCIKRTGDPTCKKLKKALRECLKKGDEKACKELAKKAMKYI

In one embodiment, exemplary substitutions relative to the amino acid sequence selected from the group consisting of SEQ ID NO: 110-180 are provided in Tables 4-7.

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:160-163. In 50 another embodiment, the polypeptide comprises the amino acid sequence selected from SEQ ID NO: 160-163.

In a third aspect, the disclosure provides hIL-23R binding polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of a specific polypeptide disclosed herein. In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NOS:69, 74, and 160-163. In another embodiment, the polypeptide comprises the amino acid sequence selected from SEQ ID NOS: 69, 74, and 160-163.

In a fourth aspect, the disclosure provides hIL-23R binding polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:181-228. In all embodiments, 1, 2, 3, or more of the N-terminal and/or C-terminal amino acids may be deleted from the polypeptide, and thus may be deleted from the reference polypeptide of any one of SEQ ID NOS: 181-228 when considering percent identity.

>23R_mini_01
(SEQ ID NO: 181)
CERKEWMERLGHNWMWFYVMNTC
>23R_mini_02
(SEQ ID NO: 182)
CEALEWFERVGKTWMWFYLLNTC
>23R_mini_03
(SEQ ID NO: 183)
CETLEWMKRQGDNWMWFYMMNYC
>23R_mini_04
(SEQ ID NO: 184)
CEEAEKIRRRAQTWEEFYRANQIC
>23R_mini_05
(SEQ ID NO: 185)
CERAHEWAKRVGGWEAFYMANKLC
>23R_mini_06
(SEQ ID NO: 186)
CERAEEERRRARTWEDFYKANKLC
>23R_mini_07
(SEQ ID NO: 187)
CEEARELIRNANGWKDVWKAWKYC
>23R_mini_08
(SEQ ID NO: 188)
ASPELKETCERLERLGMERWLILWCKQRAEEG
>23R_mini_09
(SEQ ID NO: 189)
PDPNRCEDYKRRLHLRWAVLWYCRRF
>23R_mini_10
(SEQ ID NO: 190)
FCITCNNQTFCAEWRWAAWYMCQKAR
>23R_mini_11
(SEQ ID NO: 191)
CRVCDNNFCVDAQWCWAAFYLLQKYK
>23R_mini_12
(SEQ ID NO: 192)
CRVCRNNFCVDAQWCWAAFYMLQKYN
>23R_mini_13
(SEQ ID NO: 193)
CKVKCGPVEFQAQMNWMCFYWRWRYC
>23R_mini_14
(SEQ ID NO: 194)
CRVCMNNFCVDAQMCWMAFYLLNKYN
>23R_mini_15
(SEQ ID NO: 195)
CRVCLNNFCVDAQMCWMAWYLLTKYR
>23R_mini_16
(SEQ ID NO: 196)
FCITCGNETFCSEWRWEAFYLCQKAR
>23R_mini_17
(SEQ ID NO: 197)
CKVKCGPVEFQATARWMCFYWWWKYC
>23R_mini_18
(SEQ ID NO: 198)
FCITCNNQTFCAEWRWMAWYLCWRAR
>23R_mini_14_C1
(SEQ ID NO: 199)
CRVCMNNMCVDARECWMAYYLLNQYN
>23R_mini_14_C2
(SEQ ID NO: 200)
CRVCKNNFCVDAQECWMAYYLLNQYT
>23R_mini_14_C3
(SEQ ID NO: 201)
CRVCKNGFCVDAQECWMAYYLLNQYT
>23R_mini_14_C4
(SEQ ID NO: 202)
CRVCKNGFCVDARECWMAYYLLNQYN
>23R_mini_14_C5
(SEQ ID NO: 203)
CRVCKNKFCVDAVACWMAYYLLNQYT
>23R_mini_14_C6
(SEQ ID NO: 204)
CRVCRNNMCVDARECWMAYYLLNQYT
>23R_mini_14_C7
(SEQ ID NO: 205)
CRVCRNGFCVDAQECWMAYYLLNQYT
>23R_mini_14_C8
(SEQ ID NO: 206)
CRVCRNGFCVDAQECWMAYYLLNQYT
>23R_mini_14_C9
(SEQ ID NO: 207)
CRVCRNNFCVDARECWMAYYLLNQYN
>23R_mini_14_C10
(SEQ ID NO: 208)
CRVCRNNFCVDAVECWMAYYLLNQYT
>23R_mini_14_C11
(SEQ ID NO: 209)
CRVCMNGMCVDAQECWMAYYLLNQYN
>23R_mini_14_C12
(SEQ ID NO: 210)
CRVCMNGMCVDARECWMAYYLLNQYN
>23R_mini_14_C13
(SEQ ID NO: 211)
CRVCMNGMCVDARECWMAYYLLNQYT
>23R_mini_14_C14
(SEQ ID NO: 212)
CRVCMNGMCVDAVECWMAYYLLNQYT
>23R_mini_14_C15
(SEQ ID NO: 213)
CRVCMNGFCVDARECWMAYYLLNQYN
>23R_mini_14_C16
(SEQ ID NO: 214)
CRVCMNGFCVDARECWMAYYLLNQYN
>23R_mini_14_C17
(SEQ ID NO: 215)
CRVCMNGFCVDAVECWMAYYLLNQYT
>23R_mini_14_C18
(SEQ ID NO: 216)
CRVCMNQMCVDAQECWMAYYLLNQYT
>23R_mini_17_C1
(SEQ ID NO: 217)
CKVKCGGVEFEATERWMCYYWLWKYC
>23R_mini_17_C2
(SEQ ID NO: 218)
CKVKCGGVEFEATERWMCFYWFNKYC
>23R_mini_17_C3
(SEQ ID NO: 219)
CKVKCGGVEFEATERWMCFYWLWKYC
>23R_mini_17_C4
(SEQ ID NO: 220)
CKVKCGSVEFEATERWMCYYWAWKYC
>23R_mini_17_C5
(SEQ ID NO: 221)
CKVKCGSVEFEATERWMCYYWLWKYC
>23R_mini_17_C6
(SEQ ID NO: 222)
CKVKCGSVEFEATERWMCYYWLWKYC
>23R_mini_17_C7
(SEQ ID NO: 223)
CKVKCGSVEFEATERWMCYYWLWKYC
>23R_mini _17_C8
(SEQ ID NO: 224)
CKVKCGSVEFEATERWMCYYWLWKYC
>23R_mini_17_C9
(SEQ ID NO: 225)
CKVKCGPVEFEATERWMCYYWLWKYC
>23R_mini_17_C10
(SEQ ID NO: 226)
CKVKCGPVEFEATERWMCFYWLWKYC
>23R_mini_17_C11
(SEQ ID NO: 227)
CKVKCGPVEFEATERWMCFYWYNKYC
>23R_mini_17_C12
(SEQ ID NO: 228)
CKLKCGGVEFEATERWMCYYWWNKYC

As described in the examples that follow, hIL-23R binding polypeptides of this fourth aspect possess three-dimensional structural elements such that two cysteine residues can be relatively positioned with suitable geometry to form an intramolecular disulfide bond. Thus, in one embodiment the polypeptides of this fourth aspect comprise a disulfide bond between two cysteine residues in the polypeptide.

In one embodiment, allowable substitutions relative to the amino acid sequence selected from the group consisting of SEQ ID NO:194 and 199-216 are provided in Tables 8, and allowable substitutions relative to the amino acid sequence selected from the group consisting of SEQ ID NO:197 and 217-228 are provided in Table 9. Each of Tables 8-9 includes 2 columns. For each Table, the left-hand column provides allowable residues for polypeptides of the disclosure based on mutational analysis of high-affinity binding to hIL-23R (without pre-treatment with simulated intestinal fluid [RF]), while the right-hand column provides allowable residues for polypeptides of the disclosure based on mutational analysis of stability and high-affinity binding to hIL-23R (with pre-treatment with SIF). The allowable residues were determined based on extensive mutational analysis; see the examples that follow.

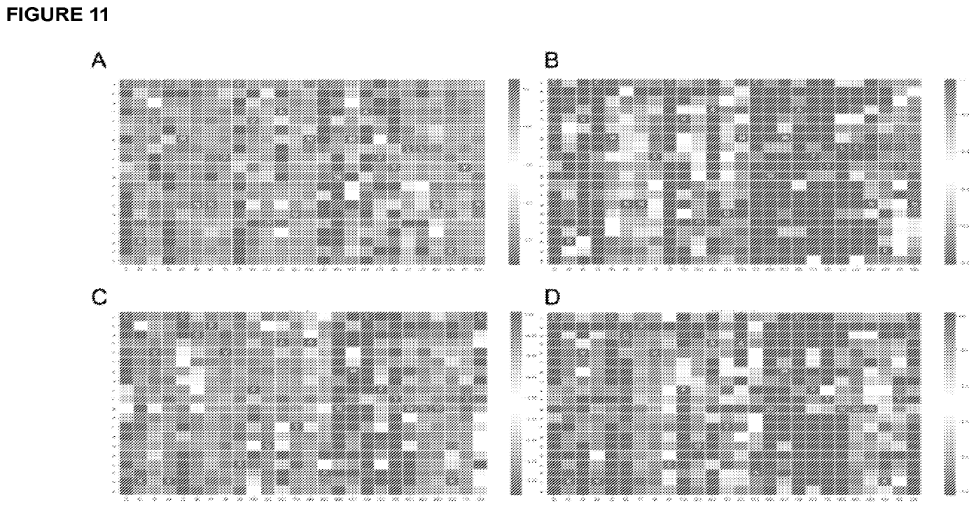

TABLE 8
Allowable residues per position of construct 23R_mini_14, based on the
fitness of single mutants for binding hIL-23R determined during directed evolution,
without (1) or with (2) pre-treatment with simulated intestinal fluid (SIF;
see FIGS. 11A and 11B, respectively). All mutants with at least 2-fold enrichment
in the first selection relative to the naive pool are deemed allowable.

| | Allowable residues: | Allowable residues : |
|---|---|---|
| | binding to hIL-23R | binding to hIL-23R |
| | without pre-treatment | with pre-treatment |
| Sequence | with SIF | with SIF |
| position | (SEQ ID NO: 84) | (SEQ ID NO : 85) |
| 1 | C | C |
| 2 | A, C, E, F, G, H, I, K, LM, N, Q, R, | R, T |
| | S, T, V, W, Y | |
| 3 | A, E, G, I, K, Q, R, S, T, V | I, K, V |
| 4 | C | C |
| 5 | A, C, D, E, F, G, H, I, K, L, M, N, Q, | A, C, G, H, I, K, L, M, N, Q, R, |
| | R, S, T, V, W, Y | S, T, , V, W, Y |
| 6 | A, C, D, E, F, G, H, I, K, L, M, N, P, | A, C, D, E, G, H, K, L, M, N, Q, |
| | Q, R, S, T, V, W, Y | R, S, T, W, Y |
| 7 | A, C, D, E, F, G, H, I, K, L, M, N, P, | A, C, D, E, F, G, H, K, M, N, Q, |
| | Q, R, S, T, V, W, Y | R, , S, T, W, Y |
| 8 | A, C, D, E, F, G, H, I, K, L, M, N, Q, | A, C, F, H, I, K, L, M, R, T, V, |
| | R, S, T, V, W, Y | WY |
| 9 | C | C |
| 10 | A, C, E, F, H, I, K, L, M, N, Q, R, S, | C, I, V |
| | T, V, W, Y | |
| 11 | A, C, D, E, F, G, H, I, K, L, M, N, P, | A, C, D, E, H, I, K, L, M, N, Q, |
| | Q, R, S, T, V, W, Y | R, S, T, V, Y |
| 12 | A, E, G, H, M, N, Q, S, T | A, M |
| 13 | A, C, D, E, F, G, H, I, K, L, M, N, P, | A, C, D, E, H, I, K, L, M, N, p, |
| | Q, R, S, T, V, W, Y | Q, R, S, T, V, Y |
| 14 | A, C, D, E, F, G, H, K, L, M, N, P, Q, | A, C, E, F, H, K, L, M, Q, R, S, |
| | R, S, T, V, W, Y | T, W, Y |
| 15 | C | C |
| 16 | W | W |
| 17 | A, E, H, I, K, L, M, Q, V, Y | E, M, Q |
| 18 | A, C, G, S | A, C, G |
| 19 | D, F, H, N, Q, W, Y | F, W, Y |
| 20 | F, W, Y | W, Y |
| 21 | A, C, E, F, H, I, K, L, M, Q, S, T, V, | C, I, L, M |
| | W, Y | |
| 22 | A, C, F, H, I, L, M, Q, T, V, W, Y | I, L, M, W |
| 23 | A, C, D, G, H, K, M, N, Q, R, S, T, V, | A, N, S |
| | W, Y | |
| 24 | A, C, D, E, F, G, H, I, K, L, M, N, Q, | A, C, E, F, H, I, K, L, M, N, Q, |
| | R, S, T, V, W, Y | R, S, T, V, W, Y |
| 25 | A, C, D, E, F, G, H, I, K, L, M, N, P, | A, C, F, H, I, L, M, Q, V, W, Y |
| | Q, R, S, T, V, W, Y | |
| 26 | A, C, D, E, F, G, H, I, K, L, M, N, P, | A, C, E, F, G, H, I, K, L, M, N, |
| | Q, R, S, T, V, W, Y | Q, R, S, T, V, W, Y |

TABLE 9
Allowable residues per position of construct 23R_mini_17, based on the fitness of single mutants
for binding hIL-23R determined during directed evolution, without (1) or with (2) pre-treatment
with simulated intestinal fluid (SIF; see FIGS. 11C and 11D, respectively). All mutants with at
least 2-fold enrichment in the first selection relative to the naive pool are deemed allowable.

| | Allowable residues: binding | Allowable residues: binding |
|---|---|---|
| | to hIL-23R without pre- | to hIL-23R with pre- |
| Sequence | treatment with SIF | treatment with SIF |
| position | (SEQ ID NO: 86) | (SEQ ID NO: 87) |
| 1 | C, Y | C |
| 2 | A, C, D, E, F, G, H, I, K, L, M, N, Q, R, S, | A, C, E, F, G, H, I, K, L, M, Q, R, S, |
| | T, V, W, Y | T, V, W, Y |
| 3 | A, C, D, E, F, G, H, I, K, L, M, N, P, Q, R, | A, I, L, T, V |
| | S, T, V, W, Y | |
| 4 | A, C, D, E, F, G, H, I, K, L, M, N, P, Q, R, S, T, V, W, Y | A, C, E, F, H, I, K, L, M, N, Q, R, S, T, V, W, Y |
| 5 | C, V | C |
| 6 | A, D, E, G, H, K, L, M, N, P, Q, R, S, T, | G |
| | W, Y | |
| 7 | A, C, D, E, F, G, H, I, K, L, M, N, P, Q, R, S, T, V, W, Y | A, C, D, E, F, G, H, I, K, L, M, N, P, Q, R, S, T, V, Y |
| 8 | A, C, D, E, F, G, H, I, K, L, M, N, P, Q, R, S, T, V, W, Y | A, C, D, E, F, G, H, I, K, L, M, N, Q, R, S, T, V, W, Y |
| 9 | A, C, D, E, F, G, H, I, K, L, M, N, P, Q, R, | A, E, H, I, L, M, N, Q, S, T, V, Y |
| | S, T, V, W, Y | |
| 10 | A, F, I, K, L, M, R, V, W, Y | F |
| 11 | A, C, D, E, F, G, H, I, K, L, M, N, Q, R, S, T, V, W, Y | A, C, E, F, H, I, K, L, M, N, Q, R, S, T, V, W, Y |
| 12 | A, F, G, L, M, S, W, Y | A, F, G |
| 13 | A, C, D, E, F, G, H, I, K, L, M, N, P, Q, R, | A, D, E, G, H, K, N, P, Q, R, S, T, V, Y |
| | S, T, V, W, Y | |
| 14 | A, C, D, E, F, H, K, L, M, N, P, Q, R, S, | A, D, E, H, K, M, Q, S, Y |
| | V, W, Y | |
| 15 | A, G, H, I, K, L, M, N, P, Q, R, S, T, V, | H, K, L, M, N, Q, R |
| | W, Y | |
| 16 | W | W |
| 17 | E, H, I, K, M, Q | M, Q |
| 18 | C | C |
| 19 | D, E, F, H, I, M, V, W, Y | F, V, W, Y |
| 20 | F, W, Y | W, Y |
| 21 | A, F, H, I, L, Q, T, V, W, Y | F, I, V, W, Y |
| 22 | A, C, D, E, F, G, H, I, K, L, M, N, Q, R, S, | A, C, E, F, H, I, K, L, M, N, Q, R, S, T, |
| | T, V, W, Y | V, W, Y |
| 23 | A, D, E, F, G, H, K, M, N, Q, R, S, T, V, | A, H, M, N, Q, R, S, T, W, Y |
| | W, Y | |
| 24 | A, C, D, E, F, H, K, L, M, N, Q, R, S, T, | A, K, L, M, Q, R, S, Y |
| | V, W, Y | |
| 25 | A, C, D, E, F, G, H, I, K, L, M, N, Q, R, S, | C, E, F, H, M, N, Q, S, T, V, W, Y |
| | T, V, W, Y | |
| 26 | C, G, H, S, T | C |

In another embodiment, the hIL-23R binding polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:84-87.

In one embodiment of each of the above aspects, amino acid substitutions relative to the reference peptide domains are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment of any of the above aspects, the polypeptide further comprises one or more additional functional domains added at the N-terminus and/or the C-terminus of the polypeptide. Any suitable functional domain(s) may be added as suitable for an intended purpose, including but not limited to albumin (to improve serum half-life), a receptor targeting domain, molecular probes such as fluorescent proteins, a tag (including but not limited to a polyhistidine tag), etc. In one embodiment, the polypeptide further comprises one or more additional functional domains added at the C-terminus of the polypeptide.

In another embodiment of any embodiment herein, the polypeptide may further comprise a targeting domain. The targeting domain, when present may be covalently or non-covalently bound to the first polypeptide, second polypeptide, and/or polypeptide. In embodiments where the targeting domain is non-covalently bound, any suitable means for such non-covalent binding may be used, including but not limited to streptavidin-biotin linkers. In another embodiment, the targeting domain, when present, is a translational fusion with the polypeptide. In this embodiment, the polypeptide and the targeting domain may directly abut each other in the translational fusion or may be linked by a polypeptide linker suitable for an intended purpose.

The targeting domains are polypeptide domains or small molecules that bind to a target of interest. In one non-limiting embodiment, the targeting domain binds to a cell surface protein; in this embodiment, the cell may be any cell type of interest that includes a surface protein that can be bound by a suitable targeting domain. In one embodiment, the cell surface proteins are present on the surface of cells selected from the group consisting of intestinal epithelial cells, chondrocytes, or keratinocytes. In another embodiment, the targeting domain binds to a component of the extracellular matrix (ECM); in this embodiment, the ECM component may consist of collagen, elastin, or hyaluronic acid.

In a further embodiment, the polypeptides are hIL-23R antagonists. In one embodiment, the polypeptides do not detectably bind to IL-12, or bind IL-12 with very low affinity.

In a fifth aspect, the disclosure provides conditionally maximally active hIL-23R binding protein, comprising a first polypeptide component and a second polypeptide component, wherein the first polypeptide component and the second polypeptide component are not present in a fusion protein, wherein
(a) in total the first polypeptide component and the second polypeptide component comprise domains X3 and X5 as defined in any embodiment of the first aspect of the disclosure;
(b) the X3 domain is present in the first polypeptide component and the X5 domain is present in the second polypeptide component;

the first polypeptide component and the second polypeptide component are not maximally active hIL-23R binding protein individually, and wherein the first polypeptide component and the second polypeptide interact to form a maximally active hIL-23R binding protein.

As discussed herein, the X5 domain in these embodiments is sufficient for hIL-23R binding and includes the primary binding interface, while the X3 domain provides additional binding contacts that are not required for binding to hIL-23R, but expand the interaction surface permitting higher affinity and specificity in binding. The conditionally maximally active hIL-23R binding proteins of the disclosure thus provide for conditional generation of maximal hIL-23R binding activity.

All embodiments and combinations of embodiments of the first aspect of the disclosure may be used in this fifth aspect. In one embodiment, X5 comprises an alpha-helical polypeptide domain of between 12-20 amino acids in length, and wherein X5 comprises:

the amino acid sequence of residues 40-47 in SEQ ID NO:1 or 2 (see Table 1);

the amino acid sequence of residues 40-47 in the amino acid sequence selected from the group consisting SEQ ID NO: 3-6 (See Tables 2-3); or

the amino acid sequence of residues 39-54 in the amino acid sequence selected from the group consisting of SEQ ID NOS:1-6.

In another embodiment, X3 comprises a polypeptide domain between 12-20 amino acids in length, and wherein X3 comprises the amino acid sequence of residues 22-33 in the amino acid sequence selected from the group consisting of SEQ ID NOS:1-6; or the amino acid sequence of residues 21-35 in the amino acid sequence selected from the group consisting of SEQ ID NOS:1-6.

In further embodiments:
(A) X5 comprises the amino acid sequence of residues 40-47 in the amino acid sequence selected from the group consisting SEQ ID NO: 5-6 (See Table 3); and X3 comprises the amino acid sequence of residues 22-33 in the amino acid sequence selected from the group consisting SEQ ID NO: 5-6 (See Table 3); or
(B) X5 comprises the amino acid sequence of residues 39-54 in the amino acid sequence selected from the group consisting SEQ ID NO: 5-6 (See Table 3); and
(b) X3 comprises the amino acid sequence of residues 21-35 in the amino acid sequence selected from the group consisting SEQ ID NO: 5-6 (See Table 3).

In another embodiment, the first polypeptide component comprises the X1 and X2 domain of any embodiment of the first aspect of the disclosure.

In a further embodiment, X1 comprises a polypeptide domain of between 12-20 amino acids in length, and wherein X1 comprises the amino acid sequence of residues 1-16 in the amino acid sequence selected from the group consisting of SEQ ID NOS:1-6, or wherein X1 comprises the amino acid sequence of residues 1-16 in the amino acid sequence selected from the group consisting of SEQ ID NOS:5-6.

In one embodiment, X2 comprises the amino acid sequence of residues 17-20 in the amino acid sequence selected from the group consisting of SEQ ID NOS:1-6.

In another embodiment, X5, X3, and X1 when present, are each alpha helical domains. In a further embodiment of the conditionally maximally active hIL-23R binding protein:

X1, when present, comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X1 domain present in any of SEQ ID NOS: 10-74, particularly SEQ ID NO:S 69 or 74;

X2, when present, comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X2 domain present in any of SEQ ID NOS: 10-74, particularly SEQ ID NO:S 69 or 74;

X3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X3 domain present in any of SEQ ID NOS: 10-74, particularly SEQ ID NO:S 69 or 74, and X5 comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X5 domain present in any of SEQ ID NOS: 10-74, particularly SEQ ID NO: S 69 or 74.

In one embodiment, the first polypeptide component and the second polypeptide component are non-covalently associated. In another embodiment; the first polypeptide component and the second polypeptide component are indirectly bound to each other through a receptor.

In a sixth aspect, the disclosure provides conditionally maximally active hIL-23R binding protein, comprising a first polypeptide component and a second polypeptide component, wherein the first polypeptide component and the second polypeptide component are not present in a fusion protein, wherein
(a) in total the first polypeptide component and the second polypeptide component comprise domains X1 and X3 as defined in any embodiment or combination of embodiments of the second aspect of the disclosure;
(b) the X1 domain is present in the first polypeptide component and the X3 domain is present in the second polypeptide component;

the first polypeptide component and the second polypeptide component are not maximally active hIL-23R binding protein individually, and wherein the first polypeptide component and the second polypeptide non-covalently interact to form a maximally active hIL-23R binding protein.

As discussed herein, the X1 domain in these embodiments is sufficient for hIL-23R binding and includes the primary binding interface, while the X3 domain provides additional binding contacts that are not required for binding to hIL-23R, but expand the interaction surface permitting higher affinity and specificity in binding. The conditionally maximally active hIL-23R binding proteins of the disclosure thus provide for conditional generation of maximal hIL-23R binding activity.

In one embodiment, X1 comprises an alpha-helical polypeptide domain of between 12-20 amino acids in length, and wherein X1 comprises:

the amino acid sequence of residues 1-10 in the amino acid sequence selected from the group consisting of SEQ ID NOS:103-108 (See Tables 5-7); or

the amino acid sequence of residues 1-16 in the amino acid sequence selected from the group consisting of SEQ ID NOS:101-108.

In another embodiment, X3 comprises a polypeptide domain between 12-20 amino acids in length, and wherein X3 comprises:

the amino acid sequence of residues 25-33 in the amino acid sequence selected from the group consisting of SEQ ID NOS:101-108; or the amino acid sequence of residues 19-34 in the amino acid sequence selected from the group consisting of SEQ ID NOS:101-108.

In further embodiments,
(A) X1 comprises the amino acid sequence of residues 1-10 in the amino acid sequence selected from the group consisting of SEQ ID NOS:105-108 (Tables 6-7), and X3 comprises the amino acid sequence of residues 25-33 in the amino acid sequence selected from the group consisting of SEQ ID NOS:103-108; or
(B) X1 comprises the amino acid sequence of residues 1-16 in the amino acid sequence selected from the group consisting of SEQ ID NOS:105-108 (Tables 6-7); and X3 comprises the amino acid sequence of residues 19-34 in the amino acid sequence selected from the group consisting of SEQ ID NOS:103-108.

In other embodiments the first polypeptide component comprises the X4 and X5 domain of any embodiment or combination of embodiments of the second aspect of the disclosure.

In another embodiment, X5 comprises a polypeptide domain of between 12-20 amino acids in length, and wherein X5 comprises the amino acid sequence of residues 27-53 in the amino acid sequence selected from the group consisting of SEQ ID NOS:105-108, or the amino acid sequence of residues 37-53 in the amino acid sequence selected from the group consisting of SEQ ID NOS:101-108. In a further embodiment, X4 comprises the amino acid sequence of residues 35-36 in the amino acid sequence selected from the group consisting of SEQ ID NOS:101-108. In another embodiment, X1, X3, and X5 when present, are each alpha helical domains.

In various further embodiments,

X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X1 domain present in any of SEQ ID NO: 110-180, particularly SEQ ID NO: 160-163;

X3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X3 domain present in any of SEQ ID NO: 110-164 and 166-180, particularly SEQ ID NO: 160-163;

X4, when present, comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X4 domain present in any of SEQ ID NO: 110-164 and 172-180, particularly SEQ ID NO: 160-163; and

X5 comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X5 domain present in any of SEQ ID NO: 110-164 and 173-180, particularly SEQ ID NO: 160-163.

In one embodiment, the first polypeptide component and the second polypeptide component are non-covalently associated. In another embodiment, the first polypeptide component and the second polypeptide component are indirectly bound to each other through a receptor.

In a seventh aspect, the disclosure provides polypeptides comprising an X3 domain as defined herein for any embodiment of the first aspect of the disclosure, wherein the polypeptide does not include an X5 domain as defined in any embodiment of the first aspect of the disclosure.

The polypeptides of this embodiment may be used, for example, to generate the conditionally maximally active hIL-23R binding proteins of the fifth aspect of the disclosure. In various embodiments, the X3 domain comprises the amino acid sequence of residues 22-33 in the amino acid sequence selected from the group consisting of SEQ ID NOS:1-6; or the amino acid sequence of residues 21-35 in the amino acid sequence selected from the group consisting of SEQ ID NOS:1-6. In other embodiments, X3 comprises the amino acid sequence of residues 22-33 in the amino acid sequence selected from the group consisting SEQ ID NO: 5-6 (See Table 3); or wherein X3 comprises the amino acid sequence of residues 21-35 in the amino acid sequence selected from the group consisting SEQ ID NO: 5-6 (See Table 3).

In a further embodiment, the polypeptide comprises the X1 and X2 domain of any embodiment of the first aspect of the disclosure. In one embodiment, X1 comprises a polypeptide domain of between 12-20 amino acids in length, and wherein X1 comprises the amino acid sequence of residues 1-16 in the amino acid sequence selected from the group consisting of SEQ ID NOS:1-6, or wherein X1 comprises the amino acid sequence of residues 1-16 in the amino acid sequence selected from the group consisting of SEQ ID NOS:5-6. In another embodiment, X2 comprises the amino acid sequence of residues 17-20 in the amino acid sequence selected from the group consisting of SEQ ID NOS:1-6. In a further embodiment, X3 and X1 (when present) are each alpha helical domains.

In one embodiment,

X1, when present, comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X1 domain present in any of SEQ ID NOS: 10-74;

X2, when present, comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X2 domain present in any of SEQ ID NOS: 10-74; and

X3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X1 domain present in any of SEQ ID NOS: 10-74.

In an eighth aspect, the disclosure provides polypeptide comprising an X3 domain as defined herein for any embodiment of the second aspect of the disclosure, wherein the polypeptide does not include an X1 domain as defined in any embodiment of the second aspect of the disclosure. The polypeptides of this embodiment may be used, for example, to generate the conditionally maximally active hIL-23R binding proteins of the sixth of the disclosure. In one embodiment, the X3 domain is between 12-20 amino acids in length, and wherein X3 comprises:

the amino acid sequence of residues 25-33 in the amino acid sequence selected from the group consisting of SEQ ID NOS:101-108; or

the amino acid sequence of residues 19-34 in the amino acid sequence selected from the group consisting of SEQ ID NOS:101-108.

In another embodiment, X3 comprises the amino acid sequence of residues 25-33 in the amino acid sequence selected from the group consisting of SEQ ID NOS:103-108; or residues 19-34 in the amino acid sequence selected from the group consisting of SEQ ID NOS:103-108. In a further embodiment, the polypeptide comprises the X4 and X5 domain of any embodiment of the second aspect of the disclosure. In various embodiments, X5 comprises a polypeptide domain of between 12-20 amino acids in length, and wherein X5 comprises the amino acid sequence of residues 27-53 in the amino acid sequence selected from the group consisting of SEQ ID NOS:105-108, or the amino acid sequence of residues 37-53 in the amino acid sequence selected from the group consisting of SEQ ID NOS:101-108. In another embodiment, X4 comprises the amino acid sequence of residues 35-36 in the amino acid sequence selected from the group consisting of SEQ ID NOS:101-108. In a further embodiment, X3 and X5 (when present) are each alpha helical domains.

In one embodiment:

X5, when present, comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X5 domain present in any of SEQ ID NO: 110-180;

X4, when present, comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X4 domain present in any of SEQ ID NO: 110-180; and

X3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X3 domain present in any of SEQ ID NO: 110-180.

In another embodiment, the polypeptides of the seventh or eighth aspects may further comprise one or more additional functional domains added at the N-terminus and/or the C-terminus of the polypeptide. Any suitable functional domain(s) may be added as suitable for an intended purpose, including but not limited to albumin (to improve serum half-life), a targeting domain, a receptor targeting domain, a molecular probe such as a fluorescent protein, a polypeptide sequence to aid in detection or purification (including but not limited to a polyhistidine tag), an N-terminal polypeptide sequence to enable secreted or enhanced expression in various organisms (including but not limited to Escherichia coli, Bacillus subtilis, Saccharomyces cerevisiae, Kluyveromyces lactis, spirulina, or mammalian systems), etc. In one embodiment, the polypeptide further comprises one or more additional functional domains added at the C-terminus of the polypeptide.

In a further embodiment, the first polypeptides, second polypeptides, and polypeptides of any embodiment or aspect herein may further comprise a targeting domain. In this embodiment, polypeptides can be directed to a target of interest. The targeting domain may be covalently or non-covalently bound to the first polypeptide, second polypeptide, and/or polypeptide. In embodiments where the targeting domain is non-covalently bound, any suitable means for such non-covalent binding may be used, including but not limited to streptavidin-biotin linkers.

In another embodiment, the targeting domain, when present, is a translational fusion with the polypeptide, first polypeptide, and/or second polypeptide. In this embodiment, the polypeptide and the targeting domain may directly abut each other in the translational fusion or may be linked by a polypeptide linker suitable for an intended purpose.

The targeting domains are polypeptide domains or small molecules that bind to a target of interest. In one non-limiting embodiment, the targeting domain binds to a cell surface protein; in this embodiment, the cell may be any cell type of interest that includes a surface protein that can be bound by a suitable targeting domain. In one embodiment, the cell surface proteins are present on the surface of cells selected from the group consisting of intestinal epithelial cells, chondrocytes, or keratinocytes.

In another embodiment, the targeting domain binds to a component of the extracellular matrix (ECM); in this embodiment, the ECM component may consist of collagen, elastin, or hyaluronic acid.

In all embodiments herein, the targeting domains can be any suitable polypeptides that bind to targets of interest and can be incorporated into a polypeptide of the disclosure. In non-limiting embodiments, the targeting domain may include but is not limited to an scFv, a F(ab), a F(ab′)2, a B cell receptor (BCR), a DARPin, an affibody, a monobody, a nanobody, diabody, an antibody (including a monospecific or bispecific antibody); a cell-targeting oligopeptide including but not limited to RGD integrin-binding peptides, de novo designed binders, aptamers, a bicycle peptide, conotoxins, small molecules such as folic acid, and a virus that binds to the cell surface.

In one embodiment of the conditionally maximally active hIL-23R binding protein of any embodiment of the fifth and sixth aspects herein, the first polypeptide component further comprises a first targeting domain and/or the second polypeptide component further comprises a second targeting domain. The first targeting domain and the second targeting domain may be the same or may be different, as deemed appropriate for an intended use.

In one embodiment, the first polypeptide component further comprises a first targeting domain and the second polypeptide component further comprises a second targeting domain. In another embodiment, the first targeting domain, when present, is a translational fusion with the first polypeptide, and the second targeting domain, when present, is a translational fusion with the second polypeptide. In one embodiment, the first targeting domain and/or the second targeting domain each bind to cell surface proteins.

In one embodiment, the hIL-23R binding polypeptide or conditionally maximally active hIL-23R binding protein of any of aspect, embodiment, or combination of embodiments disclosed herein, binds to hIL-23R with a binding affinity of 50 nm, 25 nm, 10 nm, 5 nm, 1 nm, 0.75 nm, 0.5 nm, 0.25 nm, 0.1 nm, or less as measured by biolayer interferometry surface plasmon resonance. In one embodiment, the measurement conditions are as detailed in the examples that follow.

In a ninth aspect, the disclosure provide multimers comprising two or more copies of the hIL-23R binding polypeptide, conditionally maximally active hIL-23R binding protein, polypeptide, or polypeptide component of any of embodiment or combination of embodiments disclosed herein. The multimers of the disclosure comprise 2, 3, 4, 5, 6, 7, 8, 9, 10, or more copies of the recited component. In some embodiments, the multimer may comprise a translational fusion of two more copies of the same recited component, which may be separated by optional amino acid linkers, such as generic flexible linkers. In other embodiments, the multimer may comprise a translational fusion of two more different recited components. In other embodiments, the two or more recited components may be present on a scaffold that presents the recited components on its surface. Any suitable scaffold may be used, including but not limited to natural or synthetic multimerizing polypeptide scaffolds with two or more interacting subunits including virus-like particles or synthetic nanocages, synthetic polymers including polyethylene glycol (PEG), beads, etc.

In a further aspect, the present disclosure provides nucleic acids, including isolated nucleic acids, encoding the polypeptides and polypeptide components of the present disclosure that can be genetically encoded. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present disclosure provides expression vectors comprising the nucleic acid of any aspect of the invention operatively linked to a suitable control sequence. “Expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector. In some embodiments, the expression vector can be administered in the methods of the disclosure to express the polypeptides in vivo for therapeutic benefit. In non-limiting embodiments, the expression vectors can be used to transfect or transduce cell therapeutic targets (including but not limited to CAR-T cells or tumor cells) to effect the therapeutic methods disclosed herein.

In a further aspect, the present disclosure provides host cells that comprise the expression vectors, polypeptides, polypeptide components, conditionally maximally active hIL-23R binding proteins, multimers, and/or nucleic acids disclosed herein, wherein the host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (R. I. Freshney. 1987. Liss, Inc. New York, NY)). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

In another aspect, the present disclosure provides pharmaceutical compositions, comprising the polypeptide, polypeptide component, conditionally maximally active hIL-23R binding protein, nucleic acid, expression vector, or cell of any embodiment or combination of embodiments herein and a pharmaceutically acceptable carrier. The pharmaceutical compositions of the disclosure can be used, for example, in the methods of the disclosure described herein. The pharmaceutical composition may further comprise (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptide, polypeptide component, conditionally maximally active hIL-23R binding protein, nucleic acid, expression vector, or cell of any embodiment or combination of embodiments herein may be the sole active agent in the pharmaceutical composition, or the composition may further comprise one or more other active agents suitable for an intended use.

In a further aspect, the disclosure provides methods for treating a disorder selected from the group consisting of inflammatory bowel disease (IBD) (including but not limited to includes Crohn's disease and ulcerative colitis), psoriasis, atopic dermatitis, rheumatoid arthritis, psoriatic arthritis, osteoarthritis, axial and peripheral spondyloarthritis, ankylosing spondylitis, enthesitis, and tendonitis, comprising administering to a subject in need thereof an amount effective to treat the disorder of the polypeptide, polypeptide component, conditionally maximally active hIL-23R binding protein, nucleic acid, expression vector, cell, or pharmaceutical composition of any embodiment or combination of embodiments herein. As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The subject may be any subject that has a relevant disorder. In one embodiment, the subject is a mammal, including but not limited to humans, dogs, cats, horses, cattle, etc.

### EXAMPLES

We have developed computationally designed, hyper-stable peptides targeting the IL-23 receptor (IL-23R) that represent a new oral, gut-restricted mode of treatment for IBD.

Here, to design IL-23R antagonists, we incorporated a native hotspot from IL-23 cytokine and additional computationally determined hotspots into highly stable, de novo designed miniprotein scaffolds. We used directed evolution by yeast surface display (YSD) to further enhance affinity and proteolytic stability in conditions mimicking intestinal fluid. Inhibitors with highest stability and affinity for IL-23R were tested in vitro to confirm inhibition of IL-23-mediated cell signaling.

### Results

Computational design yields low nanomolar inhibitors of IL-23R

IL-23 is a heterodimeric cytokine composed of the p19 subunit unique to IL-23 and the p40 subunit shared with IL-12. The IL-23 receptor is likewise heterodimeric including a unique subunit, IL-23R, and a shared subunit, IL-12RB1. While IL-12 and IL-23 share cytokine and receptor subunits, they have unique roles in inflammation and immunity. IL-12 promotes differentiation of Th1 cells and stimulates production of IFNg, while IL-23 promotes differentiation and maintenance of Th17 cells and stimulates production of IL-17. Recent studies have determined that IL-23 and not IL-12 drives pathogenic autoinflammation, and antibodies targeting the IL-23 unique p19 subunit have indeed shown better efficacy and safety in treating autoinflammatory diseases than STELARA®, which targets the IL-12/23 shared p40 subunit.

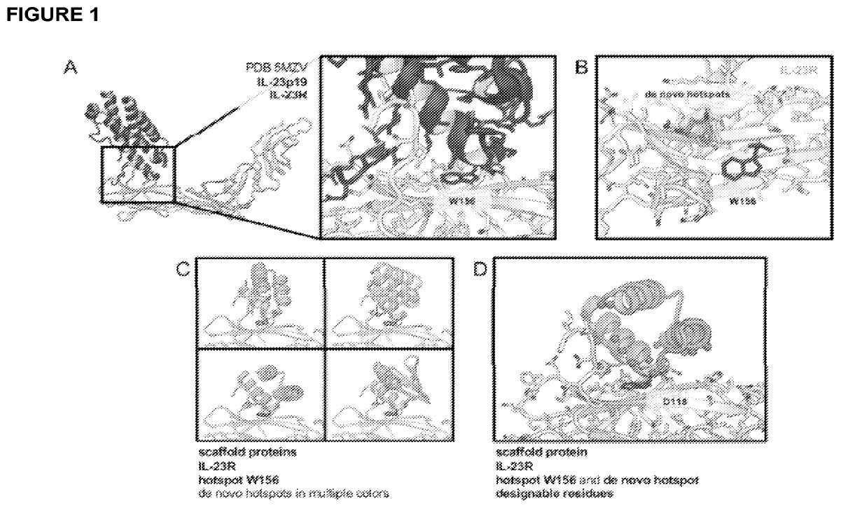

The crystal structure of IL-23 heterodimer in complex with IL-23R (PDB 5MZV) shows site III of the 4-helix bundle p19 subunit interacting with a hydrophobic surface of IL-23R. As a first step in designing IL-23R inhibitors that compete with p19, we selected p19 residue W156 as a hotspot to seed design (FIG. 1A). As typical protein-protein interactions have a buried surface area of >1,000 A², we computationally generated a rotamer interaction field (RIF), i.e. disembodied residues that favorably interact with IL-23R surface residues, to supplement the native hotspot and expand the interaction surface (FIG. 1B). Next, thousands of de novo designed miniproteins with diverse topologies and experimentally validated stability were docked at the IL-23R surface such that the native Trp hotspot and additional de novo hotspots were incorporated (FIG. 1C). Then, with each docked configuration as input, the Rosetta™ molecular modeling suite was used to mutate scaffold residues at the IL-23R interface to side chains favoring high-affinity binding (FIG. 1D). Native and de novo hotspots, scaffold residues in the scaffold hydrophobic core, and scaffold residues far from the IL-23R interface were not allowed to mutate. The resulting designed inhibitor candidates were filtered on computational metrics thought to predict high binding affinity and inhibitor monomer stability, and genes encoding the best 15,000 were commercially synthesized and transformed into yeast for surface display. Yeast were selected for binding to labeled recombinant human IL-23R (hIL-23R) by multiple successive rounds of fluorescence-activated cell sorting (FACS). Naive and sorted pools were analyzed by next-generation sequencing (NGS) and designs were ranked by their relative enrichment or depletion. The most enriched design sequences can be found in Table 10.

TABLE 10
Enriched computational design sequences

>23R_A
MESEKYLRELVKKYYEGKLSVQEAVEEVRKYARKKGLEAWMLTWMEMELVKRYI (SEQ ID NO: 75)
>23R_B
NLWQIFYQLSTILKRTGDPTAKKLLKALAEALKKGDEKALKELAKKATKYIRS (SEQ ID NO: 76)
>23R_C
PEELRRRVEHFLRQGVERWMVMWMLIQQGFDNKEVWKVLQEVL (SEQ ID NO: 77)
>23R_D
ELWFLMALVIAACLWAWKASNVEEAEEALWWALVMAREANMROLEEFVKRMREEVRRHL (SEQ ID NO: 78)
>23R_E
DVVVLLGLVIVIPERWRLWLIVVIAARVMGLRVEVHGHVIIVI (SEQ ID NO: 79)
>23R_F
TVVIINGVPFWFEFLWELFIFALLMALALGLKIEFHGELIEVK (SEQ ID NO: 80)
>23R_G
TPARELVRELVALALLIAVLRNDIVRLTLDVQGFKITVDVDAHWEAFVRILLLAEILVLEWLKG
(SEQ ID NO: 81)
>23R_H
DWRELMLVWMALWWIWWAFLAGVPLIVEVVVNGLHFRVIVDRDPTTNKRALDIMLWVWEWLATL
(SEQ ID NO: 82)
>23R_I
GRWELLVLAYLALLLGAAEAVWRLLELAKKLGDEMAFRWILELWERAL (SEQ ID NO: 83)

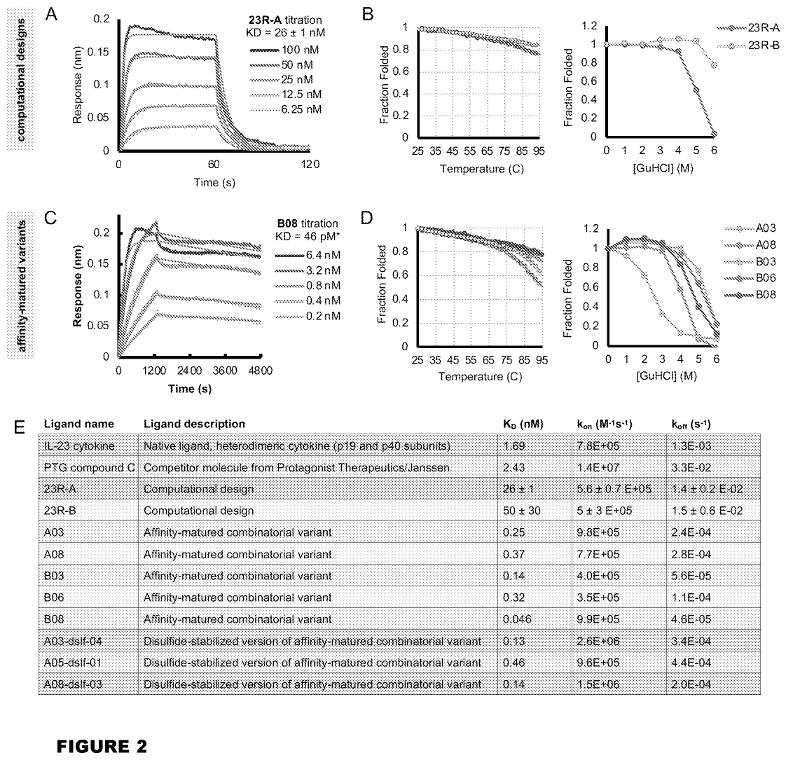

Two designs, 23R_A (SEQ ID NO:10) and 23R_B (SEQ ID NO:110), were highly enriched in the final selection pool and were chosen for further biochemical characterization. Both designs are 3-helix bundles (54 and 53 residues, respectively) which incorporate the native Trp hotspot at the N-terminal end of a helix such that IL-23R residue D118 forms a helix-capping hydrogen bond with the Trp hotspot's free backbone amine, similar to the αD helix of p19 (FIG. 1D). In both designs, this central binding helix has a larger interaction surface with hIL-23R, mediated by additional de novo hotspots, than p19. The two additional helices stabilize the central binding helix and make additional contacts with hIL-23R. Designs were expressed in E. coli and purified by immobilized metal affinity chromatography (via 6-histidine tag) and further by size exclusion chromatography. Biolayer interferometry binding titrations showed that 23R_A and 23R_B bind hIL-23R with 26±1 nM and 70±50 nM affinity, respectively (FIG. 2A,E). Both designs demonstrated high stability by circular dichroism (CD) at high temperature and high concentrations of chemical denaturant (FIG. 2B).

### An Initial Round of In Vitro Evolution Enhances Affinity 1000-Fold to Low pM

While we successfully achieved low nanomolar affinity using only computational design, higher affinity will improve competion with IL-23 cytokine which binds the receptor at 1.7 nM. To evolve 23R_A and 23R_B for higher affinity for hIL-23R, we performed deep mutational scanning. Site saturation mutagenesis (SSM) libraries, representing each possible single-position mutation based on the original designs, were transformed into yeast for surface display, and we performed two rounds of selection for binding hIL-23R by FACS. NGS analysis of SSM naive and sorted pools allowed us to calculate the fitness of each mutant for binding. The sequence fitness landscapes can be found in FIG. 8A (23R_A) and 8B (23R_B). The sequence fitness landscapes confirm the designed binding mode, as positions interacting with IL-23R in the design model have higher entropy than non-interacting positions, and the native Trp hotspot (W40 in 23R_A, W3 in 23R_B) is highly conserved.

To further enhance affinity, we incorporated mutations previously shown to enhance binding in a combinatorial library that was then sorted for binding hIL-23R to convergence in 6 rounds. The final selection pools were plated on solid media, and individual clones sequenced. 26 unique combinatorial variants appearing in the final selection pool (SEQ ID NOS: 11-24 and 111-122) were selected for expression in E. coli and further biophysical characterization.

Variants were first screened for relative binding to hIL-23R with biolayer interferometry (BLI). hIL-23R was immobilized on the BLI sensor tips and binding to each variant in solution at a constant concentration (50 nM) was measured to qualitatively determine relative performance of the variants. For the best-performing variants, binding constants (K_{D}, including kₒₙ, and k_{off}) were quantitatively determined with BLI titration experiments in triplicate. The best combinatorial variants bound IL-23R with 50-400 pM affinity, approximately a 500-fold improvement from the computational designs (FIG. 2C), and maintained high resistance to heat and chemical denaturant (FIG. 2D).

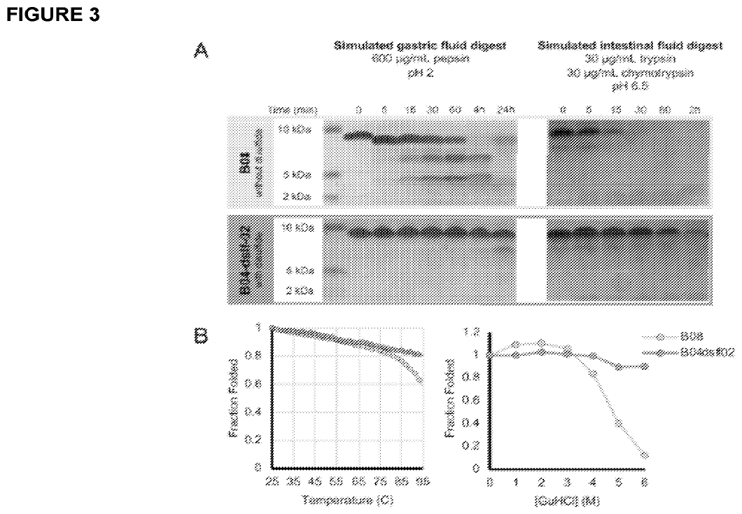

While resistance to high heat is important for manufacturing and storage, and resistance to chemical denaturant is a good proxy for stability overall, any oral, gut-restricted IL-23R inhibitor will preferably survive the harsh conditions of the gastrointestinal tract, including high acidity and physiological proteases, to reach the site of action intact. To determine feasibility of oral administration, we therefore more directly assessed the stability of our designed IL-23R inhibitors in simulated gastric fluid (SGF), including the protease pepsin at pH 2, and simulated intestinal fluid (SIF), including proteases trypsin and chymotrypsin at pH 6.5. Proteolysis was assessed qualitatively by SDS PAGE at timepoints up to 24 hours. The highest affinity combinatorial variants survive SGF with a t_{1/2} of approximately 45 minutes and SIF with t_{1/2} less than 15 minutes (FIG. 3).

### Computationally Designed Disulfide Crosslinks and In Vitro Evolution Enhance Proteolytic and Thermal Stability

In order to improve proteolytic stability, we computationally designed inhibitor variants cross-linked with intramolecular disulfide(s) (SEQ ID NOS: 25-32 and 123-129). All combinatorial variants sequenced from the final pool were modeled with up to two disulfides and filtered by disulfide geometry. The best designs were expressed in E. coli, screened for binding by BLI and for stability by SGF and SIF digest and CD. Disulfide-crosslinked variants largely retained high affinity for hIL-23R, with Kos from 130 to 460 pM (FIG. 2E), but saw significant improvement in stability with the most stable having SGF t_{1/2} of >24 hours, SIF tin of about 1 hour, as well as improved resistance to thermal and chemical denaturation (FIG. 3).

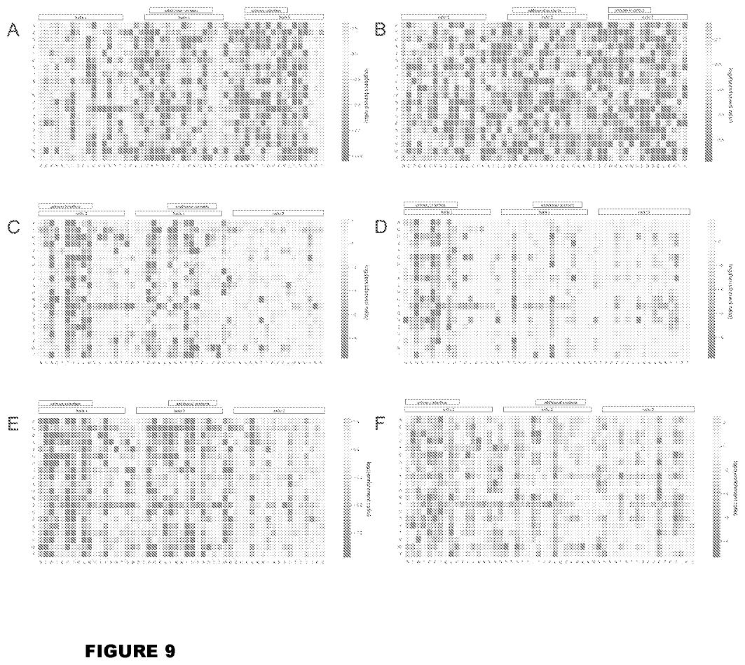

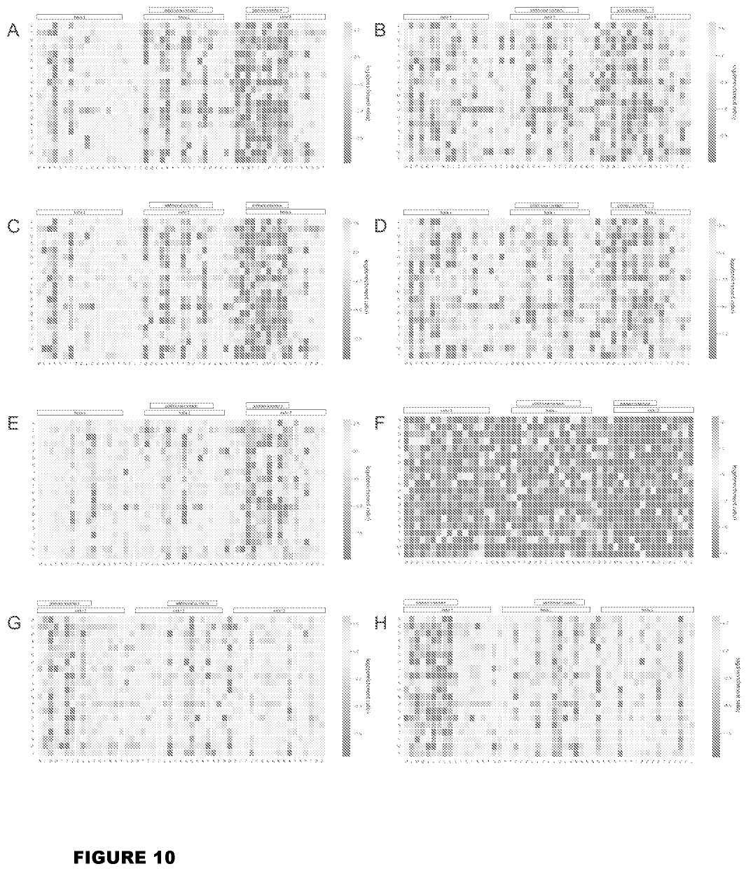

To further optimize the inhibitor sequences for stability in SIF, we carried out in vitro evolution using YSD. SSM libraries were generated based on the most stable disulfide-crosslinked variants. Yeast libraries were first incubated in SIF at 30C, then washed thoroughly and incubated with labeled hIL-23R, and cells retaining the highest binding signal (top 1-5%) were collected by FACS. Two rounds of selection were performed for each library, and the SIF incubation time and/or concentration of proteases were increased from first to second round. Unlike previous studies, we did not sort on inhibitor expression assessed via a C-terminal Myc tag, because it is possible the Myc tag can be cleaved and leave a binding-competent inhibitor on the yeast surface. Indeed in several libraries we saw a large population of Myc-negative, binding-positive cells. In parallel, we performed two rounds of selection for binding to hIL-23R only, without pre-incubation in SIF. From NGS analysis we identified mutations that enhanced both affinity and stability (FIGS. 9 and 10) and a few selected mutants were expressed in E. coli and characterized (SEQ ID NOS: 63-69 and 130-134). NGS analysis also demonstrated that BO4dslf02 can be truncated at many different locations past the first helix and maintain the ability to bind hIL-23R (SEQ ID NOS: 165-180). Best mutations were included in combinatorial libraries, sorted as above for 5-7 rounds after which individual clones were sequenced. Variants present in the final sorts were expressed in E. coli and characterized (SEQ ID NOS: 70-74 and 153-164).

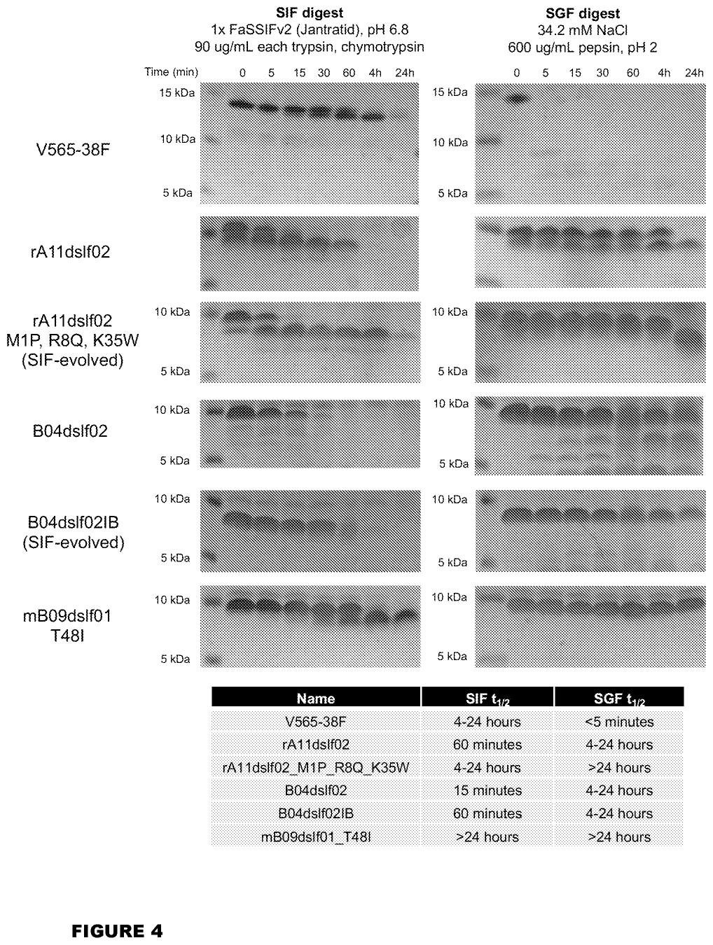

Directed evolution significantly improved SIF resistance of parent designs rA11dslf02 and B04dslf02 (FIG. 4). Several reported formulations of simulated intestinal fluid show variable proteolytic activity, and protease activity of human intestinal fluid has not been thoroughly studied. Therefore, as a benchmark for SIF stability, we directly compared our designs to V565-38F, an oral, gut-restricted nanobody inhibitor of TNFa currently in a phase 2 clinical trial for Crohn's disease, in SIF with enzyme concentrations sufficient to degrade this molecule over 24 hours. Design variants with proteolytic stability greater than or equal to V565-38F are likely to be sufficiently stable to enable oral therapy. Mutations selected to enhance SIF stability indeed improved the SIF t_{1/2} of rA11dslf02 from 60 minutes to 4-24 hours (variant rA11dslf02_M1P_R8Q_K35W [SEQ ID NO: 69]) and the SIF t_{1/2} of B04dslf02 from 5-15 minutes to 30-60 minutes (variant B04dslf02IB [SEQ ID NO: 161]). In comparison to oral nanobody V565-38F, rA11dslf02_M1P_R8Q_K35W was similarly stable in SIF and much more stable in SGF compared; this result is consistent with the reported SGF and SIF stabilities of V565-38F. Both evolved variants maintained high resistance to SGF with t_{1/2} 4-24 hours. Binding affinity to hIL-23R was measured with surface plasmon resonance (SPR); K_{D} of rA11dslf02 M1P_R8Q_K35W was 75 pM and K_{D} of BO4dslf02IB was <1 pM (dissociation rate too slow to be accurately measured by the instrument).

In parallel, we generated inhibitors with enhanced binding affinity for rat and mouse IL-23R (rIL-23R, mIL-23R). While the best human IL-23R inhibitors bind both human and rat IL-23R in vitro with similar affinity, they show negligible binding to the mouse homolog. This is consistent with PTG Compound C, which likewise binds human and rat but not mouse IL-23R (mIL-23R). As a proof of concept that an orally administered IL-23R inhibitor can treat colitis, we plan to compare designed inhibitors to relevant controls in both rat and mouse models of colitis. Unfortunately, only chemically induced models of colitis (TNBS, DSS) are readily available in rats; these models tend to show high variability within and between experiments. Generating an inhibitor that potently blocks mouse IL-23R enables access to more consistent and physiologically relevant disease models with demonstrated dependence on IL-23, such as autoreactive T-cell transfer and Mdr1a KO models, which are readily available only in mice and not rats. Thus, to generate the most potent molecules for experiments in rat and mouse, we screened existing hIL-23R-targeting libraries for variants with enhanced binding to rIL-23R and mIL-23R. The best combinatorial variants (SEQ ID NOS: 33-46, 135-149) were computationally modified to incorporate intracellular disulfide bond(s) (SEQ ID NOS: 47-62, 150-152). Hits were further optimized by directed evolution for stability and affinity to rIL-23R, hIL-23R, or mIL-23R as described above (FIG. 10). The best mIL-23R inhibitor after optimization for stability and mIL-23R affinity, mB09dslf01-T48I, is stable in both SGF and 3x SIF with t_{1/2} values greater than 24 hours (FIG. 4).

### C-Terminal Affinity Tag Enhances Proteolytic Stability of B04dslf02IB

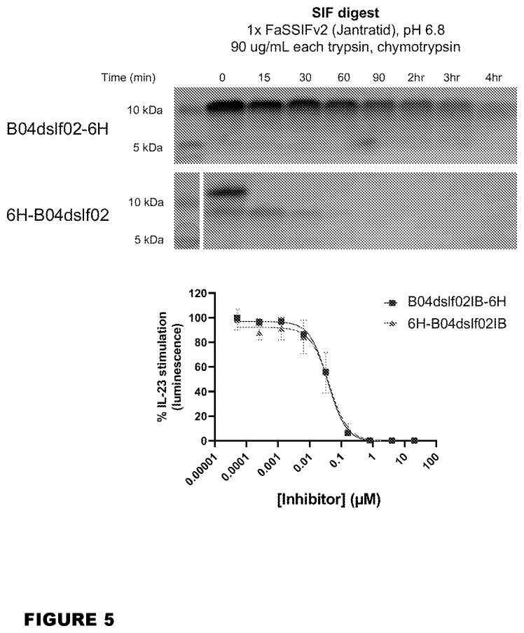

Low cost of goods is critical for an orally administered therapy treating a chronic disease. Therefore we have tested various gene and protein sequences for improved expression titer in E. coli, including various peptide tags at either or both N- and C-termini of the designed IL-23R inhibitors. Addition of a C-terminal 6-histidine tag, but not an N-terminal 6-histidine tag, greatly enhances the proteolytic stability of B04dslf02IB, but has no effect on potency (FIG. 5).

### 53-Residue Inhibitors can be Computationally Minimized to Enhance Tissue Penetrance

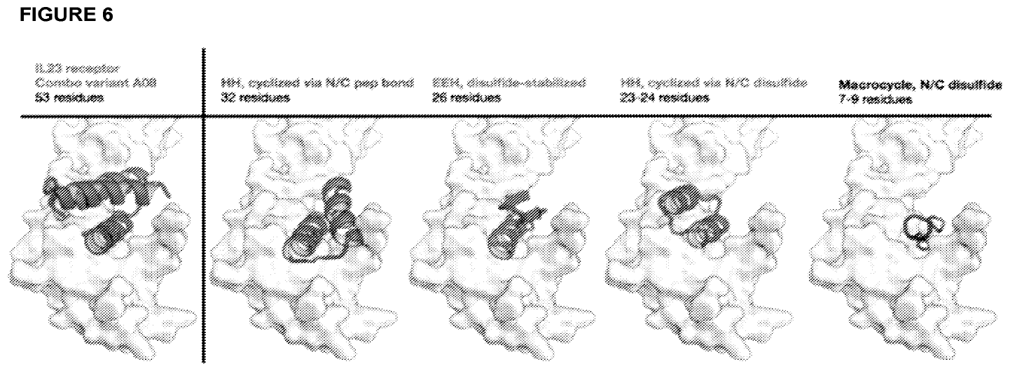

Multiple biophysical characteristics impact intestinal permeability, molecular weight among them. We may achieve better tissue penetrance in inflamed and perhaps even healthy intestinal tissue if we further reduce the size of the inhibitor. Toward this goal, we computationally designed hIL-23R inhibitors with 7 to 32 residues corresponding to molecular weights of 0.8 to 4 kDa. Starting from models of the 53-residue, high-affinity combinatorial variants as guides, we used several approaches for design (FIG. 6): (i) we isolated 6- to 14-residue motifs from the primary binding helix that included the native Trp and at least one de novo hotspot, then used the MotifGraft™ protocol to place the motif at any accommodating positions on structurally validated 26- to 32-residue scaffolds, (ii) starting from an 11-residue helical motif similar to that in (i), we built a second de novo helix antiparallel to the motif, with the two helices cross-linked by a disulfide between N- and C-terminal cysteines, and (iii) we isolated the native Trp hotspot and a de novo hotspot conserved during directed evolution, additionally generated new de novo hotspots, and then docked computationally generated 7- to 13-residue peptides crosslinked by a disulfide between N- and C-terminal cysteines. Binding interfaces were designed and inhibitor candidates filtered as described previously, and genes for the best candidates were synthesized and transformed into yeast for screening for binding hIL-23R by FACS. The designs most enriched in the final FACS sorts are listed in SEQ ID NOS: 181-198. Based on the designs most enriched in the final FACS sorts (23R_mini_14 and 23R_mini_17), SSM libraries were generated and screened for stability and affinity by sequential incubation in SIF and labeled hIL-23R as described above (FIG. 11). Combinatorial libraries were generated as above and likewise sorted. Combinatorial variants based on the best 23R_mini_14 and 23R_mini_17 most enriched for stability and affinity to hIL-23R are listed as SEQ ID NOS: 199-228. Allowable residues per position of construct 23R_mini_14 and 23R_mini_17, based on the fitness of single mutants for binding hIL-23R were determined during directed evolution, without (1) or with (2) pre-treatment with simulated intestinal fluid (SIF; see FIGS. 11A and 11B, respectively). All mutants with at least 2-fold enrichment in the first selection relative to the naive pool are deemed allowable, and are provided in Tables 8 and 9, respectively.

### Designed Inhibitors Block IL-23-Mediated Cell Signaling In Vitro

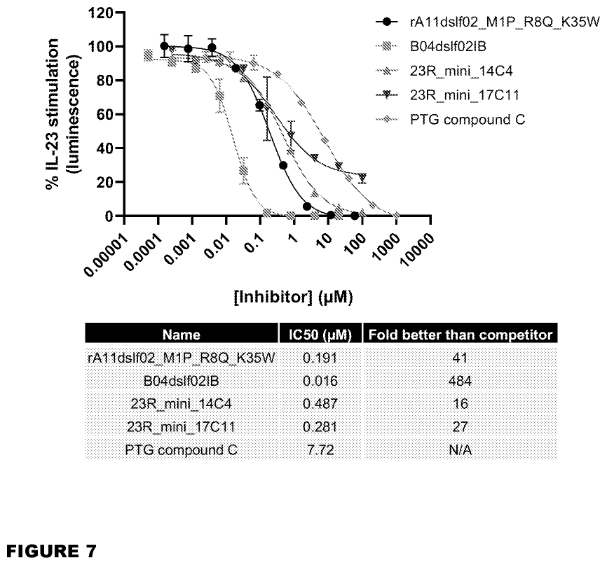

Next, we assessed the ability of IL-23R inhibitors to block IL-23-mediated cell signaling. Reporter cells expressing IL-23R linked to downstream expression of luciferase were pretreated with inhibitor or control, then stimulated with a constant concentration of human IL-23 cytokine. IC50 values were determined using linear regression to fit dose response. Our inhibitors were directly compared to PTG Compound C in this assay and demonstrated potencies 16- to 480-times greater (FIG. 7).

### DISCUSSION

Here we report the de novo design and in vitro optimization of an ultrapotent inhibitor of IL-23R as an oral, gut-restricted therapy for IBD. Our inhibitors binds hIL-23R with picomolar affinity, resulting in potent inhibition of IL-23-mediated cell signaling superior to PTG Compound C. Our inhibitors are resistant to high heat, chemical denaturant, acid, and physiological proteases, suggesting that intact transit to the inflamed gut after oral administration can be achieved with standard drug formulations.

### Materials and Methods

Computational Design of Inhibitors Targeting hIL-23R

We used the crystal structure of human IL-23R in complex with IL-23p19 and IL-23p40 (PDB 5MZV) as a starting point for design. We aimed to bind IL-23R, the IL-23-specific receptor subunit, and inhibit its interaction with IL-23p19, the IL-23-specific cytokine subunit. From the crystal structure, we first isolated IL-23R and p19 native hotspots L56, W156, L160, and L161. To supplement the native hotspots, a rotamer interaction field (RIF) of de novo hotspots was generated around selected IL-23R residues near the surface of interest, including:

G24, 125, T26, N27, 128, N29, C30, S31, G32, H33, 134, V36, T40, 150, A54, A55, 156, K57, N58, C59, Q60, P61, K63, L64, H65, F66, Y67, K68, N69, G70, 171, K72, P95, H96, A97, S98, M99, Y100, C101, T102, A103, E104, C105, P106, K107, H108, F109, Q110, E111, T112, L113,1114, C115, G116, K117, D118,1119, S120

The RIF residues (disembodied amino acid side chains) are generated such that the side chain atoms form favorable polar and apolar interactions with the given IL-23R surface residues.

In parallel, 12,345 scaffold proteins (inert de novo designed proteins with experimentally validated stability) were roughly placed at the desired IL-23R interaction surface using PatchDock. After RIF generation and initial scaffold placement, scaffolds were docked with higher resolution at the IL-23R interaction surface such that the backbone atoms of the native hotspot (in order of preference: W156, L161, L56, L160) and de novo hotspots were matched with appropriate backbone atoms of each scaffold protein, replacing the amino acid previously at that scaffold position. All other scaffold residues, previously computationally optimized for the lowest monomer free energy, were retained. This step generated 130,343 docked configurations.

Each docked configuration was input into a Rosetta™ design protocol to optimize additional scaffold residues at the IL-23R interface for high-affinity binding. Only scaffold side chains within 8 Å of the IL-23R surface were allowed to mutate. Scaffold sidechains at surface positions further than 8 Å were not allowed to mutate, but were allowed to optimize rotamer conformation. IL-23R residues within 8 Å of the scaffold were allowed to optimize rotamer conformation. All IL-23R and scaffold backbone atoms, all scaffold monomer core side chains, and IL-23R side chains further than 8 Å from the scaffold were not allowed to move.

Designed IL-23R:inhibitor complexes were filtered on metrics thought to predict high-affinity binding, including but not limited to inhibitor monomer free energy, binding energy, shape complementary of the inhibitor to the IL-23R surface, buried apolar surface area at the interface, and buried unsatisfied polar atoms. Designs with the best metrics were selected for experimental testing.

### Yeast Library Preparation, Selection and Analysis

### DNA Preparation

DNA encoding the initial design library was commercially synthesized (Agilent). For site saturation mutagenesis (SSM) libraries, in some instances full-length genes were commercially synthesized (Agilent), and in other instances libraries were prepared using overlap PCR with custom primers (Integrated DNA Technologies) as described previously.²⁵ Combinatorial libraries were prepared by gene assembly from custom oligos; oligos were designed such that all included mutations were represented either individually or as degenerate codons encoding two or more desired mutations. Oligo overlap regions had a minimum length of 12 bp and minimum melt temperature of 40° C., enabling efficient gene assembly.

All yeast libraries, including the initial design library, SSM libraries, and combinatorial libraries, were prepared with overhangs>20 bp to enable homologous recombination with the plasmid backbone (pETCON) for yeast expression and surface display via fusion to Aga2p.²⁶ For initial SSM and combinatorial libraries for affinity-maturation, the reported pETCON3 vector was used. For SSM and combinatorial libraries built with the objective of enhancing stability in simulated intestinal fluid (SIF), a pETCON variant optimized for enhanced proteolytic stability of Aga2p and Myc-tag was used.

### Fluorescence-Activated Cell Sorting (FACS)

Yeast strain EBY100 was transformed with each library and vector by electroporation and grow in minimal media selective for the yeast strain (-ura) and the transforming plasmid (-trp).²⁷ Expression was induced with 2% galactose. Surface expression was detected with anti-Myc-FITC (Immunology Consultants Laboratory) conjugate, and binding to biotinylated IL-23R was detected with streptavidin-PE (Invitrogen).

The initial design library, and SSM and combinatorial libraries meant for affinity-maturation only (before stability enhancement) were prepared for selection as follows: after 16-24 hours induction, yeast were spun down, washed with PBS with 1% FBS (PBSF), and incubated for 30-60 minutes with biotinylated target at the given concentration. Yeast were then washed with PBSF and incubated for 2-5 minutes with stain solution (1:100 each anti-Myc-FITC and streptavidin-PE), washed, and resuspended for analysis and selection by FACS. FACS consecutive gates were set as follows: (1) cell granularity and size, selecting for yeast cells (BSC vs. FSC); (2) cell morphology, selecting singlets (FSC-height vs. FSC-width); (3) expression, selecting expressors by proxy of the Myc-tag (FITC fluorescence histogram); and (4) binding signal, selecting the top 1-5% relative to total population (PE vs. FITC).

SIF SSM and combinatorial libraries were prepared as follows: after 16-24 hours induction, yeast were spun down, washed with PBSF, resuspended in SIF (recipe described below) at an OD of 2.0, and incubated at 30° C. shaking for 30-90 minutes as noted. After SIF digest, cells were spun down and washed 4 times with 800 uL PBSF, manually aspirating the supernatant each time to ensure complete washing to remove proteases. SIF-treated cells were then treated with target protein as described above. FACS gates were set similarly, but gate 3 (expressors) was excluded, as the vast majority of pools showed populations of Myc-negative, binding(PE)-positive cells, indicating that the Myc-tag was cleaved leaving binding-competent design variants displayed on the cell surface.

Generally, design and combinatorial libraries were sorted to convergence in 4-6 consecutive rounds, and SSM libraries were sorted in two consecutive rounds and deep sequenced. The concentration of target protein (human, rat, or mouse IL-23R) was decreased as sorting rounds progressed in order to efficiently separate the highest-affinity variants. In the case of SIF SSM and combinatorial libraries, protease concentrations in SIF as well as the digest duration were increased with consecutive rounds, in addition to decreasing concentration of target.

### Deep Mutational Scanning

From SSM naive and sorted pools, DNA was prepared and sequenced as follows: Yeast were lysed with 125 U/ml Zymolase at 37° C. for 5 hr, and DNA was harvested (Zymoprep™ kit from Zymo Research). Genomic DNA was digested with 2 U/μl Exonuclease I and 0.25 U/μl. Lambda exonuclease (New England Biolabs) for 90 min at 30° C., and plasmid DNA purified with a QIAquick™ kit (Qiagen). DNA was deep sequenced with a MiSeq™ sequencer (Illumina): genes were PCR amplified using primers that annealed to external regions within the plasmid, followed by a second round of PCR to add flanking sequences for annealing to the Illumina flow cell oligonucleotides and a 6 bp sample identification sequence, or barcode. PCR rounds were 12 cycles each with high-fidelity Phusion™ polymerase. Barcodes were read on a MiSeg™ sequencer using either a 300-cycle or 600cycle reagent kit (Illumina), and sequences were analyzed with adapted scripts from Enrich (Fowler et al., 2011).

### Protein Expression and Purification

All designed proteins and V565-38F were expressed cloned into the pET29b plasmid for expression from the T7 promoter, between NdeI and XhoI cut sites, incorporating a C-terminal 6-histidine tag for downstream affinity chromatography. E. coli were transformed with the resulting plasmids: strain BIL21*(DE3) (Invitrogen) for initial computational designs and affinity-matured combinatorial variants or strain Shuffle T7 (New England Biolabs) for all constructs containing disulfides. E. coli were grown to OD600 in Terrific Broth II media (MP Biomedicals) at 37° C. (BL21) or 30° C. (Shuffle T7), then expression was induced with IPTG added to 0.5 mM overnight at growth temperature or 18° C. Cells were harvested, lysed by sonication, and lysate cleared by centrifugation. Cleared lysate was incubated with NiNTA resin for 30 minutes rocking to allow binding of recombinant protein via the 6-histidine tag, then applied to a gravity column (Biorad), washed and elute& concentrated and further purified by gel filtration chromatography (AKTA Pure, Cytiva; Superdex™ 75 increase and Superdex™ 5200 increase columns, GE Life Sciences).

A custom human IL-23R construct with C-terminal avi and his tags (for enzymatic biotinylation and affinity chromatography, respectively) was commercially produced, expressed from a stable insect cell line, hIL-23R was enzymatically biotinylated via the avi-tag using recombinant BirA enzyme (Avidity). A similar rat IL-23R construct was produced, by transient expression in Expi293 cells and enzymatically biotinylated. Commercial mouse IL-23R-Fc fusion (R&D) was chemically biotinylated via free amines with EZ-Link NHS-LC-Biotin (Thermo Fisher).

### Circular Dichroism

CD spectra were recorded with a J-1500 Circular Dichroism Spectrometer (JASCO). Proteins were assayed at 40 pM in DPBS free of MgCl2 and NaCl. (Life Technologies) with guanidinium hydrochloride from 0 to 6 M, and data were collected at 25° C. For temperature melts, proteins at 40 pM were heated from 25° C. to 95° C. over approximately 1.5 hours.

### Biolayer Interferometry

Qualitative and quantitative assessment of binding affinity was performed using biolayer interferometry (ForteBio Octet™ RED % and associated software for analysis). Enzymatically biotinylated target protein (30 nM) was immobilized on streptavidin-coated sensor tips, then sequentially dipped in wells with: buffer only (baseline), inhibitor in solution (association), and buffer only (dissociation). Kinetic constants were determined from the mathematical fit of a 1:1 binding model.

### Proteolytic Stability Assessment

Simulated intestinal fluid (SIF) was prepared as recommended by Jantratid et al. (termed FaSSIFv2) with the addition of proteases trypsin and chymotrypsin each at 30 μg/mL.¹⁸ This composition is denoted as “1x SIF” in the text. In some instances, designed proteins (pure recombinant protein, or yeast libraries as above) or the comparator V565-38F were so stable that minimal degradation could be detected at the maximum duration (24 hours for SDS PAGE experiments, 90 minutes for cytometry experiments). Therefore, we increased the concentrations of both trypsin and chymotrypsin to increase the rate of digestion; these solutions are denoted as “#x SIF”, where for example “2x SIP” denotes a 2-fold increase in concentration of both trypsin and chymotrypsin (to 60 μg/mL), Simulated gastric fluid was prepared as follows: 600 ug/mL pepsin and 34.2 mM NaCl in water, with HO added to adjust pH to 2.

For qualitative assessment of proteolytic stability, pure recombinant proteins were digested at 37° C. for 24 hours and proteolytic cleavage assessed by SDS PAGE. From concentrated stock solutions, recombinant proteins were added to stock SGF and SIF solutions to a final concentration of 0.1 mg/mL. Timepoints were taken at 0, 5, 15, 30, 60 minutes, 4 and 24 hours; at each timepoint, samples were removed and immediately mixed with load dye and boiled for 5 minutes at 95° C. to quench protease activity. 5 ug protein (based on initial digest concentration of 0.1 mg/mL) per timepoint were run on 16% Tris-tricine polyacrylamide gels.

### IL-23-Mediated Cell Signaling Assay

Commercial IL-23 reporter cells (Promega IL-23 Bioassay) expressing luciferase downstream of IL-23R were used to assess inhibition of IL-23-mediated cell signaling. Cells were plated in the inner wells of 96-well tissue culture treated white plates suitable for reading luminescence. Cells were pre-incubated for 30 minutes with a dilution series of each inhibitor, then treated with the EC80 stimulatory concentration of recombinant human IL-23 cytokine determined in preceding experiments (8 ng/mL; R&D 1290-IL). After 6 hours incubation with human IL-23, luciferase substrate was added and luminescence read. Inhibitor response was plotted as percent maximum it-23 stimulation (without inhibitor) vs. inhibitor concentration, and IC50 values determined by fitting the dose response with nonlinear regression.

### REFERENCES

1. Barrett, J. C. et al. Genome-wide association defines more than 30 distinct susceptibility loci for Crohn's disease. Nat. Genet. 40, 955-962 (2008).
2. Duerr, R. H. et al. A genome-wide association study identifies IL23R as an inflammatory bowel disease gene. Science 314, 1461-1463 (2006).
3. Feagan, B. G. et al. Induction therapy with the selective interleukin-23 inhibitor risankizumab in patients with moderate-to-severe Crohn's disease: a randomised, double-blind, placebo-controlled phase 2 study. Lancet 389, 1699-1709 (2017).
4. Sands, B. E. et al. Efficacy and Safety of MEDI2070, an Antibody Against Interleukin 23, in Patients With Moderate to Severe Crohn's Disease: A Phase 2a Study. Gastroenterology 153, 77-86.e6 (2017).
5. Feagan, B. G. et al. Ustekinumab as Induction and Maintenance Therapy for Crohn's Disease. N Engl. J. Med. 375, 1946-1960 (2016).
6. Houben, R. M. G. J. & Dodd, P. J. The Global Burden of Latent Tuberculosis Infection: A Re-estimation Using Mathematical Modelling. PLoS Med. 13, e1002152 (2016).
7. Zampino, R. et al. Hepatitis B virus burden in developing countries. World J. Gastroenterol. 21, 11941-11953 (2015).
8. Baert, F. et al. Influence of immunogenicity on the long-term efficacy of infliximab in Crohn's disease. N Engl. J. Med. 348, 601-608 (2003).
9. Office of the Commissioner. Xeljanz, Xeljanz XR (tofacitinib): Drug Safety Communication. U.S. Food and Drug Administration https://www.fda.gov/safety/medical-product-safety-information/xeljanz-xeljanz-xr-tofacitinib-drug-safety-communication-due-increased-risk-blood-clots-and-death (2019).
10. Bourne, G. T. et al. Oral peptide inhibitors of interleukin-23 receptor and their use to treat inflammatory bowel diseases. US Patent (2017).
11. Fleishman, S. J. et al. Computational design of proteins targeting the conserved stem region of influenza hemagglutinin. Science 332, 816-821 (2011).
12. Strauch, E. M. et al. Computational design of trimeric influenza-neutralizing proteins targeting the hemagglutinin receptor binding site. Nat. Biotechnol. 35, 667-671 (2017).
13. Procko, E. et al. A computationally designed inhibitor of an Epstein-Barr viral Bcl-2 protein induces apoptosis in infected cells. Cell 157, 1644-1656 (2014).
14. Chevalier, A. et al. Massively parallel de novo protein design for targeted therapeutics. Nature 550, 74-79 (2017).
15. Rocklin, G. J. et al. Global analysis of protein folding using massively parallel design, synthesis, and testing. Science 357, 168-175 (2017).
16. Bloch, Y. et al. Structural Activation of Pro-inflammatory Human Cytokine IL-23 by Cognate IL-23 Receptor Enables Recruitment of the Shared Receptor IL-12Rβ1. Immunity 48, 45-58.e6 (2018).
17. Dou, J. et al. De novo design of a fluorescence-activating β-barrel. Nature 561, 485-491 (2018).
18. Jantratid, E., Janssen, N., Reppas, C. & Dressman, J. B. Dissolution media simulating conditions in the proximal human gastrointestinal tract: an update. Pharm. Res. 25, 1663-1676 (2008).
19. Degen L. P., P. S. F. Variability of gastrointestinal transit in healthy women and men. Gut 39, 299-305 (1996).
20. Crowe, J. S. et al. Preclinical Development of a Novel, Orally-Administered Anti-Tumour Necrosis Factor Domain Antibody for the Treatment of Inflammatory Bowel Disease. Sci. Rep. 8, 4941 (2018).
21. Nurbhai, S. et al. Oral Anti-Tumour Necrosis Factor Domain Antibody V565 Provides High Intestinal Concentrations, and Reduces Markers of Inflammation in Ulcerative Colitis Patients. Sci. Rep. 9, 14042 (2019).
22. A Study Evaluating Participants With Moderately to Severely Active Crohn's Disease—Full Text View—ClinicalTrials.gov. https://clinicaltrials.gov/ct2/show/NCT04102111.
23. A Six Week Efficacy, Safety and Tolerability Study of V565 in Crohn's Disease—Full Text View—ClinicalTrials.gov. https://clinicaltrials.gov/ct2/show/NCT02976129?term=V565&draw=2&rank=2.
24. Berger, S. et al. Computationally designed high specificity inhibitors delineate the roles of BCL2 family proteins in cancer. Elife 5, (2016).
25. Procko, E. et al. Computational design of a protein-based enzyme inhibitor. J. Mol. Biol. 425, 3563-3575 (2013).
26. Chao, G. et al. Isolating and engineering human antibodies using yeast surface display. Nat. Protoc. 1, 755-768 (2006).
27. Benatuil, L., Perez, J. M., Belk, J. & Hsieh, C. M. An improved yeast transformation method for the generation of very large human antibody libraries. Protein Eng. Des. Sel. 23, 155-159 (2010).

1. A human IL-23R (hIL-23R) binding polypeptide, comprising a polypeptide of the general formula X1-X2-X3-X4-X5, wherein X1, X2, X3, and X4 are optional, wherein X5 comprises a polypeptide domain of between 12-20 amino acids in length, and wherein X5 comprises the amino acid sequence of residues 40-47 in SEQ ID NO:1 or 2.

2. The hIL-23R binding polypeptide of claim 1, wherein X5 comprises the amino acid sequence of residues 40-47 in the amino acid sequence selected from the group consisting SEQ ID NO: 3-6.

3. The hIL-23R binding polypeptide of claim 1, wherein
(a) X3 is present, wherein X3 comprises a polypeptide domain between 12-20 amino acids in length, and wherein X4 is either absent, or comprises an amino acid linker; optionally wherein X3 comprises a polypeptide having the amino acid sequence of residues 22-33 in the amino acid sequence selected from the group consisting of SEQ ID NOS:1-6; optionally wherein X3 comprises the amino acid sequence of residues 21-35 in the amino acid sequence selected from the group consisting of SEQ ID NOS:1-6;
(b) wherein X1 is present and comprises a polypeptide domain of between 12-20 amino acids in length; optionally wherein X1 comprises the amino acid sequence or residues 1-16 in the amino acid sequence selected from the group consisting of SEQ ID NOS:1-6; optionally wherein X2 is present, and wherein X2 comprises an amino acid linker; and/or
(c) wherein X5 comprises the amino acid sequence of residues 39-54 in the amino acid sequence selected from the group consisting of SEQ ID NOS:1-6.

4-12. (canceled)

13. The hIL-23R binding polypeptide of claim 1, wherein X3 is present, and wherein:
(I)(a) X5 comprises the amino acid sequence of residues 40-47 in the amino acid sequence selected from the group consisting SEQ ID NO: 5-6; and
(b) X3 comprises the amino acid sequence of residues 22-33 in the amino acid sequence selected from the group consisting SEQ ID NO: 5-6; or
(II)(a) X5 comprises the amino acid sequence of residues 39-54 in the amino acid sequence selected from the group consisting SEQ ID NO: 5-6; and
(b) X3 comprises the amino acid sequence of residues 21-35 in the amino acid sequence selected from the group consisting SEQ ID NO: 5-6.

14. (canceled)

15. The hIL-23R binding polypeptide of claim 13, wherein X1 is present, and wherein X1 comprises the amino acid sequence of residues 1-16 in the amino acid sequence selected from the group consisting of SEQ ID NOS:5-6.

16. (canceled)

17. The hIL-23R binding polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:10-74, optionally wherein 1, 2, 3, 4, 5, 6, 7, 8, 9, 10, or more of the N-terminal amino acids may be deleted from the polypeptide, and thus may be deleted from the reference polypeptide when considering percent identity.

18-21. (canceled)

22. The hIL-23R binding polypeptide of claim 1, further comprising one or more additional functional domains added at the N-terminus and/or the C-terminus of the polypeptide, optionally wherein the additional functional domain comprises a targeting domain.

23. (canceled)

24. The hIL-23R binding polypeptide of claim 1, wherein each of X1, X2, X3, X4, and X5 are present, and wherein
X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X1 domain present in any of SEQ ID NOS: 10-74;
X2 comprises an amino acid sequence at least 50%, 75%, or 100% identical to the amino acid sequence of an X2 domain present in any of SEQ ID NOS: 10-74,
X3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X3 domain present in any of SEQ ID NOS: 10-74,
X4 comprises an amino acid sequence at least 33%, 66%, or 100% identical to the amino acid sequence of an X4 domain present in any of SEQ ID NOS: 10-74, and
X5 comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of an X5 domain present in any of SEQ ID NOS: 10-74.

25. (canceled)

26. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to:
the amino acid sequence of an X5 domain present in a polypeptide selected from the group consisting of SEQ ID NO:10-74, or selected from SEQ ID NO: 69 and 74;
the amino acid sequence of an X4-X5 domain combination present in a polypeptide selected from the group consisting of SEQ ID NO:10-74, or selected from SEQ ID NO: 69 and 74;
the amino acid sequence of an X3-X4-X5 domain combination present in a polypeptide selected from the group consisting of SEQ ID NO:10-74, or selected from SEQ ID NO: 69 and 74; or
the amino acid sequence of an X2-X3-X4-X5 domain combination present in a polypeptide selected from the group consisting of SEQ ID NO:10-74, or selected from SEQ ID NO: 69 and 74.

27. (canceled)

28. The hIL-23R binding polypeptide of claim 1, wherein the polypeptide is an hIL-23R antagonist.

29. An hIL-23R binding polypeptide, comprising a polypeptide of the general formula X1-X2-X3-X4-X5, wherein X2, X3, X4, and X5 are optional, wherein X1 comprises a polypeptide domain of between 12-20 amino acids in length, and wherein X1 comprises the amino acid sequence of residues 1-10 in SEQ ID NO:101 or 102, or wherein X1 comprises the amino acid sequence of residues 1-10 in the amino acid sequence selected from the group consisting of SEQ ID NOS: 103-108.

30-60. (canceled)

61. An hIL-23R binding polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:84-87 and 181-228, wherein 1, 2, 3, or more of the N-terminal and/or C-terminal amino acids may be deleted from the polypeptide, and thus may be deleted from the reference polypeptide when considering percent identity, optionally comprising a disulfide bond between two cysteine residues in the polypeptide, optionally wherein the polypeptide is a hIL-23R antagonist.

62-65. (canceled)

66. A conditionally maximally active hIL-23R binding protein, comprising a first polypeptide component and a second polypeptide component, wherein the first polypeptide component and the second polypeptide component are not present in a fusion protein, wherein
(a) in total the first polypeptide component and the second polypeptide component comprise domains X3 and X5 of claim 3;
(b) the X3 domain is present in the first polypeptide component and the X5 domain is present in the second polypeptide component;
the first polypeptide component and the second polypeptide component are not maximally active hIL-23R binding protein individually, and wherein the first polypeptide component and the second polypeptide interact to form a maximally active hIL-23R binding protein.

67-76. (canceled)

77. A conditionally maximally active hIL-23R binding protein, comprising a first polypeptide component and a second polypeptide component, wherein the first polypeptide component and the second polypeptide component are not present in a fusion protein, wherein
(a) in total the first polypeptide component and the second polypeptide component comprise domains X1 and X3 of claim 31;
(b) the X1 domain is present in the first polypeptide component and the X3 domain is present in the second polypeptide component;
the first polypeptide component and the second polypeptide component are not maximally active hIL-23R binding protein individually, and wherein the first polypeptide component and the second polypeptide non-covalently interact to form a maximally active hIL-23R binding protein.

78-112. (canceled)

113. A multimer comprising two or more copies of the hIL-23R binding polypeptide of claim 1.

114. A nucleic acid encoding the polypeptide of claim 1.

115. An expression vector comprising the nucleic acid of claim 114 operatively linked to a suitable control element.

116. A cell comprising the expression vector of claim 115.

117. A pharmaceutical composition comprising:
(a) the polypeptide of claim 1; and
(b) a pharmaceutically acceptable carrier.

118. A method for treating a disorder selected from the group consisting of inflammatory bowel disease (IBD) (including but not limited to includes Crohn's disease and ulcerative colitis), psoriasis, atopic dermatitis, rheumatoid arthritis, psoriatic arthritis, osteoarthritis, axial and peripheral spondyloarthritis, ankylosing spondylitis, enthesitis, and tendonitis, comprising administering to a subject in need thereof an amount effective to treat the disorder of the polypeptide of claim 1.
